(19) United States
(12) Patent Application Publication
(10) Pub. No.: US 2019/0330706 A1
(43) Pub. Date: Oct. 31, 2019

(54) NUCLEIC ACID AMPLIFICATION ASSAYS FOR DETECTION OF PATHOGENS

(71) Applicants: THE BROAD INSTITUTE, INC., Cambridge, MA (US); MASSACHUSETTS INSTITUTE OF TECHNOLOGY, Cambridge, MA (US); PRESIDENT AND FELLOWS OF HARVARD COLLEGE, Cambridge, MA (US)

(72) Inventors: Pardis SABETI, Cambridge, MA (US); Hayden METSKY, Cambridge, MA (US); Mary Lynn BANIECKI, Cambridge, MA (US)

(21) Appl. No.: 16/328,642

(22) PCT Filed: Aug. 25, 2017
(86) PCT No.: PCT/US17/48749

§ 371 Date: Feb. 26, 2019

Related U.S. Application Data
(60) Provisional application No. 62/380,352, filed on Aug. 26, 2016.
(60) Provisional application No. 62/459,578, filed on Feb. 15, 2017.
(60) Provisional application No. 62/507,619, filed on May 17, 2017.

(51) Int. Cl. C12Q 1/70; G16B 30/00

(52) U.S. Cl. CPC C12Q 1/701; C12Q 2600/156; G16B 30/00

(57) ABSTRACT

The present invention relates to a method for generating primers and/or probes for use in analyzing a sample which may comprise a pathogen target sequence comprising providing a set of input genomic sequence to one or more target pathogens, generating a set of target sequences from the set of input genomic sequences, identifying one or more highly conserved target sequences, and generating one or more primers, one or more probes, or a primer pair and probe combination based on the one or more conserved target sequences.

### CROSS REFERENCE TO RELATED APPLICATIONS

This application claims the benefit of U.S. Provisional Application No. 62/380,352, filed Aug. 26, 2016, U.S. Provisional Application No. 62/459,578, filed Feb. 15, 2017, and U.S. Provisional Application No. 62/507,619, filed May 17, 2017. The entire contents of the above-identified applications are hereby fully incorporated herein by reference.

### STATEMENT REGARDING FEDERALLY SPONSORED RESEARCH

This invention was made, in whole or in part, with government support under grant number U19AI110818 granted by the National Institute of Allergy and Infectious Diseases, National Institutes of Health, Department of Health and Human Services. The government has certain rights in the invention.

### FIELD OF THE INVENTION

The present invention provides a combination of genomic and computational technologies to provide rapid, portable sample analysis for identifying a target sequence.

### BACKGROUND OF THE INVENTION

Infectious diseases cause tremendous morbidity and mortality in tropical developing countries, and the need for a holistic approach to their detection and diagnosis is increasingly clear. The full range and prevalence of pathogens in such settings is not well understood, and the capacity to detect new or infrequent threats, like Ebola, is often lacking. The ability to diagnose a broad spectrum of pathogens is vital, since infection with multiple pathogens and resulting misdiagnoses are common.

First, there is a need in patient care for more comprehensive diagnostic tests. Many pathogens produce non-specific symptoms like fever, headache, and nausea, making them difficult to distinguish clinically. For example, 30%-90% of hospitalized patients with acute fever in tropical Africa are diagnosed with malaria and treated accordingly, while only 7%-45% of them actually have laboratory-confirmed malaria. Better tests for individual diseases will be useful, but will not fully solve the problem: e.g., many patients with detectable malaria are actually sick because of other infections. Such misdiagnoses can be fatal, as in a 1989 outbreak of Lassa fever in two Nigerian hospitals, where 22 people died. Thus, Applicants have developed a low-cost PCR-based panel for a range of infectious diseases as a routine diagnostic procedure for febrile patients.

Second, there is a need to better understand the array of existing pathogens and to detect emerging threats. Lassa virus, once thought to be a novel cause of sporadic disease outbreaks, has turned out to be endemic in much of West Africa, and there is even evidence that Ebola circulates undetected more widely than is supposed. Any samples that fail Applicants' diagnostic panel, therefore, are sent for deep metagenomic sequencing to detect other pathogens. A random selection of other samples is treated the same way, to provide a broad picture of the range of pathogens in the region, which in turn will enable early detection of new or increasing pathogens.

Technological advances in sequencing and analyzing the genomes of a wide variety of microbes, including the costs of implementing genomic approaches at scale, make it possible to address these needs. However, to fulfill that promise, the tools must be delivered to researchers and clinicians on the ground. Empowering local health care clinics and their communities, in turn, will help motivate patients to seek care at the clinic. In addition to saving lives, this enables us to continually monitor patients with unexplained fever, capturing diseases that previously went undiagnosed or misdiagnosed. After local diagnosis, samples can then be sent to advanced laboratories in the US—and hopefully soon Africa too—for in-depth analysis using high-throughput metagenomic sequencing. Discoveries of new pathogens can then be converted into affordable, field-deployable diagnostics to inform health care workers and the populations they serve, reducing the burden of disease, and improving local capacity to detect and treat at the earliest possible stages. Robust data systems are needed to connect sample collections, the process of pathogen identification, and candidates for developing diagnostics and treatments. By comprehensively identifying pathogens circulating in the population this new infrastructure serves as an early warning for emerging and persistent diseases. With their own diagnostic capacity for a wide range of infectious agents, sites throughout Africa are able to support their communities and help to detect, monitor and characterize emerging diseases before they become global threats.

### SUMMARY OF THE INVENTION

Embodiments disclosed herein are directed to methods of identifying highly conserved regions among pathogen variants and/or pathogen species and use of primers and probes directed to such regions for the development and use of nucleic acid-based detection assays for detection of pathogens.

In one aspect, the invention provides a method for developing probes and primers to pathogens, comprising: providing a set of input genomic sequences to one or more target pathogens; generating a set of target sequences from the set of input genomic sequences; applying a set cover solving process to the set of target sequences to identify one or more target amplification sequences, wherein the one or more target amplification sequences are highly conserved target sequences shared between the set of input genomic sequences of the target pathogen; and generating one or more primers, one or more probes, or a primer pair and probe combination based on the one or more target amplification sequences. In one embodiment, the set of input genomic sequences represent genomic sequences from two or more variants of the one or more target pathogens. In another embodiment, the set of input genomic sequences are obtained from a metagenomic sample. In another embodiment, the metagenomic sample is obtained from one or more vector species of the one or more target pathogens. In another embodiment, the one or more vector species are one or more species of mosquito. In another embodiment, the one or more target pathogens is one or more viral pathogens. In another embodiment, the viral pathogen is Zika, Chikungunya, or Dengue. In another embodiment, the one or more viral pathogens is Zika, Chikungunya. In another embodiment, the one or more target pathogens is a parasitic pathogen. In another embodiment, the target sequences are fragmented to a size that is approximately equal to a size of an amplicon for detection using a nucleic acid amplification assay, such as a target sequence size of 100 to 500 base pairs. In another embodiment, each nucleotide of the set of input genomic sequences is considered an element of universe of the set cover solving process and wherein each element is considered covered if the target sequence aligns to some portion of a genomic reference sequence.

In another aspect, the invention provides a method for detecting one or more pathogens comprising: contacting a sample with one or more primers and/or probes generated using a method as described herein; detecting amplification of one or more pathogen target sequences using a nucleic acid amplification method and the one or more primers and/or probes, wherein detection of the target sequence indicates a presence of the one or more pathogens in the sample. In one embodiment, the nucleic acid amplification method is quantitative PCR and the one or more primers and/or probes comprise a forward and reverse primers and a probe modified with a detectable label. In one embodiment, the forward primer comprises one of SEQ ID NOs: 3, 7, 11, 15, 19, 23, 27, 31, 35, 39, or 43, the reverse primer comprises one of SEQ ID NOs: 4, 8, 12, 16, 20, 24, 28, 32, 36, 40, or 44, and the probe comprises one of SEQ ID NOs: 5, 9, 13, 17, 21, 25, 29, 33, 37, 41, 45, or 47. In another embodiment, the one or more primers and/or probes are configured to detect one or more non-synonymous single nucleotide polymorphisms (SNPs) listed in Tables 3 or 7.

In another aspect, the invention provides a method for detecting Zika, Chikungunya, Dengue, or a combination thereof in samples, comprising contacting a sample with a forward and reverse primer and a probe with a detectable label, wherein the forward primer comprises one or more of SEQ ID NOs: 3, 7, 11, 15, 19, 23, 27, 31, 35, 39, or 43 the reverse primer comprises one of more of SEQ ID NOs: 4, 8, 12, 16, 20, 24, 28, 32, 36, 40, or 44 and the probe comprises one or more of 5, 9, 13, 17, 21, 25, 29, 33, 37, 41, 45, or 47.; and detecting amplification of one or more target sequences through a quantitative PCR assay using the forward and reverse primers and the probe, wherein detection of the one or more target sequences indicates the presence of Zika, Chikungunya, or both. In another example embodiment, a method for detecting Zika and/or Chikungunya in samples comprises contacting a sample with a forward and reverse primer and a probe with a detectable label, wherein the forward primer, reverse primer, and probe are each configured to hybridize to at least a portion of one or more of the target sequences of SEQ ID NOs: 6, 10, 14, 18, 22, 26, 30, 34, 38, 42, or 46; and detecting amplification of the one or more target sequences through a quantitative PCR assay using the forward and reverse primers and the probe, wherein detection of the one or more target sequences indicates the presence of Zika, Chikungunya, Dengue or a combination thereof in the sample.

In another aspect, the invention provides a method for detecting Dengue

In another aspect, the invention provides a kit comprising the primers and/or probes as described herein.

These and other aspects, objects, features, and advantages of the example embodiments will become apparent to those having ordinary skill in the art upon consideration of the following detailed description of the illustrated embodiments.

### BRIEF DESCRIPTION OF THE DRAWINGS

FIG. 1—Shows the background of Zika virus.

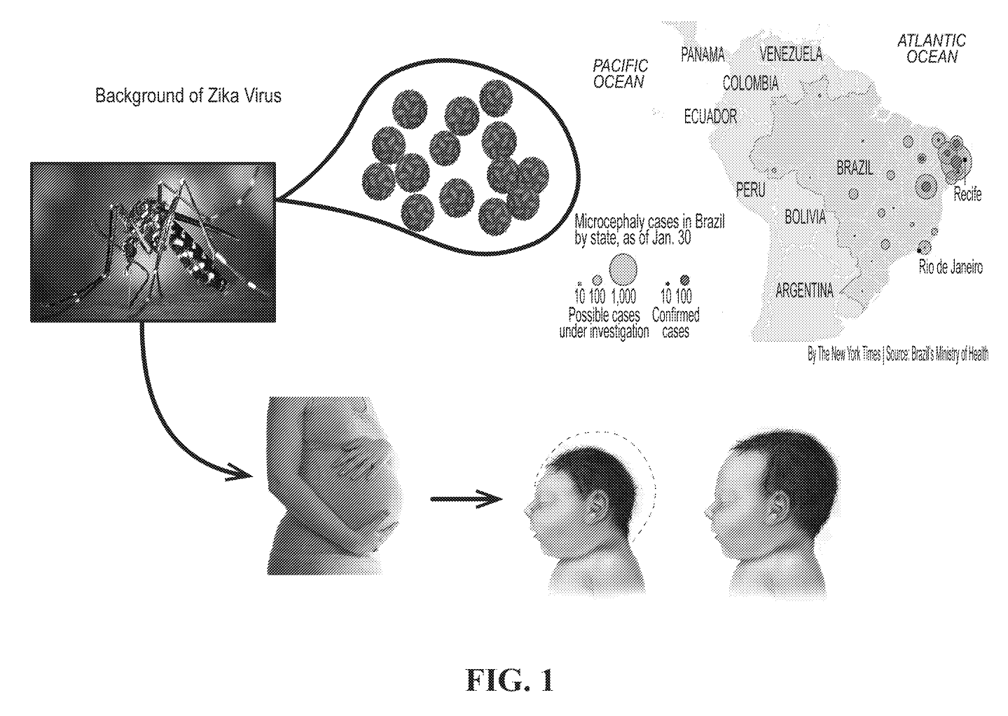

FIG. 2—Shows the global health perspective of Zika virus.

FIG. 3—Shows an overview of the diagnostics of Zika virus.

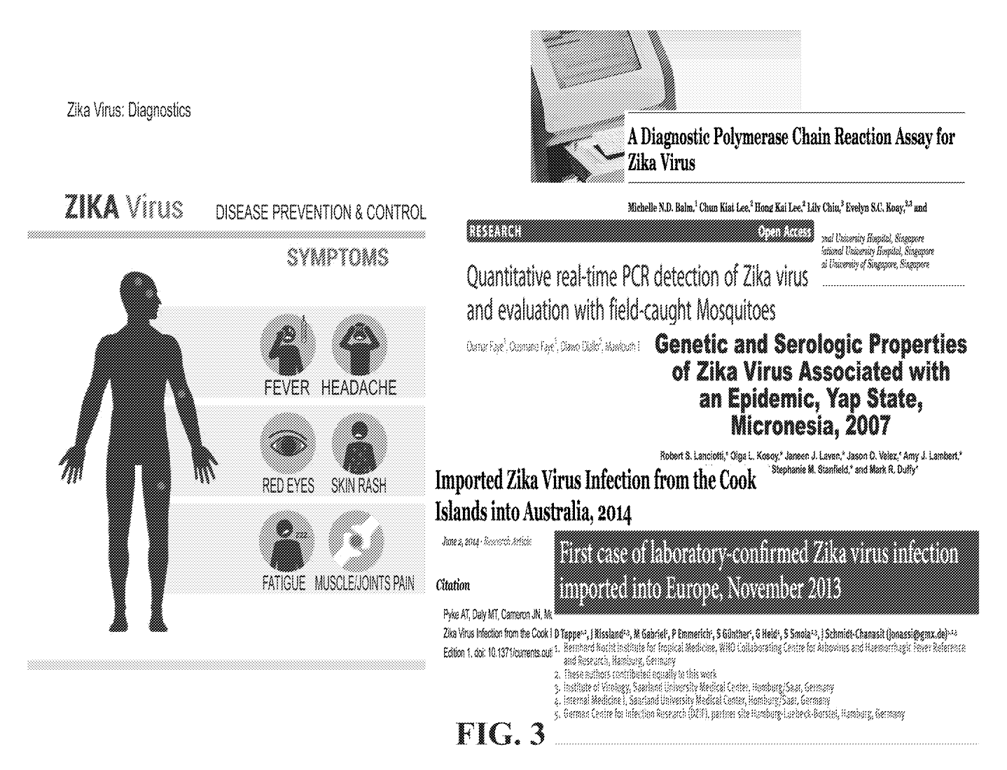

FIG. 4—Shows a diagram of the Zika virus genome.

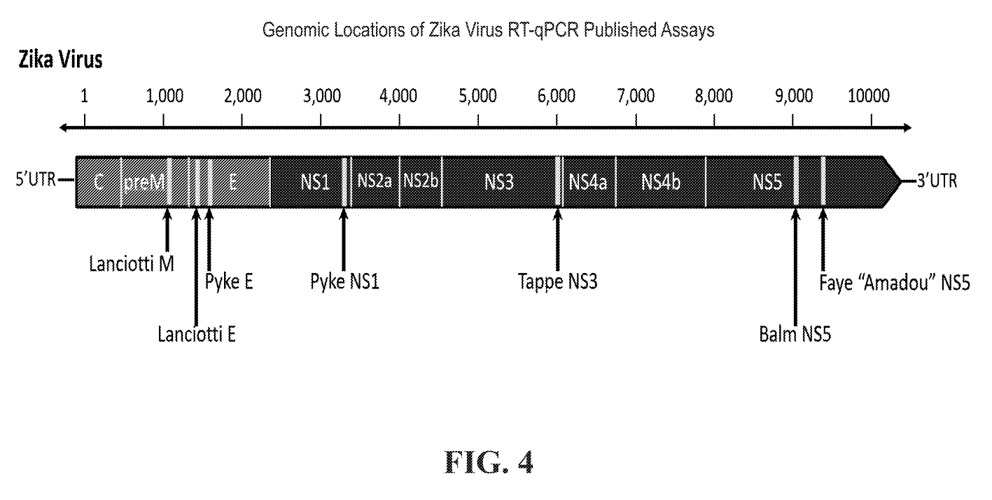

FIG. 5—Shows a plot of the percent genomic identity of all global Zika virus strains.

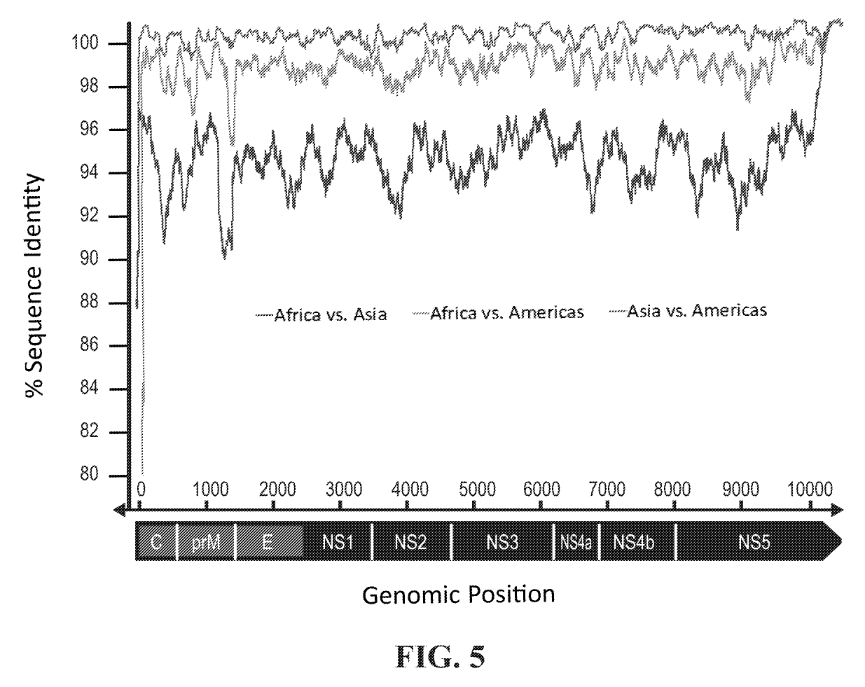

FIG. 6—Shows Zika RT-qPCR assays and nucleotide mismatches across Zika strains.

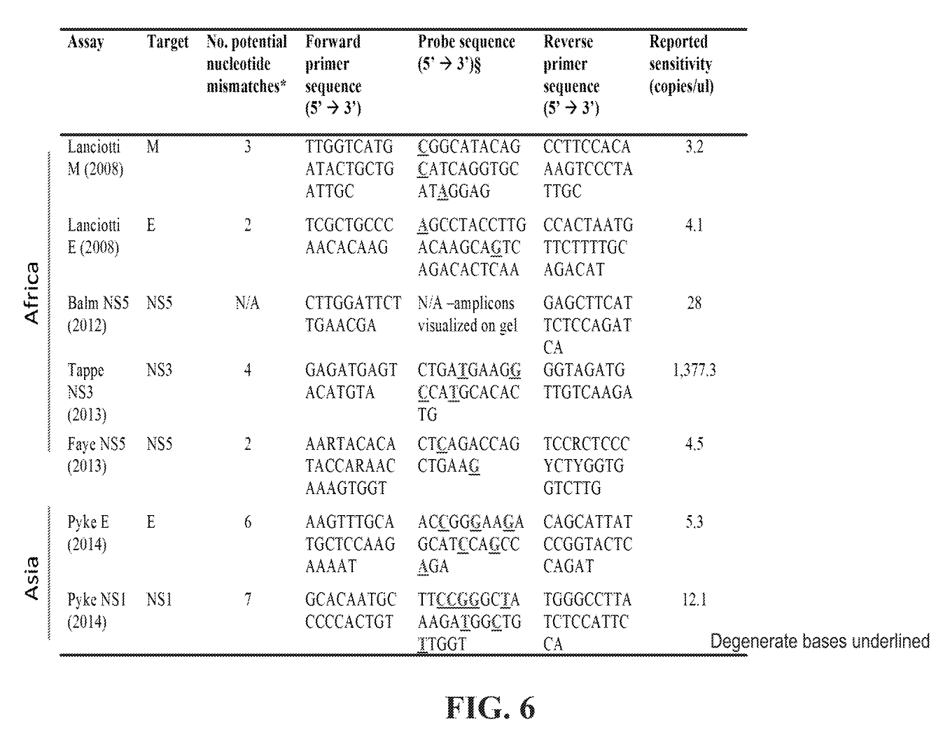

FIG. 7—Shows performance data for Zika RT-qPCR assays.

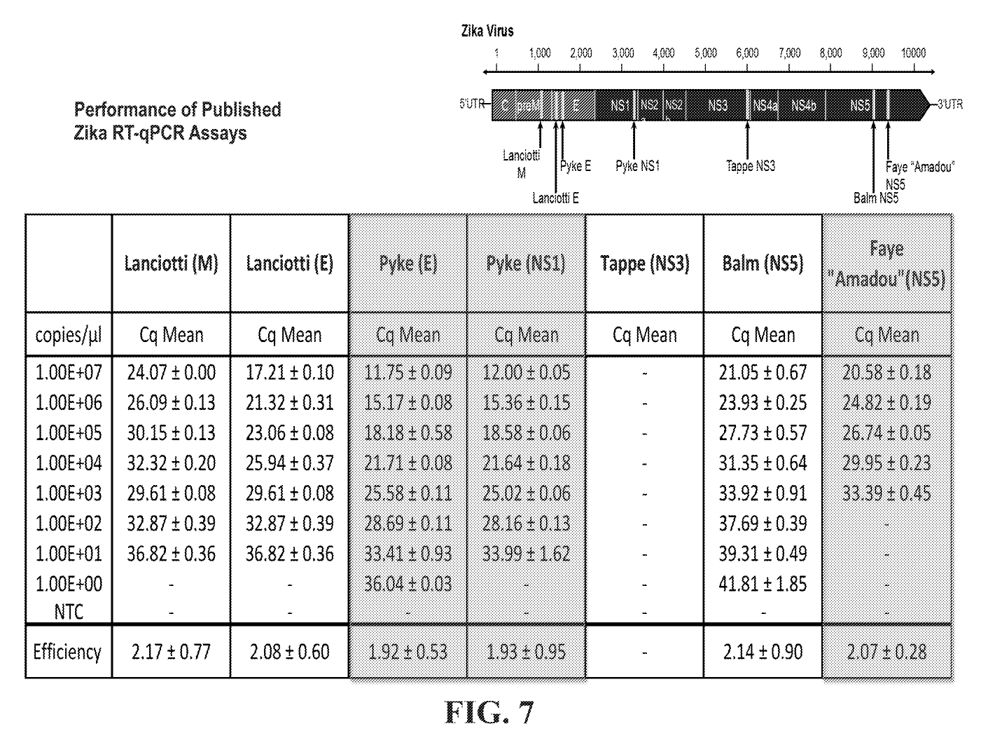

FIG. 8—Shows standard curves for three Zika assays, FAYE, Pyke E, and NS1.

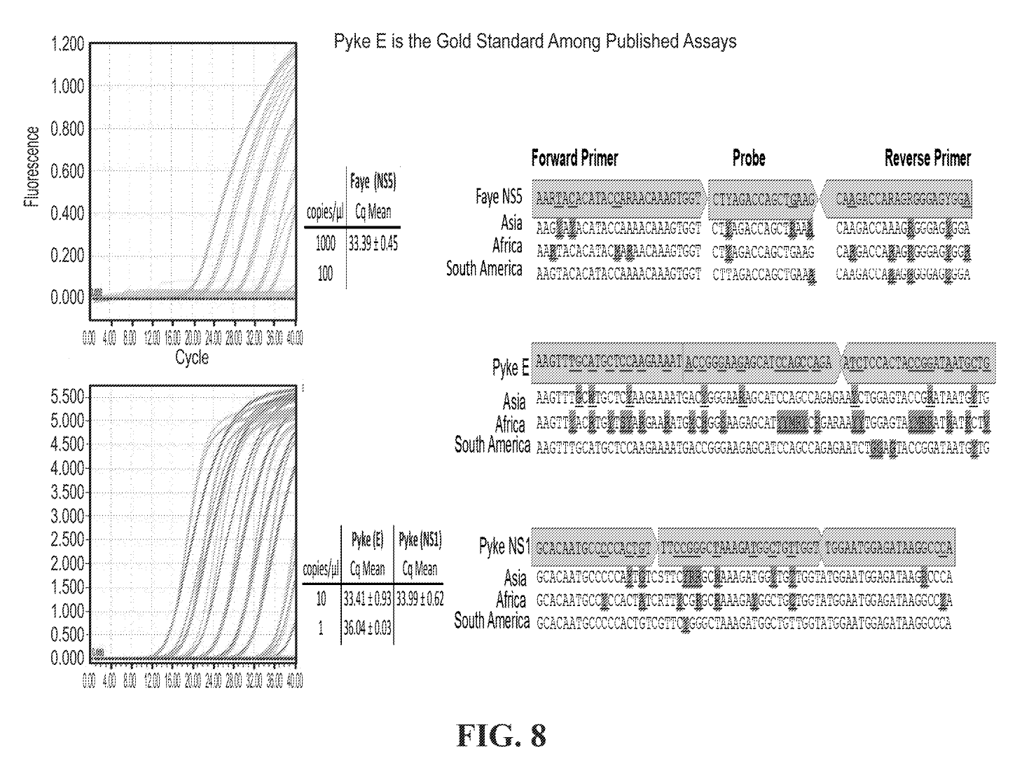

FIG. 9—Shows a workflow for RT-qPCR diagnostic development.

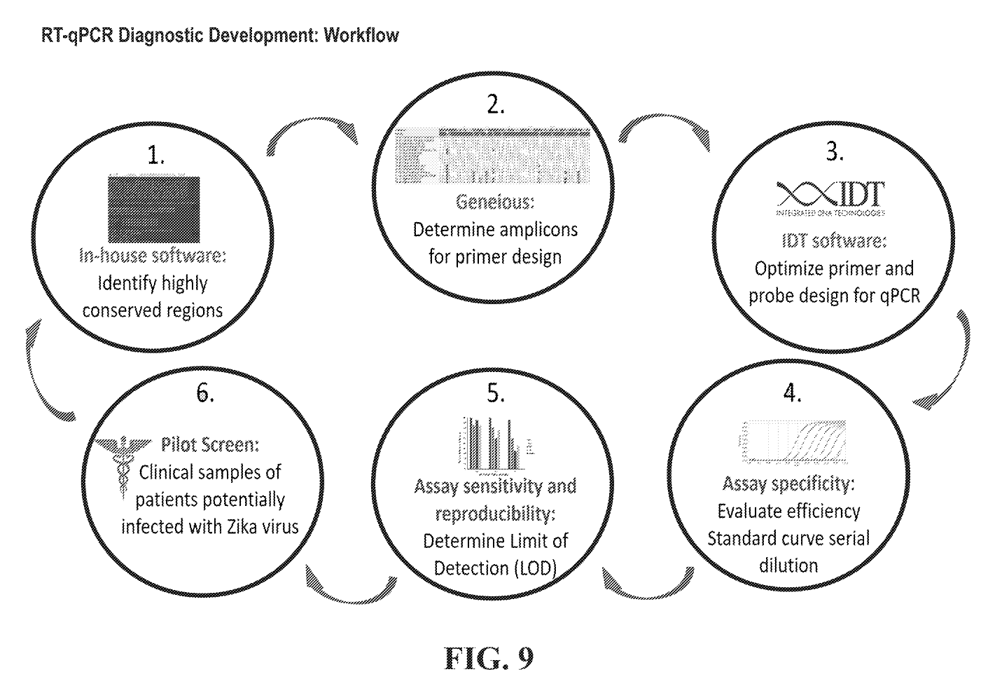

FIG. 10—Shows design for new Zika RT-qPCR assays.

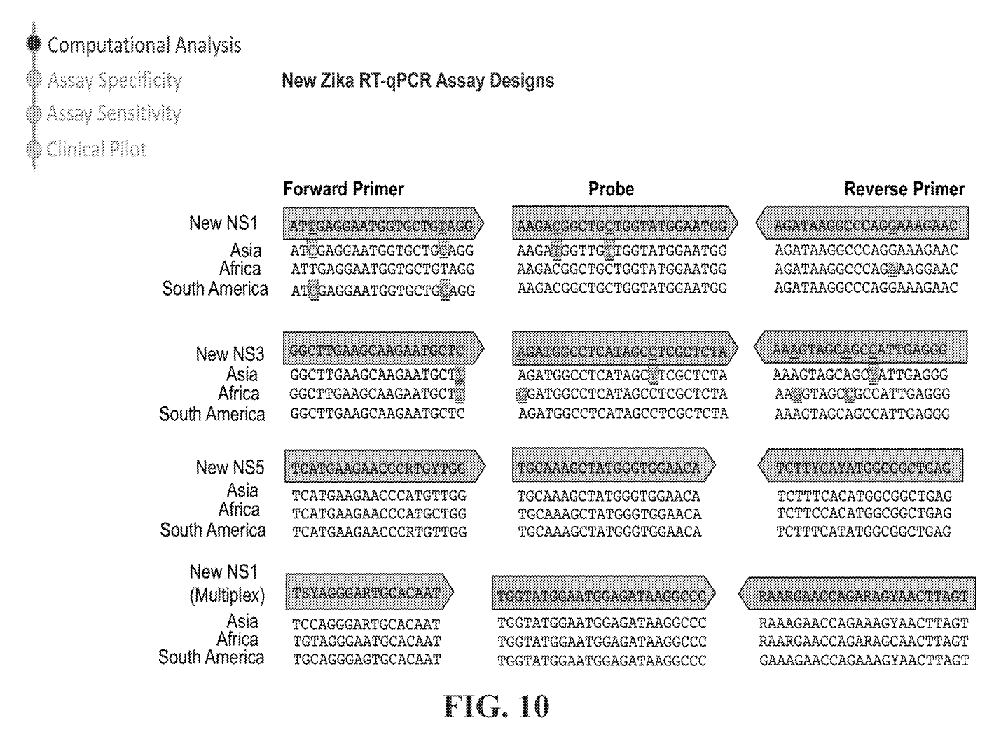

FIG. 11—Shows results from newly designed assays against NS1, NS3, NS5 regions of Zika virus.

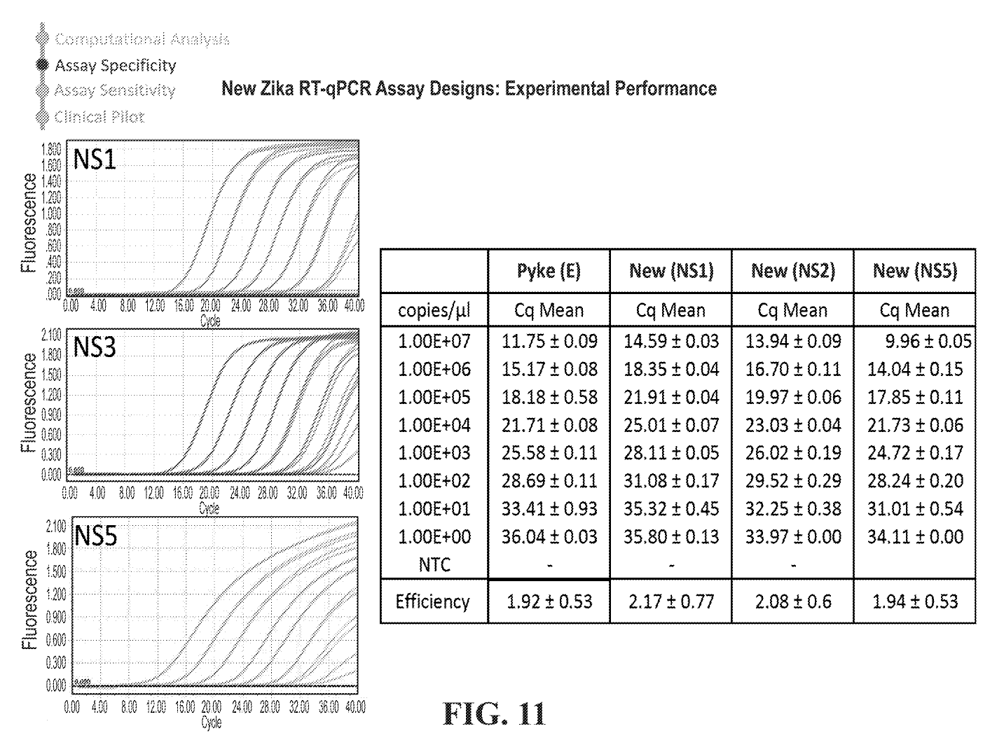

FIG. 12—Shows the limit of detection of Zika RT-qPCR assays. The NS5 assay was found to be the most robust.

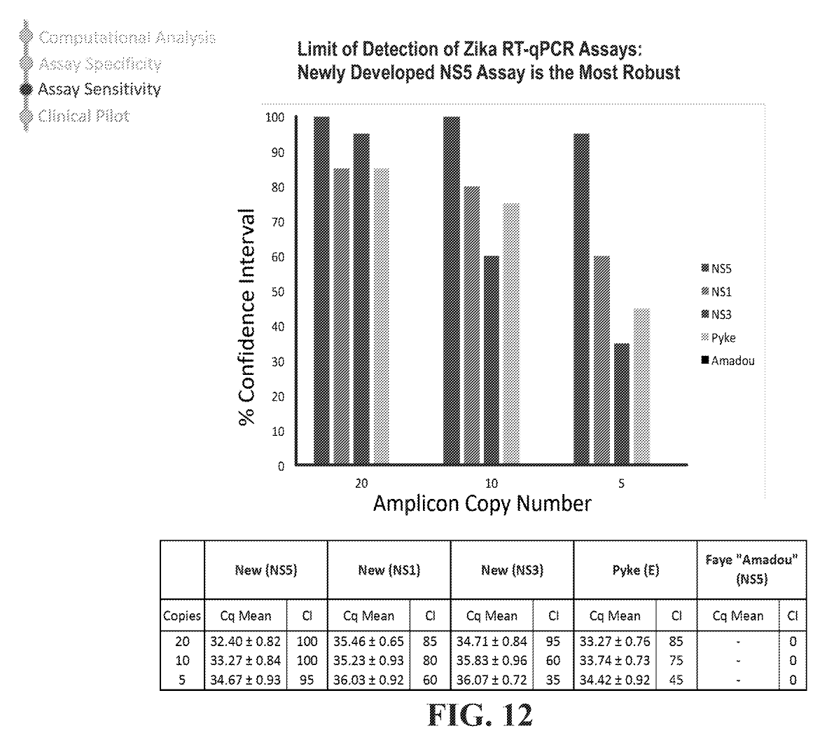

FIG. 13—Shows results of Zika NS5 probe-based diagnostic assay.

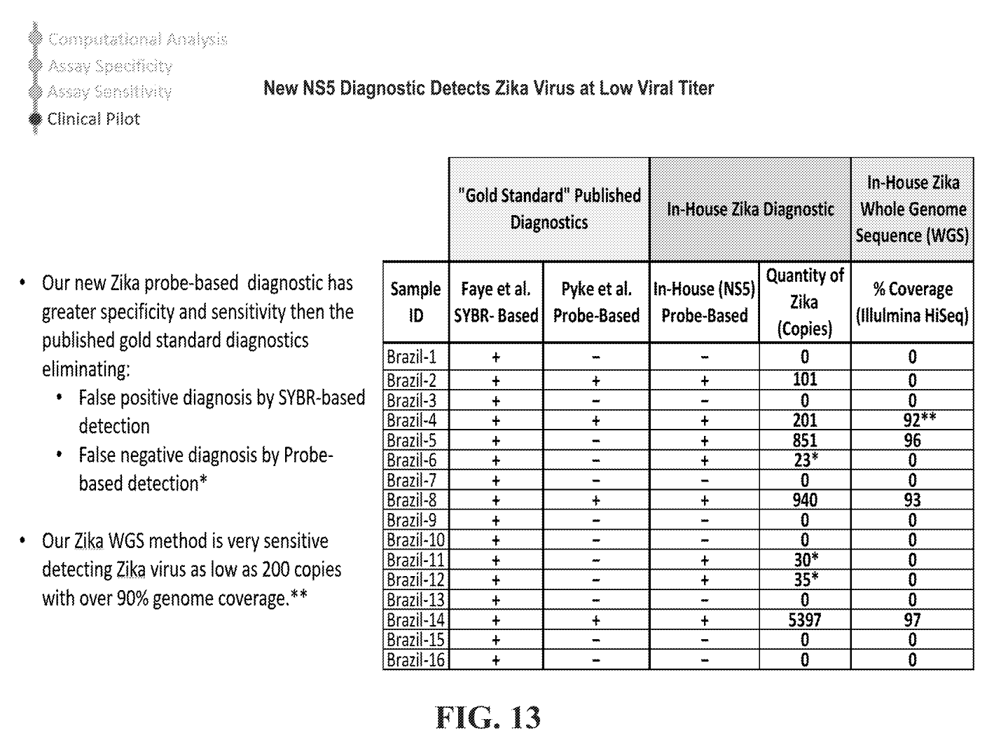

FIG. 14—Shows results of Zika NS5 probe-based diagnostic assay with concentration values.

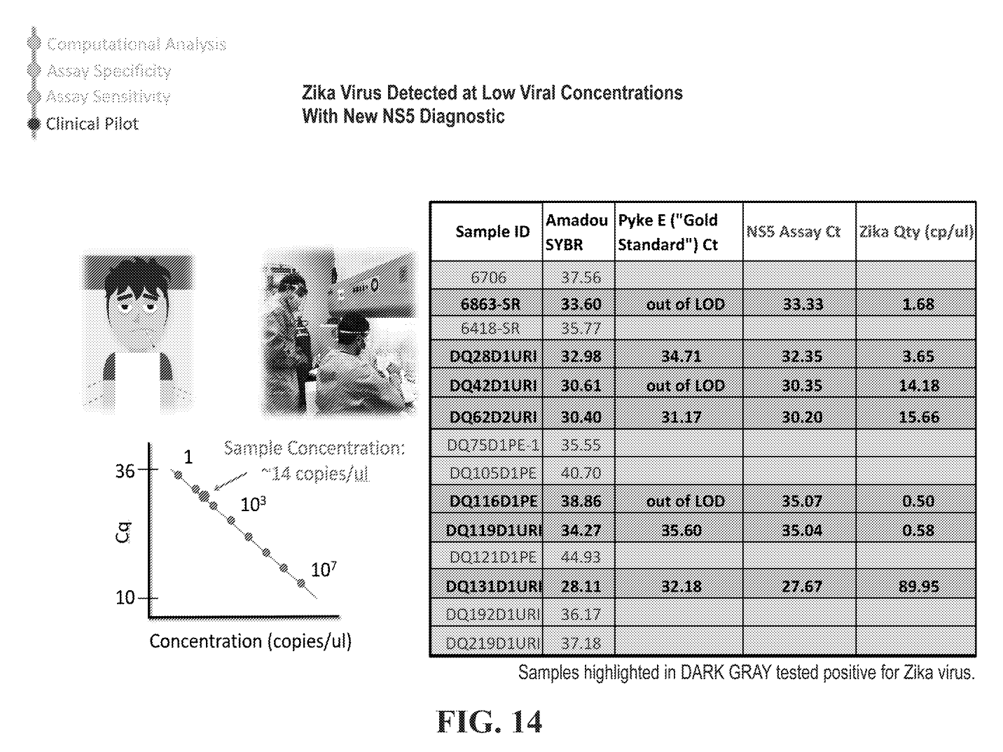

FIG. 15—Shows primers and probes for detection of Zika virus.

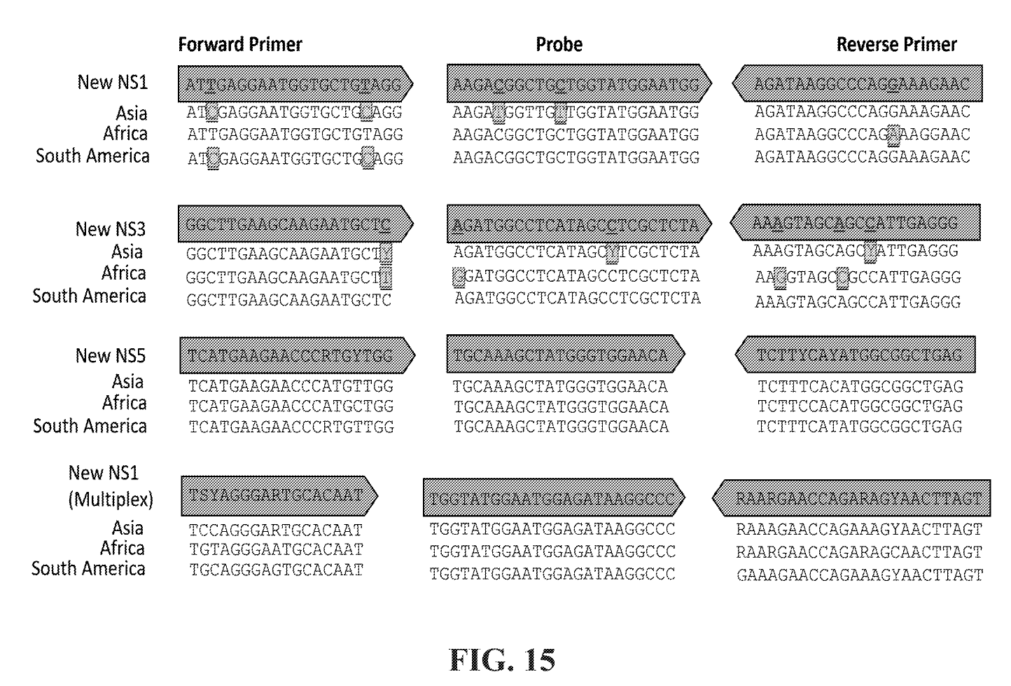

FIG. 16—Shows sequencing data generated directly from clinical samples. 200 clinical and mosquito pool samples were sequenced using amplicon and/or hybrid capture sequencing methods, generating 100 ZIKV genomes. (a) For each country, the number of genomes generated by each sequencing method; each genome counted is from a sample that has at least one “positive” assembly, i.e. a replicate passes thresholds in (b). The “Other” category includes all samples from countries that did not produce a positive assembly. In the final column, genomes are counted only once if both methods produced a positive assembly. (b) Thresholds used to select samples for downstream analysis. Each point is a replicate. Red and blue shading: regions of accepted amplicon sequencing and hybrid capture genome assemblies, respectively; purple: positive assemblies by either method. Not shown: hybrid capture positive controls with depth >10,000×. (c) Amplicon sequencing coverage by sample across the ZIKV genome. Red indicates sequencing depth ≥500×, and the heat map (bottom) sums coverage across all samples; white horizontal lines indicate amplicon locations. (d) Relative sequencing depth across hybrid capture genomes. (e) Within-sample variant frequencies across methods. Each point is a particular variant in an individual sample and points are plotted on a log-log scale. Green points represent “verified” variants detected by hybrid capture sequencing that pass strand bias and single-library frequency filters. (f) Within-sample variant frequencies across replicate libraries per method. Red points are variants identified using amplicon sequencing; blue points are variants identified using hybrid capture. Light colored points do not pass a strand bias filter; dark points do. In (e-f), frequencies <0.5% are shown at 0%.

FIG. 17—Shows the relationship between metadata and sequencing outcome. The significance of the site where a sample was collected, patient gender, patient age, sample type, and days between symptom onset and sample collection (“collection interval”) were tested as predictors of sequencing outcome. (a) To predict whether a sample is positive by sequencing, a full model was constructed with all predictors and likelihood ratio tests were performed on each predictor by subtracting it from the full model. Sample site and patient gender improved the model. (b) For each of six sample sites, division was done by gender and a point was shown for each sample at its response value in the model. Shaded region below dotted line shows sequencing-negative values used in this model; region above is positive. The discrepancy in positivity between females and males is driven largely by Sample sites 2, 5, and 6. (c) Using only the observed positive samples, percent genome identified was predicted. Likelihood ratio tests were performed, as in (a), and it was found that collection interval improved the model. (d) Sequencing outcome for each sample by collection interval, separated by sample site. Samples collected 7+ days after symptom onset produced, on average, the fewest unambiguous bases, though these observations were based on a limited number of data points. While the sample site variable accounted for differences in the composition of cohorts, the effects of gender and collection interval might be due to confounders in composition that span multiple cohorts.

FIG. 18—Shows Zika virus spread throughout the Americas. (a) Samples were collected in each of the colored countries or territories. Darker regions indicate the specific state, department, or province of sample origin, if known. (b) Maximum clade credibility tree generated using BEAST shows Zika virus introductions from Brazil and into various South and Central American countries and regions. Tips with bolded branches and labels correspond to sequences generated in this study. Grey violin plots denote probability distributions for the time of the most recent common ancestor of four major clades. (c) Principal component analysis of variants between samples shows geographic clustering. Circular points represent data generated in this study; diamond points represent published genomes from this outbreak.

FIG. 19—Shows maximum likelihood tree and root-to-tip regression. (a) Tips are colored by sample collection location. Bolded tips indicate those generated in this study; all other colored tips are published genomes from the outbreak in the Americas. Grey tips are samples from Zika virus cases in Southeast Asia and the Pacific. (b) Linear regression of root-to-tip divergence on dates supports a molecular clock hypothesis. The substitution rate for the full tree, indicated by the slope of the black regression line, is consistent with rates of Asian lineage ZIKV estimated by molecular clock analyses (Faria et al. 2016). The substitution rate for sequences within the Americas outbreak only, indicated by the slope of the green regression line, is consistent with rates estimated by BEAST [1.04×10⁻³; 95% CI interval (8.54×10⁻⁴, 1.21×10⁻³)] for this data set.

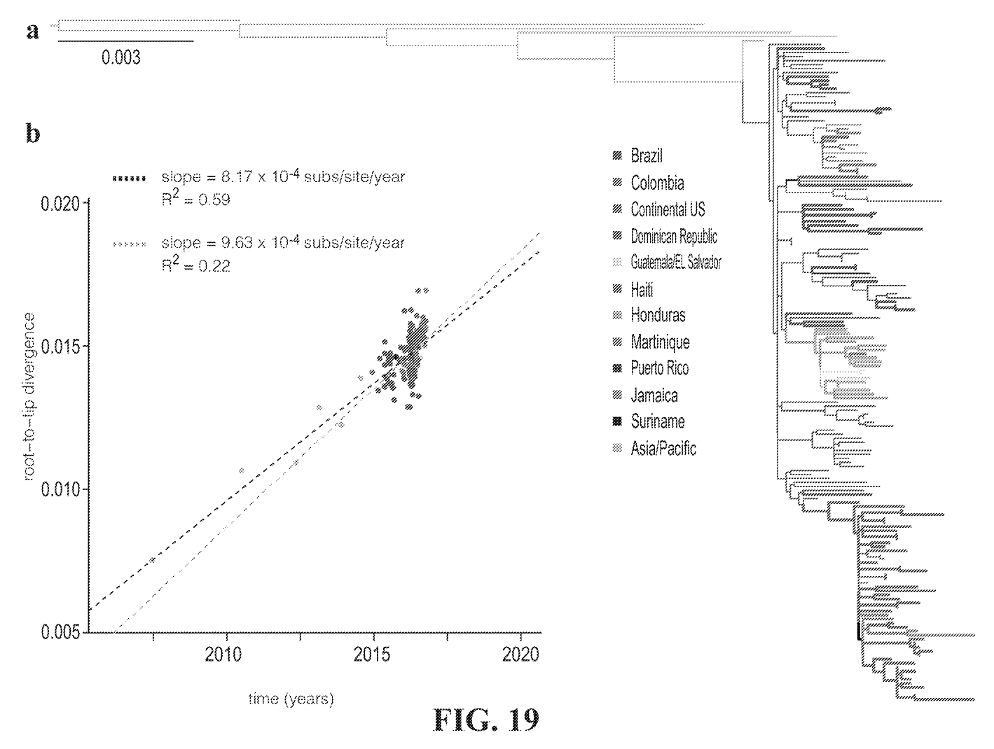

FIG. 20—Shows geographic and gene-level distribution of Zika virus variation. (a) Location of variants in ZIKV genome. The minor allele frequency is the proportion of genomes out of the 100 reported in this study sharing a variant. (b) Phylogenetic distribution of non-synonymous variants that have derived frequency >5% (of the 164 samples in the tree), shown on the branch where the mutation most likely occurred. A white asterisk indicates the variant might be on the next-most ancestral branch (in one case, 2 branches upstream), but the exact location was unclear because of missing data. Square shape denotes a variant occurring at more than one location in the tree. (c) Conservation of the ZIKV envelope gene. Left: non-synonymous variants per genome length for the envelope gene (dark grey) and the rest of the coding region (light grey). Middle: proportion of non-synonymous variants resulting in negative BLOSUM62 scores, which indicate unlikely or extreme substitutions (p<0.038, χ2 test). Right: average of BLOSUM62 scores for non-synonymous variants (p<0.029, 2-sample t-test). Error bars are 95% confidence intervals derived from binomial distributions (left, middle) or Student's t-distributions (right). (d) Constraint in the ZIKV 3′ UTR and transition rates over the ZIKV genome. Error bars are 95% confidence intervals derived from binomial distributions. (e) ZIKV diversity in diagnostic primer and probe regions. Top: locations of published probes (dark blue) and primers (cyan) (Pyke et al., 2014; Lanciotti et al., 2008; Faye et al., 2008; Faye et al., 2013; Balm et al., 2012; Tappe et al., 2014) on ZIKV genome. Bottom: each column represents a nucleotide position in the probe or primer and each row one of the 164 ZIKV genomes on the tree. Cell color indicates that a sample's allele matches the probe/primer sequence (grey), differs from it (red), or has no data for that position (white).

FIG. 21—Shows multiple rounds of Zika hybrid capture. Genome assembly statistics of samples prior to hybrid capture (grey), and after one (blue) or two (red) rounds of hybrid capture. 9 individual libraries (8 unique samples) were sequenced all three ways, had >1 million raw reads in each method, and generated at least one positive assembly. Raw reads from each method were downsampled to the same number of raw reads (8.5 million) before genomes were assembled. (a) Percent of the genome identified, as measured by number of unambiguous bases. (b) Median sequencing depth of Zika genomes, taken over the assembled regions.

FIG. 22—Shows experimental methods to predict sequencing outcome. cDNA concentration of amplicon pools (as measured by Agilent 2200 Tapestation) is highly predictive of amplicon sequencing outcome. On each axis, 1+ primer pool concentration is plotted on a log scale. A sample is considered positive if at least one primer pool concentration is ≥20.8 ng/μL; sensitivity=98.58% and specificity=91.47%.

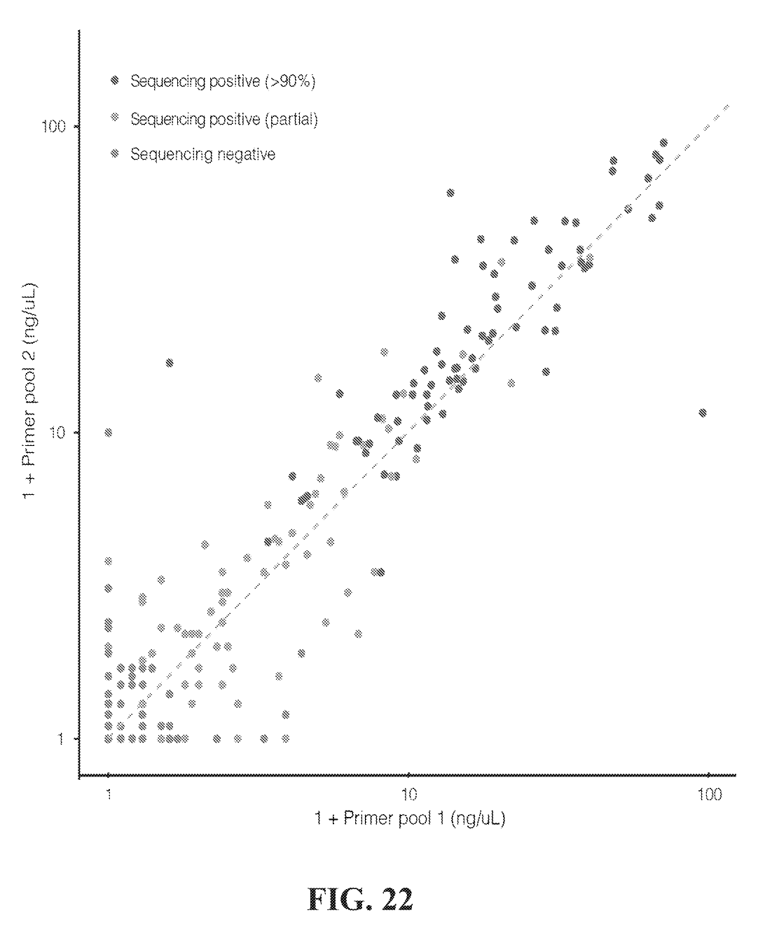

FIG. 23—Analysis of possible predictors of sequencing outcome: the site where a sample was collected, patient gender, patient age, sample type, and days between symptom onset and sample collection (“collection interval”). (a) Prediction of whether a sample passes assembly thresholds by sequencing. Rows show results of likelihood ratio tests on each predictor by omitting the variable from a full model that contains all predictors. Sample site and patient gender improved model fit, but sample type and collection interval did not. (b) Proportion of samples that pass assembly thresholds by sequencing, divided by sample type, across six sample sites. (c) Same as (b), except divided by collection interval. (d) Prediction of the genome fraction identified, using samples passing assembly thresholds. Rows show results of likelihood ratio tests, as in (a). Collection interval improved the model, but sample type did not. (e) Sequencing outcome for each sample, divided by sample type, across six sample sites. (f) Same as (e), except divided by collection interval. Samples collected 7+ days after symptom onset produced, on average, the fewest unambiguous bases, although these observations are based on a limited number of data points. While the sample site variable accounts for differences in cohort composition, the observed effects of gender and collection interval might be due to confounders in composition that span multiple cohorts. These results illustrate the effect of variables on sequencing outcome for the samples in this study; they are not indicative of ZIKV titer more generally. Other studies^{67,68} have analyzed the impact of sample type and collection interval on ZIKV detection, sometimes with differing results.

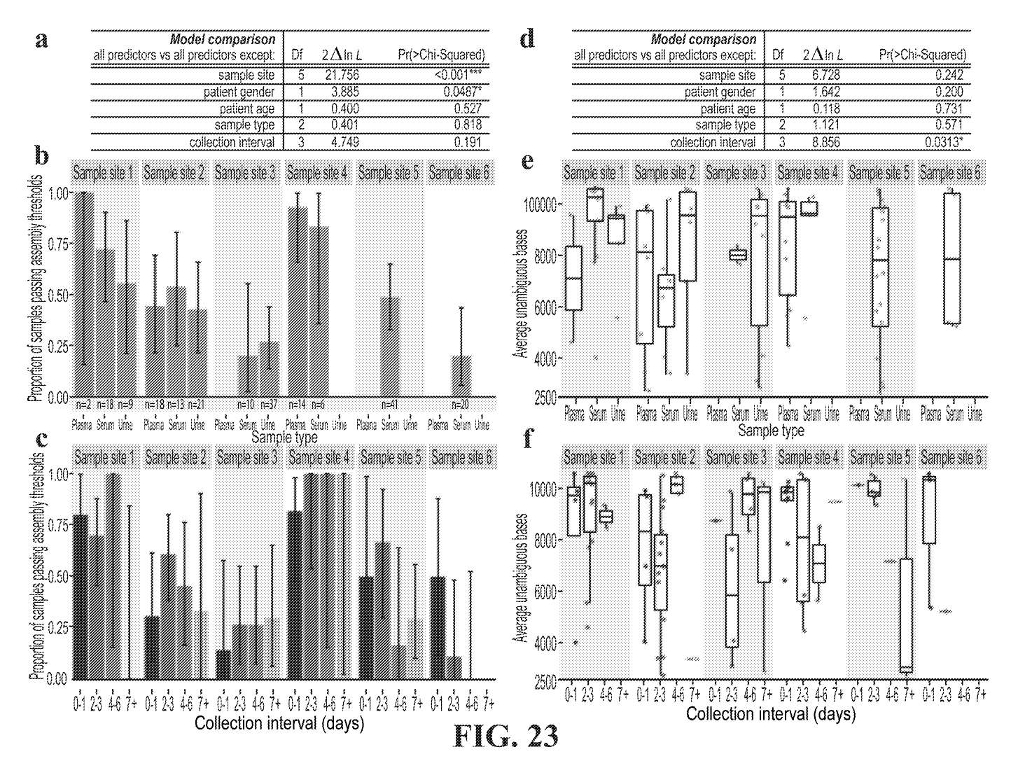

FIG. 24—Maximum likelihood tree and root-to-tip regression. (a) Tips are colored by sample collection location. Labeled tips indicate those generated in this study; all other colored tips are other publicly available genomes from the outbreak in the Americas. Grey tips are samples from ZIKV cases in Southeast Asia and the Pacific. (b) Linear regression of root-to-tip divergence on dates. The substitution rate for the full tree, indicated by the slope of the black regression line, is similar to rates of Asian lineage ZIKV estimated by molecular clock analyses¹². The substitution rate for sequences within the Americas outbreak only, indicated by the slope of the green regression line, is similar to rates estimated by BEAST [1.15×10⁻³; 95% CI (9.78×10⁻⁴, 1.33×10⁻³)] for this data set.

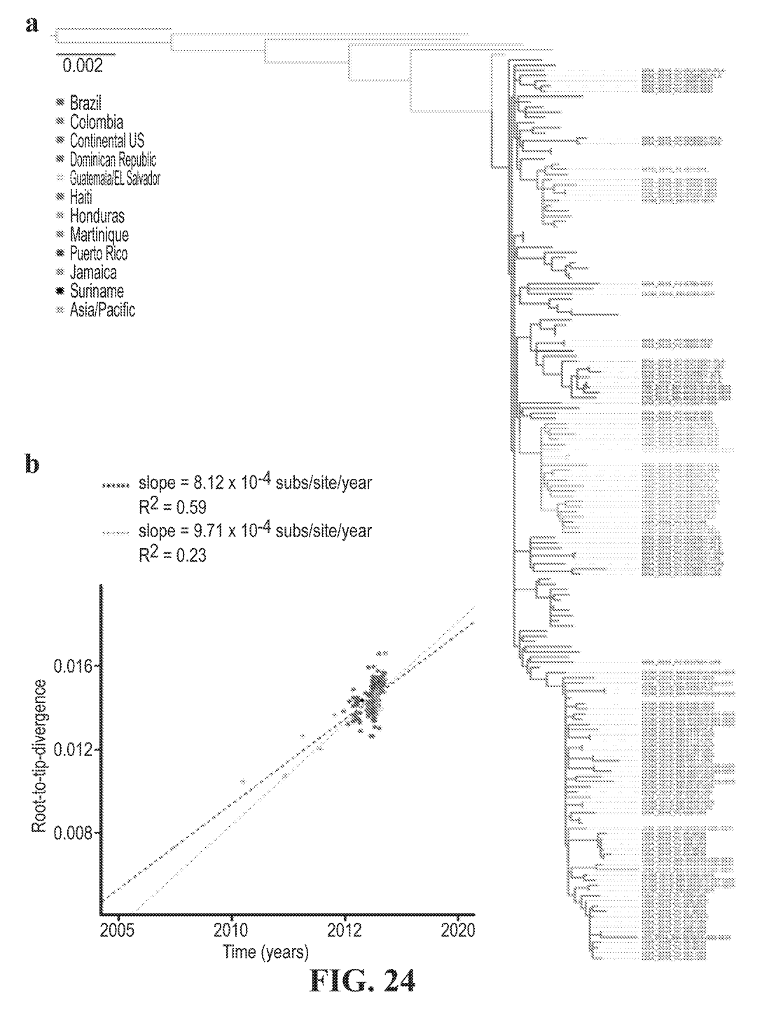

FIG. 25—Substitution rate and tMRCA distributions. (a) Posterior density of the substitution rate. Shown with and without the use of sequences (outgroup) from outside the Americas. (b-e) Posterior density of the date of the most recent common ancestor (MRCA) of sequences in four regions corresponding to those in FIG. 2c. Shown with and without the use of outgroup sequences. The use of outgroup sequences has little effect on estimates of these dates. (f) Posterior density of the date of the MRCA of sequences in a clade consisting of samples from the Caribbean and continental US. Shown with and without the sequence of DOM_2016_MA-WGS 16-020-SER, a sample from the Dominican Republic that has only 3037 unambiguous bases; this was the most ancestral sequence in the clade and its presence affects the tMRCA. In (a-f), all densities are shown as observed with a relaxed clock model and with a strict clock model.

FIG. 26—Substitution rates estimated with BEAST. Substitution rates estimated in three codon positions and non-coding regions (5′ and 3′ UTRs). Transversions are shown in grey and transitions are colored by transition type. Plotted values show the mean of rates calculated at each sampled Markov chain Monte Carlo (MCMC) step of a BEAST run. These calculated rates provide additional evidence for the observed high C-to-T and T-to-C transition rates shown in FIG. 25d.

FIG. 27—cDNA concentration of amplicon primer pools predicts sequencing outcome. cDNA concentration of amplicon pools (as measured by Agilent 2200 Tapestation) was highly predictive of amplicon sequencing outcome. On each axis, 1+ primer pool concentration is plotted on a log scale. Each point demonstrates a technical replicate of a sample and colors denote observed sequencing outcome of the replicate. If a replicate was predicted to be passing when at least one primer pool concentration is ≥0.8 ng/μL, then sensitivity=98.71% and specificity=90.34%. An accurate predictor of sequencing success early in the sample processing workflow can save resources.

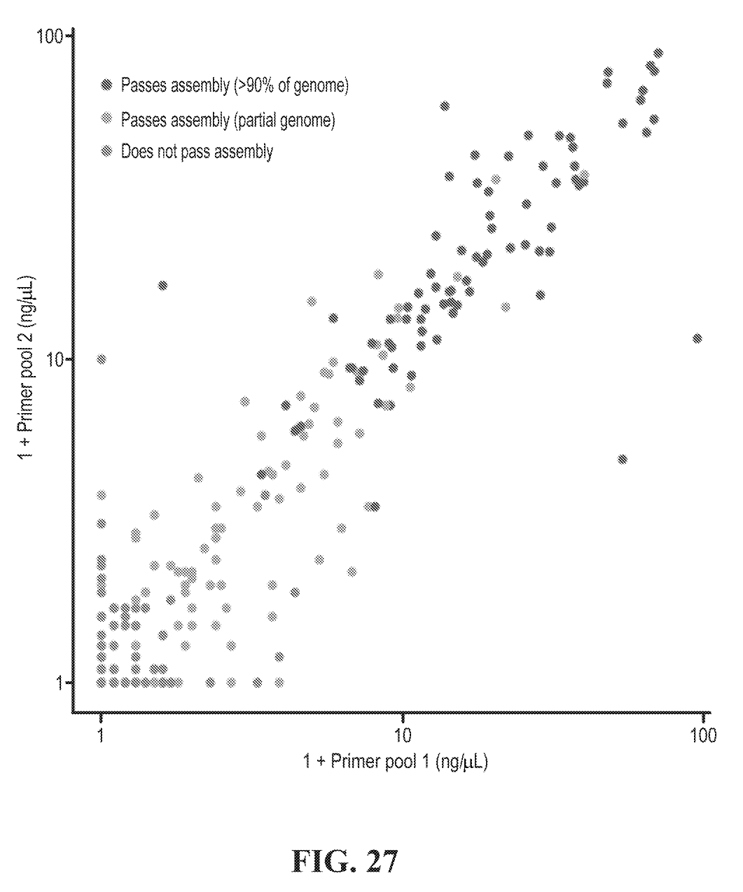

FIG. 28—Evaluating multiple rounds of Zika virus hybrid capture. Genome assembly statistics of samples prior to hybrid capture (grey), and after one (blue) or two (red) rounds of hybrid capture. 9 individual libraries (8 unique samples) were sequenced all three ways, had >1 million raw reads in each method, and generated at least one passing assembly. Raw reads from each method were downsampled to the same number of raw reads (8.5 million) before genomes were assembled. (a) Percent of the genome identified, as measured by number of unambiguous bases. (b) Median sequencing depth of ZIKV genomes, taken over the assembled regions.

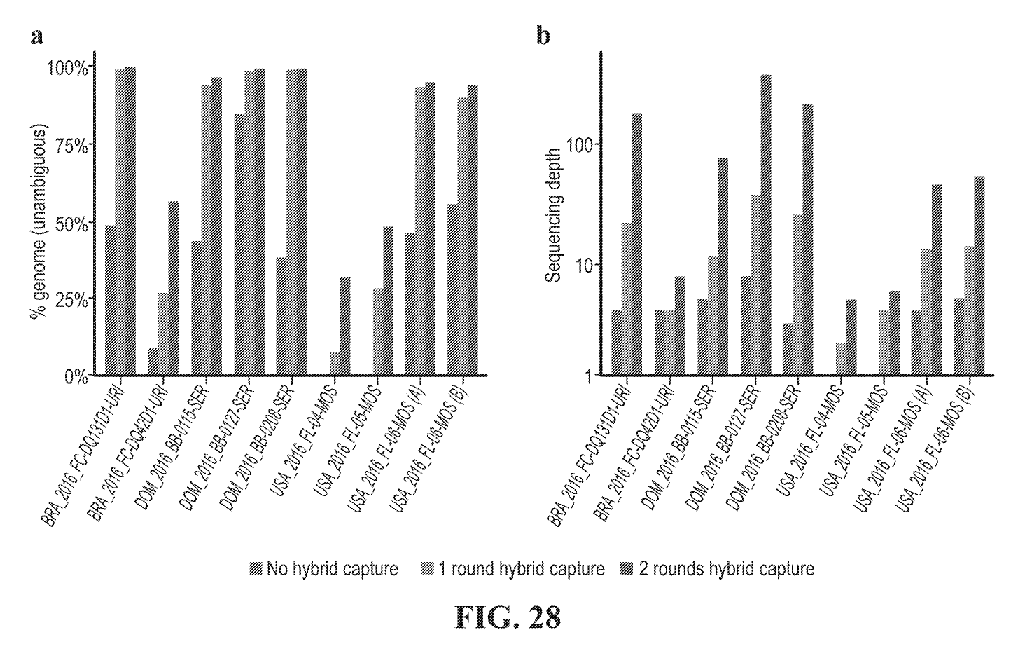

### DETAILED DESCRIPTION OF THE INVENTION

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (RI. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

As used herein the term “hybridize” or “hybridization” refers to ability of oligonucleotides and their analogs to hybridize by hydrogen bonding, which includes Watson-Crick, Hoogsteen, or reversed Hoogsteen hydrogen bonding, between complementary bases, Generally nucleic acid consists of nitrogenous bases that are either either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or it's analog) and the DNA or RNA target. The oligonucleotide or oligonucleotide analog need not be 100% complementary to its target sequence to be specifically hybridizable. An oligonucleotide or analog is specifically hybridizable when there is a sufficient degree of complementarity to avoid non-specific binding of the oligonucleotide or analog to non-target sequences under conditions where specific binding is desired. Such binding is referred to as specific hybridization.

The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations. The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166+1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (i.e., 15+20*100=75).

The term “amplification” refers to methods to increase the number of copies of a nucleic acid molecule. The resulting amplification products are typically called “amplicons.” Amplification of a nucleic acid molecule (such as a DNA or RNA molecule) refers to use of a technique that increases the number of copies of a nucleic acid molecule (including fragments). In some examples, an amplicon is a nucleic acid from a cell, or acellular system, such as mRNA or DNA that has been amplified.

An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); real-time reverse transcriptase PCR (rt RT-PCR); nested PCR; strand displacement amplification (see U.S. Pat. No. 5,744,311); transcription-free isothermal amplification (see U.S. Pat. No. 6,033,881, repair chain reaction amplification (see WO 90/01069); ligase chain reaction amplification (see European patent publication EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others

The term “primer” or “primers” refers to short nucleic acid molecules, such as a DNA oligonucleotide, for example sequences of at least 15 nucleotides, which can be annealed to a complementary nucleic acid molecule by nucleic acid hybridization to form a hybrid between the primer and the nucleic acid strand. A primer can be extended along the nucleic acid molecule by a polymerase enzyme. Therefore, primers can be used to amplify a nucleic acid molecule, wherein the sequence of the primer is specific for the nucleic acid molecule, for example so that the primer will hybridize to the nucleic acid molecule under very high stringency hybridization conditions. The specificity of a primer increases with its length. Thus, for example, a primer that includes 30 consecutive nucleotides will anneal to a sequence with a higher specificity than a corresponding primer of only 15 nucleotides. Thus, to obtain greater specificity, probes and primers can be selected that include at least 15, 20, 25, 30, 35, 40, 45, 50 or more consecutive nucleotides.

In particular examples, a primer is at least 15 nucleotides in length, such as at least 15 contiguous nucleotides complementary to a nucleic acid molecule. Particular lengths of primers that can be used to practice the methods of the present disclosure, include primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or more contiguous nucleotides complementary to the target nucleic acid molecule to be amplified, such as a primer of 15-60 nucleotides, 15-50 nucleotides, or 15-30 nucleotides.

Primer pairs can be used for amplification of a nucleic acid sequence, for example, by PCR, real-time PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction. PCR primer pairs can be derived from a known sequence, for example, by using computer programs intended for that purpose such as Primer (Version 0.5, © 1991, Whitehead Institute for Biomedical Research, Cambridge, Mass.).

The term “probe” refers to an isolated nucleic acid capable of hybridizing to a specific nucleic acid (such as a nucleic acid barcode or target nucleic acid). A detectable label or reporter molecule can be attached to a probe. Typical labels include radioactive isotopes, enzyme substrates, co-factors, ligands, chemiluminescent or fluorescent agents, haptens, and enzymes. In some example, a probe is used to isolate and/or detect a specific nucleic acid.

Methods for labeling and guidance in the choice of labels appropriate for various purposes are discussed, for example, in Sambrook et al., Molecular Cloning: A Laboratory Manual, Cold Spring Harbor Laboratory Press (1989) and Ausubel et al., Current Protocols in Molecular Biology, Greene Publishing Associates and Wiley-Intersciences (1987).

Probes are generally about 15 nucleotides in length to about 160 nucleotides in length, such as 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160 contiguous nucleotides complementary to the specific nucleic acid molecule, such as 50-140 nucleotides, 75-150 nucleotides, 60-70 nucleotides, 30-130 nucleotides, 20-60 nucleotides, 20-50 nucleotides, 20-40 nucleotides, or 20-30 nucleotides.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Future pandemics threaten human progress and must be detected early. The goal of the present study was to achieve a sustainable, rapid-response surveillance system to detect infectious disease outbreaks as soon as they appear. To do so, vast improvement is needed in both diagnostic tools and the human resources to deploy them. The present invention therefore relates to developing rapid pathogen sequencing for comprehensive microbial detection.

Rapid advances in DNA amplification and detection technology provide an unprecedented capability to identify and characterize pathogens, and will soon enable comprehensive and unbiased pathogen surveillance for early detection and prevention of future epidemics. However, realizing its full potential for infectious disease surveillance and clinical diagnosis present additional challenges, which require further investment and focused effort.

The present invention relates to a method for generating primers and/or probes for use in analyzing a sample which may comprise a pathogen target sequence comprising providing a set of input genomic sequence to one or more target pathogens, generating a set of target sequences from the set of input genomic sequences, identifying one or more highly conserved target sequences, and generating one or more primers, one or more probes, or a primer pair and probe combination based on the one or more conserved target sequences.

In certain example embodiments, the methods for identifying highly conserved sequences between genomic sequences of one or more target pathogens may comprise use a set cover solving process. The set cover solving process may identify the minimal number of probes needed to cover one or more conserved target sequence. Set cover approaches have been used previously to identify primers and/or microarray probes, typically in the 20 to 50 base pair range. See, e.g. Pearson et al., cs.virginia.edu/-robins/papers/primers_dam11_final.pdf., Jabado et al. Nucleic Acids Res. 2006 34(22):6605-11, Jabado et al. Nucleic Acids Res. 2008, 36(1):e3 doi10.1093/nar/gkm1106, Duitama et al. Nucleic Acids Res. 2009, 37(8):2483-2492, Phillippy et al. BMC Bioinformatics. 2009, 10:293 doi:10.1186/1471-2105-10-293. However, such approaches generally involved treating each primer/probe as k-mers and searching for exact matches or allowing for inexact matches using suffix arrays. In addition, the methods generally take a binary approach to detecting hybridization by selecting primers or probes such that each input sequence only needs to be bound by one primer or probe and the position of this binding along the sequence is irrelevant. Alternative methods may divide a target genome into pre-defined windows and effectively treat each window as a separate input sequence under the binary approach—i.e., they determine whether a given primer or probe binds within each window and require that all of the windows be bound by the state of some primer or probe. Effectively, these approaches treat each element of the “universe” in the set cover problem as being either an entire input sequence or a pre-defined window of an input sequence, and each element is considered “covered” if the start of a probe binds within the element. These approaches limit the fluidity to which different primer or probe designs are allowed to cover a given target sequence.

In contrast, the methods disclosed herein take a pan-target sequence approach capable of defining a probe set that can identify and increase the sensitivity of pathogen detection assays by identifying highly conserved regions shared among multiple variants of the same pathogen or across different pathogens. For example, the methods disclosed herein may be used to identify all variants of a given virus, or multiple different viruses in a single assay. In addition, the methods disclosed herein may be used to detect all variants of a parasitic pathogen, or multiple different parasitic pathogens in a single assay. Further, the methods disclosed herein treat each element of the “universe” in the set cover problem as being a nucleotide of a target sequence, and each element is considered “covered” as long as a probe binds to some segment of a target genome that includes the element. Instead of the binary approach of previous methods, the methods disclosed herein better model how a probe, and in particular larger probes, may hybridize to a target sequence. Rather than only asking if a given sequence does or does not bind to a given window, embodiments disclosed herein first determine a hybridization pattern—i.e., where a given probe binds to a target sequence or target sequences—and then determines from those hybridization patterns of highly conserved sequences with low to now variability between sequences. These hybridization patterns may be determined by defining certain parameters that minimize a loss function, thereby enabling identification of minimal primer and probes sets in a way that allows parameter to vary for each species, e.g., to reflect the diversity of each species, as well as in a computationally efficient manner that cannot be achieved using a straightforward application of a set cover solution, such as those previously applied in the primer and microarray probe design context.

A primer in accordance with the invention may be an oligonucleotide for example deoxyribonucleic acid (DNA), ribonucleic acid (RNA), peptide nucleic acid (PNA), or other non-naturally occurring nucleic acid. A probe, a candidate probe, or a selected probe may be a nucleic acid sequence, the nucleic acid being, for example, deoxyribonucleic acid (DNA), ribonucleic acid (RNA), peptide nucleic acid (PNA), or other non-naturally occurring nucleic acid.

A sample as described herein may be a biological sample, for example a blood, buccal, cell, cerebrospinal fluid, mucus, saliva, semen, tissue, tumor, feces, urine, and/or vaginal sample. A sample may be obtained from an animal, a plant, or a fungus. The animal may be a mammal. The mammal may be a primate. The primate may be a human. In other embodiments, the sample may be an environmental sample, such as water, soil, or a surface, such as an industrial or medical surface.

As used herein, “target sequence” is intended to designate either one target sequence or more than one target sequence, i.e., any sequence of interest at which the analysis is aimed. Thus, the sample may comprise more than one target sequence and preferably a plurality of target sequences. The target sequence may be a nucleotide sequence. The nucleotide sequence may be a DNA sequence, a RNA sequence, or a mixture thereof.

The set of target sequences may comprise obtaining a nucleic acid array (e.g., a microarray chip) and synthesizing a set of synthetic oligonucleotides, and removing the oligonucleotides from the microarray (e.g., by cleavage or elution) to produce a set of target sequences. Synthesis of oligonucleotides in an array format (e.g., chip) permits synthesis of a large number of sequences simultaneously, thereby providing a set of target sequences for the methods of selection. The array synthesis also has the advantages of being customizable and capable of producing long oligonucleotides.

The target sequences may be prepared from the whole genome of the target pathogen, for example, where the target sequences are prepared by a method that includes fragmenting genomic DNA of the target pathogen (e.g., where the fragmented target sequences are end-labeled with oligonucleotide sequences suitable for PCR amplification or where the target sequences are prepared by a method including attaching an RNA promoter sequence to the genomic DNA fragments and preparing the target sequences by transcribing (e.g., using biotinylated ribonucleotides) the DNA fragments into RNA. The target sequences may be prepared from specific regions of the target organism genome (e.g., are prepared synthetically). In certain embodiments, the target sequences are labeled with an affinity tag. In certain example embodiments, the affinity tag is biotin, a hapten, or an affinity tag, or the target sequences are generated using biotinylated primers, e.g., where the target sequences are generated by nick-translation labeling of purified target organism DNA with biotinylated deoxynucleotides. In cases where the target sequences are biotinylated, the target DNA can be captured using a streptavidin molecule attached to a solid phase. The target sequences may be appended by adapter sequences suitable for PCR amplification, sequencing, or RNA transcription. The target sequences may include a RNA promoter or are RNA molecules prepared from DNA containing an RNA promoter (e.g., a T7 RNA promoter).

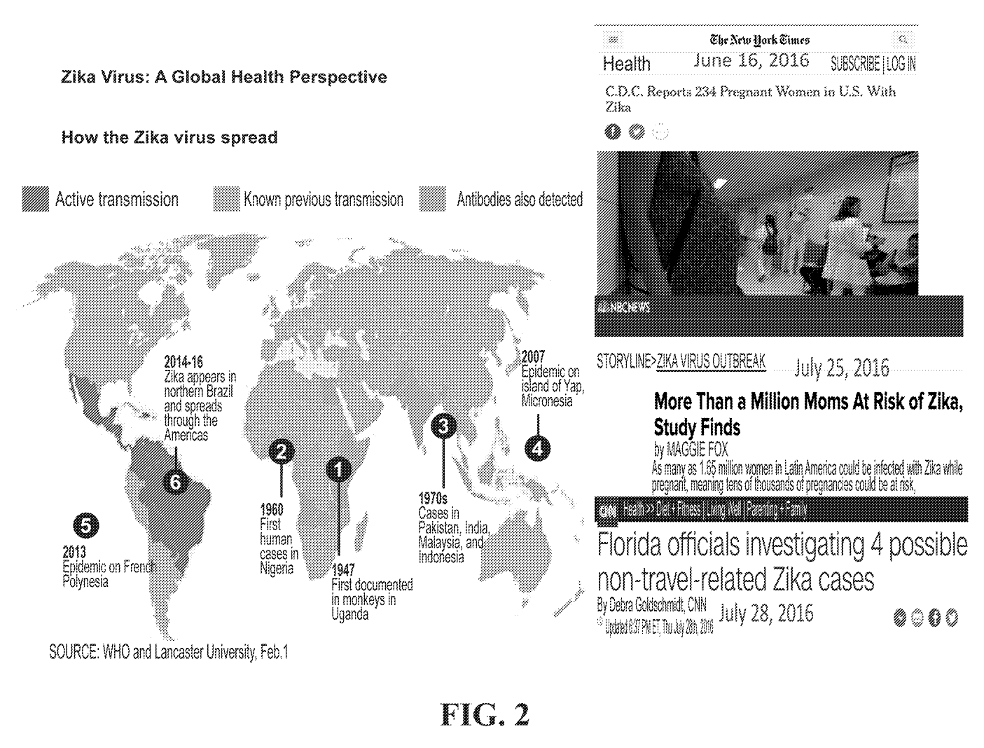

Constructing the target sequence may comprise fragmenting the reference genomic sequences into fragments of equal size that overlap one another, so that the overlap between two fragments is half the size of the fragment, for example a 2× tiling as illustrated in FIG. 2.

As used herein, “individual hybridization pattern” is intended to designate the coverage capacity of one probe, i.e., the portion of the reference sequences to which the target sequence is capable of aligning or hybridizing to. More generally, when used with respect to a plurality of target sequence, “hybridization pattern” is intended to designate the collective coverage capacity of the plurality of target sequences, i.e. the collection of subsequences of the reference sequence which at least one of the target sequences of the plurality of target sequences is capable of hybridizing or aligning to or to which at least one of the target sequences is redundant once aligned to the reference genomic sequence.

A set cover solving process may be used to identify target sequences that are highly conserved among the input genomic sequences. A set cover solving process may refer to any process that approximates the solution to the set cover problem or a problem equivalent to the set cover problem (see, e.g., Introduction to Algorithms (mitpress.mit.edu/books/introduction-algorithms) and cc.gatech.edu/fac/Vijay.Vazirani/book.pdf). A set cover problem may be described as follows: given a set of elements {1, 2 . . . i . . . m}, called the universe U, and a collection S of n subsets whose union covers the universe, the set cover problem is to identify the smallest set of subsets whose union equals the universe.

As used herein, “reference genomic sequence” is intended to encompass the singular and the plural. As such, when referring to a reference sequence, the cases where more than one reference sequence is also contemplated. Preferably, the reference sequence is a plurality of reference sequences, the number of which may be over 30; 50; 70; 100; 200; 300; 500; 1,000 and above. In certain example embodiments, the reference sequence is a genomic sequence. In certain example embodiments, the reference sequence is a plurality of genomic sequences. In certain example embodiments, the reference sequence is a plurality of genomic sequences from the same species or viral strain. In certain other example embodiments, the reference sequence is a plurality of genomic sequences from different species or viral strains.

In one embodiment, the reference sequence may be a collection of genomes of one type of virus, wherein the genomes collectively form a universe of elements that are the nucleotides (position within the genomes being considered as differentiating nucleotides of the same type). In another embodiment, each genome may make up one universe so that the problem as a whole becomes a multi-universe problem. Multi-universe may be a unique generalization of the set cover problem. In this instance, separate universes may be helpful for thinking about partial set cover, so that this way, a partial cover yields a desired partial coverage of each genome (i.e., each universe). If the problem is imagined as being composed of a single universe, thinking about partial coverage may be considered as covering a desired fraction of the concatenation of all the genomes, rather than a desired fraction of each genome.

If X designates a genome and y designates a position within the corresponding genome, an element of the universe can be represented by (X, y), which is understood as the nucleotide in position y in genome X. Candidate probes are obtaining by fragmenting the collection of genomes. The individual hybridization patterns are subsets of the universe. The individual hybridization pattern of a candidate probe of length L can be represented as {(A, ai), (A, ai+1) . . . (A, ai+L), (A, aj), (A, aj+1) . . . (A, aj+L), (B, bi), (B, bi+1) . . . (B, bi+L) . . . }, otherwise represented as {A:(ai . . . ai+L), (aj . . . aj+L); B:(b1 . . . b1+L) . . . } (subset covering nucleotides in position ai to ai+L and aj to aj+L in genome A, nucleotides in position bi to bi+L in genome B . . . ).

In certain example embodiments, the target genomic sequences are viral genomic sequences. The viral sequences may be variants of the same viral strain, different viruses, or a combination thereof. A hybridization pattern is determined for the target sequences. To model a hybridization pattern, a number of different parameters may be defined to determine whether a given target sequence is considered to hybridize to a given portion of a reference genomic sequence. In addition, a percent of coverage parameter may be set to define the percent of the target sequence that should be covered by the probe set. This value may range from a fraction of a percent to 100% of the genome. In certain example embodiments, this may range from 0.01% to 10%, 1% to 5%, 1% to 10%, 1% to 15%, 1% to 20%, 1% to 25%, or the like.

In certain example embodiments, a number of mismatch parameters is defined. The number of mismatches defines a number of mismatches that may be present between a probe and a given portion of a target sequence. This value may range from 0 to 10 base pairs.

In certain example embodiments, another parameter, called the “island of exact match” substring, may be used to model hybridization between a probe and nucleic acid fragment. Let its value be x. When determining whether a probe covers a sequence, a value is set that defines a stretch of at least x bp in the probe that exactly matches (i.e., with no mismatches) a stretch of a target sequence. Along with the other parameters, this is applied as a filter to decide whether a probe should be deemed as hybridizing to a portion of a target sequence. The value may vary, but is usually set to be 30 bp. Setting its value to 0 would effectively remove this filter when determining hybridization patterns.

In certain other example embodiments, a longest common substring parameter may be set. This parameter defines that a probe only hybridizes if the longest common substring up to a certain amount of mismatches is at least that parameter. For example, if the parameter is set to 80 base pair with 3 mismatches, then a probe will still be considered to hybridized to a portion of a target sequence if there is string of 80 base pairs that match the target sequence, even if within that stretch, there are up to 3 mismatches. So, an 80-base-pair string that matches except for two mismatches would be considered to be hybridized, but an 80-base-pair string that matches except for 4 mismatches would not be considered to hybridize. This parameter may range from a string of 20 to 175 base pairs with anywhere from 0 to 9 mismatches in that string.

In certain other example embodiments, an overhang or cover extension parameter may be set. This parameter indicates that once a probe is found to hybridize, that probe will be considered to cover, or account for, X additional base pairs upstream and downstream of where the probe has bound. This parameter allows the number of total probes required to be reduced further because it will be understood that a probe, e.g., 100 base pairs, will not only account for the 100 base pairs portion it directly binds to, but may be reliably considered to capture a fragment that is at least 50 base pairs longer than the 100 base pair string. This parameter may vary between 0 and 200. In certain example embodiments, this parameter is set to 50.

This can be used, for example, in sequencing genomes of a virus for which a collection of genomes is available from previous studies, such as Zika virus. The collection of available genomes from previous studies is taken as reference target. One aim may be the study and monitoring of the evolution of the virus, for example throughout an outbreak, in order to determine proper actions to be taken for containing the outbreak and stopping it by sequencing regularly, if not systematically, the genome of the virus that infects a patient known to have contracted it.

The set cover solving process may be a weighted set cover solving process, i.e., each of the individual hybridization patterns is allocated a weight.

For example, a lower weight is allocated to those individual hybridization patterns that correspond to candidate target sequences that are specific to the reference sequence and a higher weight is allocated to those individual hybridization patterns that correspond to target sequences that are not specific to the reference sequence. Thus, the method may further comprise determining the specificity of each target sequence with regard to the reference sequence. For example, determining the stringency of hybridization may be indicative of the specificity of the target sequence. The higher weight is determined based on when a target sequence hybridizes to some other reference sequence (not a target). Another mismatch parameter may be utilized when assigning higher weights, which is usually a looser and more tolerant value. For example, there may be a mismatch parameter with a value of 3 for determining whether a target sequence hybridizes to a region of a reference sequence, but a separate tolerant mismatch parameter with a value of 10 for determining whether a probe hits a blacklisted sequence or more than one virus type in identification. The reason is desired increased sensitivity in determining these kinds of hits and more specificity in determining where target sequence cover reference sequences.

The weighted set cover solving process makes it possible to reduce substantially, if not dramatically, the number of selected target sequences that are highly conserved among reference sequences.

In certain example embodiments, the reference sequence forms a universe of elements that are the nucleotides (positions within the genomes being considered as differentiating nucleotides of the same type). If X designates the target sequence and y designates a position within the corresponding genome, an element of the universe can be represented by (X, y), which is understood as the nucleotide in position y in the target sequence X, or simply (y) because all y belongs to the same target sequence. Target sequences are obtained by fragmenting the reference sequence. It is then determined which target sequences are specific to the reference sequence and which are not. The individual hybridization patterns are subsets of the universe. The individual hybridization pattern of a target sequence of length L and which is specific to the reference sequence can be represented as (w, {(ai), (ai+1) . . . (ai+L), (aj), (aj+1) . . . (aj+L)}), otherwise represented as (w, {(ai . . . ai+L), (aj . . . aj+L)}) (subset covering nucleotides in position ai to ai+L . . . and aj to aj+L to which a weight w is given). The individual hybridization pattern of a target sequence of length L and which is not specific to the reference sequence would be represented in the same manner but will receive weight W instead, wherein W>w, preferably W>>w, more preferably W is infinity and w is 1.

If the reference sequence is a collection of reference sequences, then the individual hybridization pattern of a candidate probe of length L and which is specific to the reference sequence can be represented as (V, {(A, ai), (A, ai+1) . . . (A, ai+L), (A, aj), (A, aj+1) . . . (A, aj+L), (B, bi), (B, bi+1) . . . (B, bi+L) . . . }), otherwise represented as (V, {A:(ai . . . ai+L), (aj . . . aj+L); B:(bi . . . bi+L) . . . }) (subset covering nucleotides in position ai to ai+L and aj to aj+L in genome A, nucleotides in position bi to bi+L in genome B . . . to which a weight V is given).

Allocating the same weight to all the individual hybridization patterns amounts to an un-weighted set cover solving process, in other words, a set cover solving process without allocation of any weight, such as described above. Both weighted set cover solving process and un-weighted set cover solving process are contemplated by the invention.

A higher number of allowed mismatches for the weighted than for the un-weighted set cover solving process may be used, which is considered to be a separate, more tolerant parameter choice—in addition to the regular mismatch parameter that would be used (in the un-weighted problem) for determining hybridizations to target sequences. But, if the higher number does not replace the lower number, it is an additional parameter.

One example of a process that approximates the solution to the set cover problem is the greedy method. The greedy method is an iterative method wherein at each iteration, the solution that appears the best is chosen. When applied to the set cover problem at each iteration, the subset with the widest coverage of the yet uncovered universe is selected and the elements covered by the subset with the widest coverage are deleted from the yet uncovered universe. This is repeated until all the selected subsets collectively cover the entire universe, in other words, the yet uncovered universe, is empty.

Within the scope of the invention, this means that, at each iteration, the target sequence with the widest individual hybridization pattern within yet uncovered portions of the reference sequence is selected as one of the selected target sequences. The selection is repeated among the remaining target sequences until the selected probes collectively have a hybridization pattern that equals the desired coverage percentage of the reference sequences.

The method may further comprise minimizing a loss function depending on overhang parameters and mismatch parameters (or any parameters that alters the number of output probes) such that the total number of selected probes is no higher than a threshold number to provide input parameters to the set cover solving process. An overhang parameter (“cover extension”) determines the number of nucleotides of one or both ends of a target sequence or a fragment thereof that remain unpaired once the target sequence or the fragment thereof hybridizes a selected probe. The higher the overhang parameter is, the lower the number of selected probes output by the set cover solving process. The value of the overhang parameters can range from 0 to 200 bp, and any sub-range therein. A mismatch parameter is the acceptable number of mismatches between a selected probe and the target sequence or the fragment thereof. The higher the mismatch parameter is, the lower the number of selected probes. In certain example embodiments, the mismatch parameter may have a range from 0 to 9.

In the case of a plurality of target sequence types, one overhang parameter and one mismatch parameter is assigned to each reference sequence or types thereof. The values of the overhang and mismatch parameters may be indicative of the diversity of the reference sequence, especially when selecting these parameters under the constraint of having a fixed number of probes.

The loss function is constructed so that the higher the value of the overhang parameter, the higher the value of the loss function, and the higher the value of the mismatch parameter, the higher the value of the loss function.

The use of a constraint while minimizing the loss function ensures that the number of selected probes remains lower than a reasonable amount, depending on the application of the selected probes.

The selected primers or probe can be used in a composition form, as part of a kit or a system for detection of pathogen nucleic acids sequence. The kit may comprise primers and/or probes generated from the identified target sequences, e.g., in a composition form, and a solid phase operably linked to the selected probes. The system may comprise the selected probes, i.e., in a composition form; a sample containing DNA of said target organism and the non-specific DNA; and a solid phase operably connected to the selected probes.

The solid phase may be a chip or beads. The selected probes may further comprise an adapter, for example a label. Each selected probe may comprise two adapters. Preferably, a first adapter is alternated with a second adapter.

As described in aspects of the invention, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

Sequence homologies may be generated by any of a number of computer programs known in the art, for example BLAST or FASTA, etc. A suitable computer program for carrying out such an alignment is the GCG Wisconsin Bestfit package (University of Wisconsin, U.S.A; Devereux et al., 1984, Nucleic Acids Research 12:387). Examples of other software than may perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program. % homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues. Although this is a very simple and consistent method, it fails to take into consideration that, for example, in an otherwise identical pair of sequences, one insertion or deletion may cause the following amino acid residues to be put out of alignment, thus potentially resulting in a large reduction in % homology when a global alignment is performed. Consequently, most sequence comparison methods are designed to produce optimal alignments that take into consideration possible insertions and deletions without unduly penalizing the overall homology or identity score. This is achieved by inserting “gaps” in the sequence alignment to try to maximize local homology or identity. However, these more complex methods assign “gap penalties” to each gap that occurs in the alignment so that, for the same number of identical amino acids, a sequence alignment with as few gaps as possible—reflecting higher relatedness between the two compared sequences—may achieve a higher score than one with many gaps. “Affinity gap costs” are typically used that charge a relatively high cost for the existence of a gap and a smaller penalty for each subsequent residue in the gap. This is the most commonly used gap scoring system. High gap penalties may, of course, produce optimized alignments with fewer gaps. Most alignment programs allow the gap penalties to be modified. However, it is preferred to use the default values when using such software for sequence comparisons. For example, when using the GCG Wisconsin Bestfit package, the default gap penalty for amino acid sequences is −12 for a gap and −4 for each extension. Calculation of maximum % homology, therefore, first requires the production of an optimal alignment, taking into consideration gap penalties. A suitable computer program for carrying out such an alignment is the GCG Wisconsin Bestfit package (Devereux et al., 1984 Nuc. Acids Research 12 p 387). Examples of other software than may perform sequence comparisons include, but are not limited to, the BLAST package (see Ausubel et al., 1999 Short Protocols in Molecular Biology, 4th Ed. —Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60). However, for some applications, it is preferred to use the GCG Bestfit program. A new tool, called BLAST 2 Sequences is also available for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health). Although the final % homology may be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table, if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins D G & Sharp P M (1988), Gene 73(1), 237-244). Once the software has produced an optimal alignment, it is possible to calculate % homology, preferably % sequence identity. The software typically does this as part of the sequence comparison and generates a numerical result.

Embodiments of the invention include sequences (both polynucleotide or polypeptide) which may comprise homologous substitution (substitution and replacement are both used herein to mean the interchange of an existing amino acid residue or nucleotide, with an alternative residue or nucleotide) that may occur i.e., like-for-like substitution in the case of amino acids, such as basic for basic, acidic for acidic, polar for polar, etc. Non-homologous substitution may also occur i.e., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Hybridization can be performed under conditions of various stringency. Suitable hybridization conditions for the practice of the present invention are such that the recognition interaction between the probe and sequences associated with a signaling biochemical pathway is both sufficiently specific and sufficiently stable. Conditions that increase the stringency of a hybridization reaction are widely known and published in the art. See, for example, (Sambrook, et al., (1989); Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present invention include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, ß-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

Examples of the labeling substance which may be employed include labeling substances known to those skilled in the art, such as fluorescent dyes, enzymes, coenzymes, chemiluminescent substances, and radioactive substances. Specific examples include radioisotopes (e.g., 32P, 14C, 125I, 3H, and 131I), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added.

Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-di sulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

The fluorescent label may be a fluorescent protein, such as blue fluorescent protein, cyan fluorescent protein, green fluorescent protein, red fluorescent protein, yellow fluorescent protein or any photoconvertible protein. Colorimetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code.

In an advantageous embodiment, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

The oligonucleotide tags may be detectable by virtue of their nucleotide sequence, or by virtue of a non-nucleic acid detectable moiety that is attached to the oligonucleotide such as, but not limited to, a fluorophore, or by virtue of a combination of their nucleotide sequence and the non-nucleic acid detectable moiety.

In some embodiments, a detectable oligonucleotide tag may comprise one or more non-oligonucleotide detectable moieties. Examples of detectable moieties may include, but are not limited to, fluorophores, microparticles, including quantum dots (Empodocles, et al., Nature 399:126-130, 1999), gold nanoparticles (Reichert et al., Anal. Chem. 72:6025-6029, 2000), biotin, DNP (dinitrophenyl), fucose, digoxigenin, haptens, and other detectable moieties known to those skilled in the art. In some embodiments, the detectable moieties may be quantum dots. Methods for detecting such moieties are described herein and/or are known in the art.

Thus, detectable oligonucleotide tags may be, but are not limited to, oligonucleotides that may comprise unique nucleotide sequences, oligonucleotides that may comprise detectable moieties, and oligonucleotides that may comprise both unique nucleotide sequences and detectable moieties.

A unique label may be produced by sequentially attaching two or more detectable oligonucleotide tags to each other. The detectable tags may be present or provided in a plurality of detectable tags. The same or a different plurality of tags may be used as the source of each detectable tag may be part of a unique label. In other words, a plurality of tags may be subdivided into subsets and single subsets may be used as the source for each tag.

A unique nucleotide sequence may be a nucleotide sequence that is different (and thus distinguishable) from the sequence of each detectable oligonucleotide tag in a plurality of detectable oligonucleotide tags. A unique nucleotide sequence may also be a nucleotide sequence that is different (and thus distinguishable) from the sequence of each detectable oligonucleotide tag in a first plurality of detectable oligonucleotide tags but identical to the sequence of at least one detectable oligonucleotide tag in a second plurality of detectable oligonucleotide tags. A unique sequence may differ from other sequences by multiple bases (or base pairs). The multiple bases may be contiguous or non-contiguous. Methods for obtaining nucleotide sequences (e.g., sequencing methods) are described herein and/or are known in the art.

In some embodiments, detectable oligonucleotide tags comprise one or more of a ligation sequence, a priming sequence, a capture sequence, and a unique sequence (optionally referred to herein as an index sequence). A ligation sequence is a sequence complementary to a second nucleotide sequence which allows for ligation of the detectable oligonucleotide tag to another entity which may comprise the second nucleotide sequence, e.g., another detectable oligonucleotide tag or an oligonucleotide adapter. A priming sequence is a sequence complementary to a primer, e.g., an oligonucleotide primer used for an amplification reaction such as but not limited to PCR. A capture sequence is a sequence capable of being bound by a capture entity. A capture entity may be an oligonucleotide which may comprise a nucleotide sequence complementary to a capture sequence, e.g. a second detectable oligonucleotide tag. A capture entity may also be any other entity capable of binding to the capture sequence, e.g. an antibody, hapten, or peptide. An index sequence is a sequence that may comprise a unique nucleotide sequence and/or a detectable moiety as described above.

The present invention also relates to a computer system involved in carrying out the methods of the invention relating to both computations and sequencing.

A computer system (or digital device) may be used to receive, transmit, display and/or store results, analyze the results, and/or produce a report of the results and analysis. A computer system may be understood as a logical apparatus that can read instructions from media (e.g., software) and/or network port (e.g., from the internet), which can optionally be connected to a server having fixed media. A computer system may comprise one or more of a CPU, disk drives, input devices such as keyboard and/or mouse, and a display (e.g., a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. The receiver can be, but is not limited to an individual, or electronic system (e.g., one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine-readable medium which may comprise computer-executable code may take many forms, including, but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc., shown in the drawings. Volatile storage media include dynamic memory, such as main memory of such a computer platform. Tangible transmission media include coaxial cables, copper wire, and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

The present invention also contemplates multiplex assays. The present invention is especially well suited for multiplex assays. For example, the invention encompasses use of a SureSelectXT, SureSelectXT2 and SureSelectQXT Target Enrichment System for Illumina Multiplexed Sequencing developed by Agilent Technologies (see, e.g., agilent.com/genomics/protocolvideos), a SeqCap EZ kit developed by Roche NimbleGen, a TruSeq® Enrichment Kit developed by Illumina and other hybridization-based target enrichment methods and kits that add sample-specific sequence tags either before or after the enrichment step, as well as Illumina HiSeq, MiSeq and NexSeq, Life Technology Ion Torrent. Pacific Biosciences PacBio RSII, Oxford Nanopore MinIon, Promethlon and Gridlon and other massively parallel Multiplexed Sequencing Platforms.

### Microbe Detection

In some embodiments, the methods described herein may be used for detecting microbes, such as a virus as described herein, in samples. Such detection may comprise providing a sample as described herein with reagents for detection, incubating the sample or set of samples under conditions sufficient to allow binding of the primers or probes to nucleic acid corresponding to one or more microbe-specific targets wherein a positive signal is generated; and detecting the positive signal, wherein detection of the detectable positive signal indicates the presence of one or more target molecules from a microbe, i.e., a virus, in the sample. The one or more target molecules may be any type of nucleic acid, including, but not limited to, mRNA, rRNA, tRNA, genomic DNA (coding or non-coding), or a combination of any of these, wherein the nucleic acid comprises a target nucleotide sequence that may be used to distinguish two or more microbial species/strains from one another.

The embodiments disclosed herein may also utilize certain steps to improve hybridization and/or amplification between primers and/or probes of the invention and target nucleic acid sequences. Methods for enhancing nucleic acid hybridization and/or amplification are well-known in the art. A viral- or microbe-specific target may be a nucleic acid such as RNA or DNA, or a target may be a protein, such as a viral- or microbe-encoded protein.

In some embodiments, hybridization between a primer and/or probe of the invention and a viral or microbial target sequence may be performed to verify the presence of the virus and/or microbe in the sample. In some specific cases, one or more viruses or microbes may be detected simultaneously. In other embodiments, a primer and/or probe of the invention may distinguish between 2 or more different viruses or microbes, even where those viruses and/or microbes may be sufficiently similar at the nucleotide level.

### Detection of Single Nucleotide Variants

In some embodiments, one or more identified target sequences may be detected and/or differentiated using primers and/or probes of the invention that are specific for and bind to the target sequence as described herein. The systems and methods of the present invention can distinguish even between single nucleotide polymorphisms present among different viral or microbial species and therefore, use of multiple primers or probes in accordance with the invention may further expand on or improve the number of target sequences that may be used to distinguish between species. For example, in some embodiments, one or more primers and/or probes may distinguish between viruses and/or microbes at the species, genus, family, order, class, phylum, kingdom, or phenotype, or a combination thereof.

In certain example embodiments, a method or diagnostic test may be designed to screen viruses and/or microbes across multiple phylogenetic and/or phenotypic levels at the same time. For example, the method or diagnostic may comprise the use of multiple sets of primers and/or probes as described herein. Such an approach may be helpful for distinguishing viruses and/or microbes at the genus level, while further sets of primers/probes may distinguish at the species level. Thus, in accordance with the invention, a matrix may be produced identifying all viruses and/or microbes identified in a given sample. The foregoing is for example purposes only. Other means for classifying other microbe types are also contemplated and fall within the scope of the present invention so long as they find use of the primers and/or probes as described herein.

In certain other example embodiments, amplification of genetic material using a primer developed and/or described herein may be performed. Genetic material may comprise, for example, DNA and/or RNA, or a hybrid thereof, may be used to amplify the target nucleic acids. Amplification reactions employ recombinases, which are capable of pairing sequence-specific primers, such as described herein, with homologous sequence in the target nucleic acid, e.g., duplex DNA. If target DNA is present, DNA amplification is initiated and primers of the invention may anneal to the target sequence such that amplification of the target sequence may occur. Amplification reactions may be carried out at any appropriate temperature and using any reagents appropriate for the particular application or for the particular viral or microbial species. A primer of the invention is designed to amplify a sequence comprising the target nucleic acid sequence to be detected. In certain example embodiments, an RNA polymerase promoter, such as a T7 promoter, may be added to one of the primers, to result in an amplified double-stranded DNA product comprising the target sequence and an RNA polymerase promoter. After, or during, the amplification reaction, an RNA polymerase may be added that will produce RNA from the double-stranded DNA template. The amplified target RNA can then be detected as described herein. In this way, target DNA may be detected using the embodiments disclosed herein. Amplification reactions may also be used to amplify target RNA. The target RNA is first converted to cDNA using a reverse transcriptase reaction, followed by second strand DNA synthesis, at which point the amplification reaction proceeds as outlined above.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl2), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH4)2SO4], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or apatamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In certain embodiments, detection of DNA with the methods or systems of the invention requires transcription of the (amplified) DNA into RNA prior to detection.

### Set Cover Approaches

In particular embodiments, a primer and/or probe is designed that can identify, for example, all viral and/or microbial species within a defined set of viruses and microbes. Such methods are described in certain example embodiments. A set cover solution may identify the minimal number of target sequence probes or primers needed to cover an entire target sequence or set of target sequences, e.g. a set of genomic sequences. Set cover approaches have been used previously to identify primers and/or microarray probes, typically in the 20 to 50 base pair range. See, e.g. Pearson et al., cs.virginia.edu/-robins/papers/primers_dam11_final.pdf., Jabado et al. Nucleic Acids Res. 2006 34(22):6605-11, Jabado et al. Nucleic Acids Res. 2008, 36(1):e3 doi10.1093/nar/gkm1106, Duitama et al. Nucleic Acids Res. 2009, 37(8):2483-2492, Phillippy et al. BMC Bioinformatics. 2009, 10:293 doi:10.1186/1471-2105-10-293. Such approaches generally involved treating each primer/probe as k-mers and searching for exact matches or allowing for inexact matches using suffix arrays. In addition, the methods generally take a binary approach to detecting hybridization by selecting primers or probes such that each input sequence only needs to be bound by one primer or probe and the position of this binding along the sequence is irrelevant. Alternative methods may divide a target genome into pre-defined windows and effectively treat each window as a separate input sequence under the binary approach—i.e. they determine whether a given probe or guide RNA binds within each window and require that all of the windows be bound by the state of some primer or probe. Effectively, these approaches treat each element of the “universe” in the set cover problem as being either an entire input sequence or a pre-defined window of an input sequence, and each element is considered “covered” if the start of a probe or guide RNA binds within the element.

In some embodiments, the methods disclosed herein may be used to identify all variants of a given virus, or multiple different viruses in a single assay. Further, the method disclosed herein treat each element of the “universe” in the set cover problem as being a nucleotide of a target sequence, and each element is considered “covered” as long as a probe or guide RNA binds to some segment of a target genome that includes the element. Rather than only asking if a given primer or probe does or does not bind to a given window, such approaches may be used to detect a hybridization pattern—i.e. where a given primer or probe binds to a target sequence or target sequences—and then determines from those hybridization patterns the minimum number of primers or probes needed to cover the set of target sequences to a degree sufficient to enable both enrichment from a sample and sequencing of any and all target sequences. These hybridization patterns may be determined by defining certain parameters that minimize a loss function, thereby enabling identification of minimal probe or guide RNA sets in a way that allows parameters to vary for each species, e.g. to reflect the diversity of each species, as well as in a computationally efficient manner that cannot be achieved using a straightforward application of a set cover solution, such as those previously applied in the primer or probe design context.

The ability to detect multiple transcript abundances may allow for the generation of unique viral or microbial signatures indicative of a particular phenotype. Various machine learning techniques may be used to derive the gene signatures. Accordingly, the primers and/or probes of the invention may be used to identify and/or quantitate relative levels of biomarkers defined by the gene signature in order to detect certain phenotypes. In certain example embodiments, the gene signature indicates susceptibility to a particular treatment, resistance to a treatment, or a combination thereof.

In one aspect of the invention, a method comprises detecting one or more pathogens. In this manner, differentiation between infection of a subject by individual microbes may be obtained. In some embodiments, such differentiation may enable detection or diagnosis by a clinician of specific diseases, for example, different variants of a disease. Preferably the viral or pathogen sequence is a genome of the virus or pathogen or a fragment thereof. The method further may comprise determining the evolution of the pathogen. Determining the evolution of the pathogen may comprise identification of pathogen mutations, e.g. nucleotide deletion, nucleotide insertion, nucleotide substitution. Among the latter, there are non-synonymous, synonymous, and noncoding substitutions. Mutations are more frequently non-synonymous during an outbreak. The method may further comprise determining the substitution rate between two pathogen sequences analyzed as described above. Whether the mutations are deleterious or even adaptive would require functional analysis, however, the rate of non-synonymous mutations suggests that continued progression of this epidemic could afford an opportunity for pathogen adaptation, underscoring the need for rapid containment. Thus, the method may further comprise assessing the risk of viral adaptation, wherein the number non-synonymous mutations is determined. (Gire, et al., Science 345, 1369, 2014).

### Screening Environmental Samples

The methods disclosed herein may also be used to screen environmental samples for contaminants by detecting the presence of target nucleic acids or polypeptides. For example, in some embodiments, the invention provides a method of detecting viruses and/or microbes, comprising: exposing a primer and/or probe as described herein to a sample; allowing binding of the primer and/or probe to one or more viral- or microbe-specific target nucleic acids such that a detectable positive signal is produced. The positive signal can be detected and is indicative of the presence of one or more viruses or microbes in the sample.

As described herein, an environmental sample for use with the invention may be a biological or environmental sample, such as a food sample (fresh fruits or vegetables, meats), a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for the presence of viruses and/or microbes. Soil samples may be tested for the presence of pathogenic viruses or bacteria or other microbes, both for environmental purposes and/or for human, animal, or plant disease testing. Water samples such as freshwater samples, wastewater samples, or saline water samples can be evaluated for cleanliness and safety, and/or potability, to detect the presence of a viral or microbial contaminant such as, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, cerebrospinal fluid, ascites, pleural effusion, seroma, pus, or swab of skin or a mucosal membrane surface, or any other types of samples described herein above. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

A microbe in accordance with the invention may be a pathogenic virus or microbe or a microbe that results in food or consumable product spoilage. A pathogenic microbe may be pathogenic or otherwise undesirable to humans, animals, or plants. For human or animal purposes, a microbe may cause a disease or result in illness. Animal or veterinary applications of the present invention may identify animals infected with a microbe. For example, the methods and systems of the invention may identify companion animals with pathogens including, but not limited to, kennel cough, rabies virus, and heartworms. In other embodiments, the methods and systems of the invention may be used for parentage testing for breeding purposes. A plant microbe may result in harm or disease to a plant, reduction in yield, or alter traits such as color, taste, consistency, odor, For food or consumable contamination purposes, a microbe may adversely affect the taste, odor, color, consistency or other commercial properties of the food or consumable product. In certain example embodiments, the microbe is a bacterial species. The bacteria may be a psychrotroph, a coliform, a lactic acid bacteria, or a spore-forming bacteria. In certain example embodiments, the bacteria may be any bacterial species that causes disease or illness, or otherwise results in an unwanted product or trait. Bacteria in accordance with the invention may be pathogenic to humans, animals, or plants.

The invention is further described in the following examples, which do not limit the scope of the invention described in the claims.

### EXAMPLES

### Example 1—Genome Sequencing Reveals Zika Virus Diversity and Spread in the Americas

Despite great attention given to the recent Zika virus (ZIKV) epidemic in the Americas, much remains unknown about its epidemiology and evolution. One hundred ZIKV genomes were sequenced from clinical samples from 10 countries and territories, greatly expanding the observed viral genetic diversity from this outbreak, and analysis of the timing and patterns of introduction into distinct geographic regions was done. Phylogenetic evidence was confirmed for the origin and rapid expansion of the outbreak in Brazil (Faria et al., 2016), and for multiple introductions from Brazil into Honduras, Colombia, Puerto Rico, other Caribbean islands, and the continental US. It was found that ZIKV circulated undetected in many regions of the Americas for up to a year before the first reported diagnoses, highlighting the challenge of effective surveillance for this virus. Multiple sequencing approaches were developed and applied, optimizing genomic surveillance of ZIKV and characterizing genetic variation across the outbreak to identify mutations with possible functional implications for ZIKV biology and pathogenesis.

Since its introduction into the Americas in 2013 (Faria et al., 2016), mosquito-borne ZIKV (Family: Flaviviridae) has spread rapidly throughout the Americas, causing hundreds of thousands of cases of ZIKV disease, as well as ZIKV congenital syndrome and likely other neurological complications (Zika situation report, 2016; Dos Santos et al., 2016). Phylogenetic analysis of ZIKV can reveal the trajectory of the outbreak and detect mutations that may be associated with new disease phenotypes or affect molecular diagnostics. Despite the nearly 60 years since its discovery, however, fewer than 100 ZIKV genomes have been sequenced directly from clinical samples. This is due in part to technical challenges posed by low peak viral loads (for example, often orders of magnitude lower than in Ebola virus or dengue virus infection (Schieffelin et al., 2014; Sardi et al., 2016; Martina et al., 2009)), and practical challenges of sample handling because patient samples are typically collected for clinical diagnosis without sequencing in mind. Culturing the virus increases the material available for sequencing, but can result in genetic variation that is not representative of the original clinical sample.

In order to gain a deeper understanding of the viral populations underpinning the ZIKV epidemic, extensive genome sequencing was performed of ZIKV directly from samples collected as part of ongoing surveillance. Unbiased metagenomic RNA sequencing was initially pursued in order to capture both ZIKV and other viruses known to be co-circulating with ZIKV. In most of the 38 samples examined by this approach, there proved to be insufficient ZIKV RNA for genome assembly, but it still proved valuable to verify results from other methods. Metagenomic data also revealed RNA from other viruses, including 41 likely novel viral sequence fragments in mosquito pools (Table 1). In one patient, no ZIKV sequence was detected, but a complete genome from dengue virus was assembled (type 1), one of the viruses that co-circulates with and presents similarly to ZIKV.

TABLE 1
Viruses Identified from Metagenomic Data
a

| | | # reads | |
|---|---|---|---|
| | | from species | % genome |
| Species | Sample | (% of total) | unambiguous |
| Cell fusing | USA_2016_FL-01-MOS | 5662 | 99.1% |
| agent virus | | (0.02%) | |
| | USA_2016_FL-04-MOS | 1588 | 91.1% |
| | | (0.003%) | |
| | USA_2016_FL-05-MOS | 9614 | 99.9% |
| | | (0.02%) | |
| | USA_2016_FL-06-MOS | 2646 | 82.2% |
| | | (0.007%) | |
| | USA_2016_FL-08-MOS | 13608 | 99.4% |
| | | (0.008%) | |
| Deformed wing | USA_2016_FL-06-MOS | 6580 | 8.34% |
| virus-like | | (0.02%) | |
| Dengue | BLM_2016_MA-WGS16-006-SER | 2355926 | 99.8% |
| virus type 1 | | (2.6% | |
| JC polyomavirus | BRA_2016_FC-DQ75D1-URI | 8050 | 99.2% |
| | | (0.20%) | |
| JC polyomavirus- | USA_2016_FL-032-URI | 316 | 7.71% |
| like | | (0.001%) | |

b

| | | Classified | Classified | |
|---|---|---|---|---|
| | Total | contigs | contigs | Likely novel |
| Sample | contigs | (all) | (viral) | viral contigs |
| USA_2016_FL-01-MOS | 496 | 431 | 45 | 25 |
| USA_2016_FL-02-MOS | 563 | 463 | 17 | 14 |
| USA_2016_FL-03-MOS | 164 | 133 | 29 | 22 |
| USA_2016_FL-04-MOS | 679 | 492 | 25 | 19 |
| USA_2016_FL-05-MOS | 355 | 313 | 25 | 8 |
| USA_2016_FL-06-MOS | 726 | 635 | 26 | 14 |
| USA_2016_FL-07-MOS | 5967 | 5650 | 5 | 2 |
| USA_2016_FL-08-MOS | 1679 | 1528 | 39 | 27 |
| All pools: unique | 9013 | 8426 | 84 | 41 |

Viruses Other than Zika Uncovered by Unbiased Sequencing.
(a) Viral species other than Zika were found by unbiased sequencing of 38 samples. Column 3: number of reads in a sample belonging to a species as a raw count and a percent of total reads. Column 4: percent genome assembled based on the number of unambiguous bases called. Flavivirus cell fusing agent virus and deformed wing virus-like genomes in mosquito pools, and dengue virus type 1, JC polyomavirus, and JC polyomavirus-like genomes were identified in clinical samples. All assemblies had ≥95% sequence identity to a reference sequence for the listed species, except cell fusing agent virus in USA_2016_FL-06-MOS (91%) and dengue virus type 1 in BLM_2016_MA-WGS16-006-SER (92%). The dengue virus type 1 genome showed ≥95% sequence identity to other available isolates of the virus. (b) Contigs assembled from unbiased sequencing data of 8 mosquito pools. Column 2: number of contigs assembled. Column 3: number of contigs classified by BLASTNBLASTX43. Column 4: number of contigs hitting a viral species. Column 5: number of contigs hitting a viral species with <80% amino acid identity to the best hit. Each column is a subset of the previous column. Contigs in column 5 are considered to be likely novel. Last row lists counts, after removing duplicate contigs, for all mosquito pools combined.

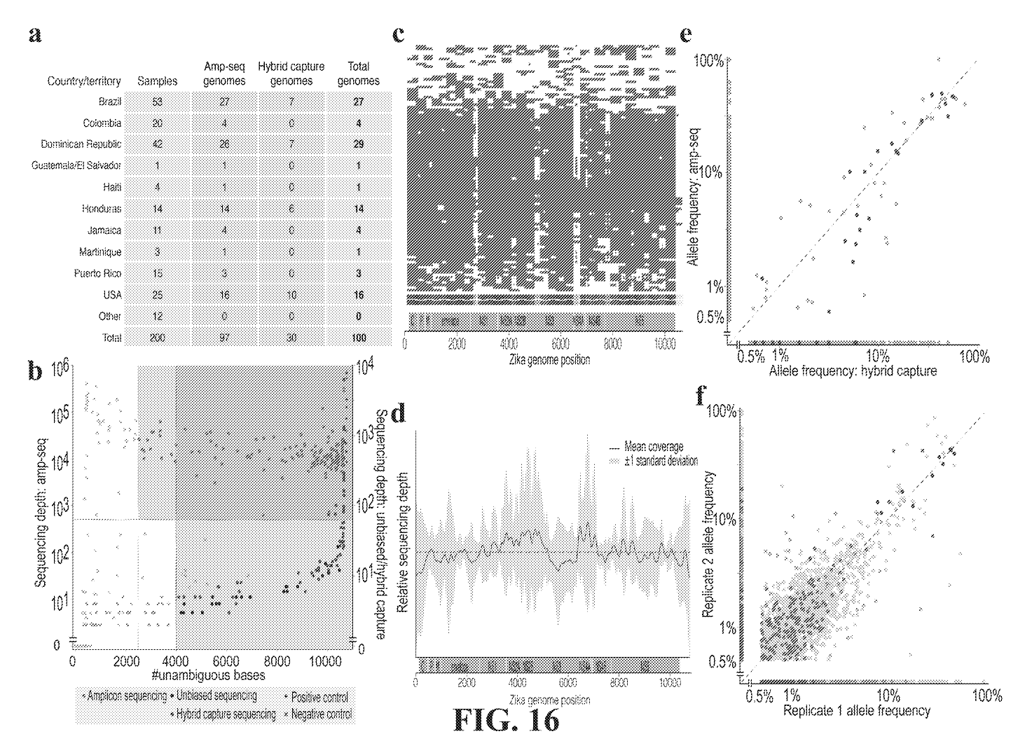

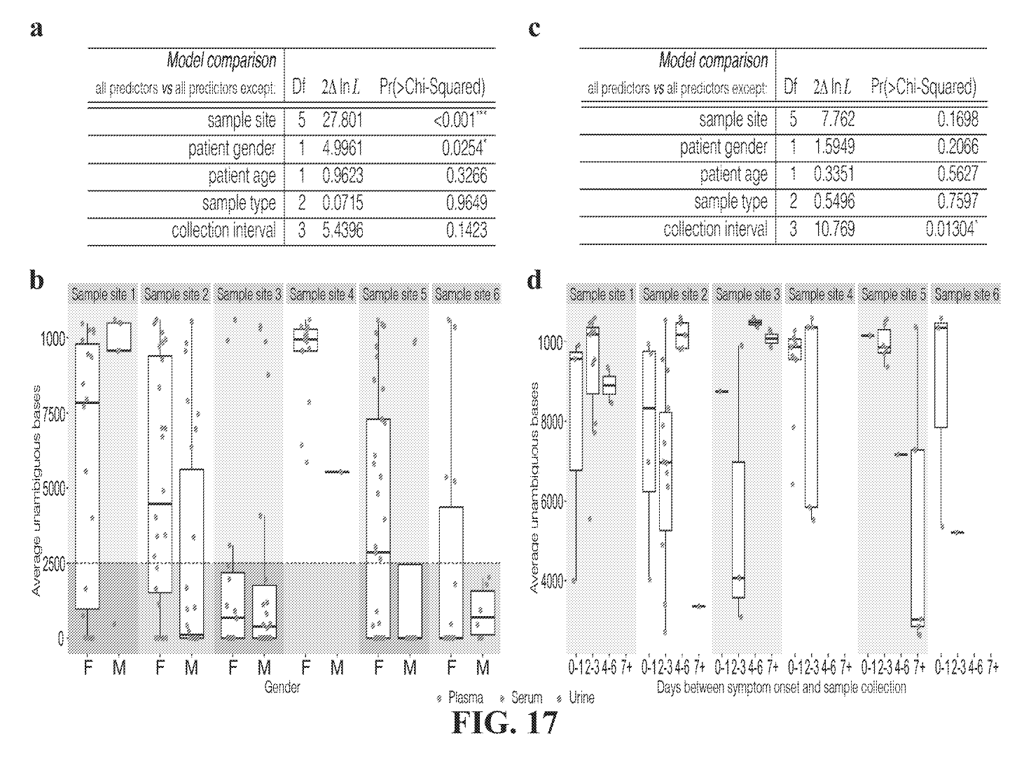

In order to capture sufficient ZIKV content for genome assembly, two targeted enrichment approaches were used before sequencing: multiplex PCR amplification and hybrid capture. Sequencing and assembly of complete or partial genomes from 110 samples from across the epidemic, out of 229 attempted (221 clinical samples from confirmed and possible ZIKV disease cases and eight mosquito pools, Table 4). This dataset, which was used for further analysis, included 110 genomes produced using multiplex PCR amplification (amplicon sequencing) and a subset of 37 genomes produced using hybrid capture (out of 66 attempted). Because these approaches amplify any contaminant ZIKV content, negative controls were relied heavily upon in order to detect artefactual sequence, and stringent, method-specific thresholds on coverage and completeness were established for calling high confidence ZIKV assemblies (FIG. 16a). Completeness and coverage for these genomes are shown in FIGS. 16b and 16c; the median fraction of the genome with unambiguous base calls was 93%. Per-base discordance between genomes produced by the two methods was 0.017% across the genome, 0.15% at polymorphic positions, and 2.2% for minor allele base calls. Concordance of within-sample variants is shown in more detail in FIG. 16d-16f. Patient sample type (urine, serum, or plasma) made no significant difference in sequencing success in the study (FIG. 17).

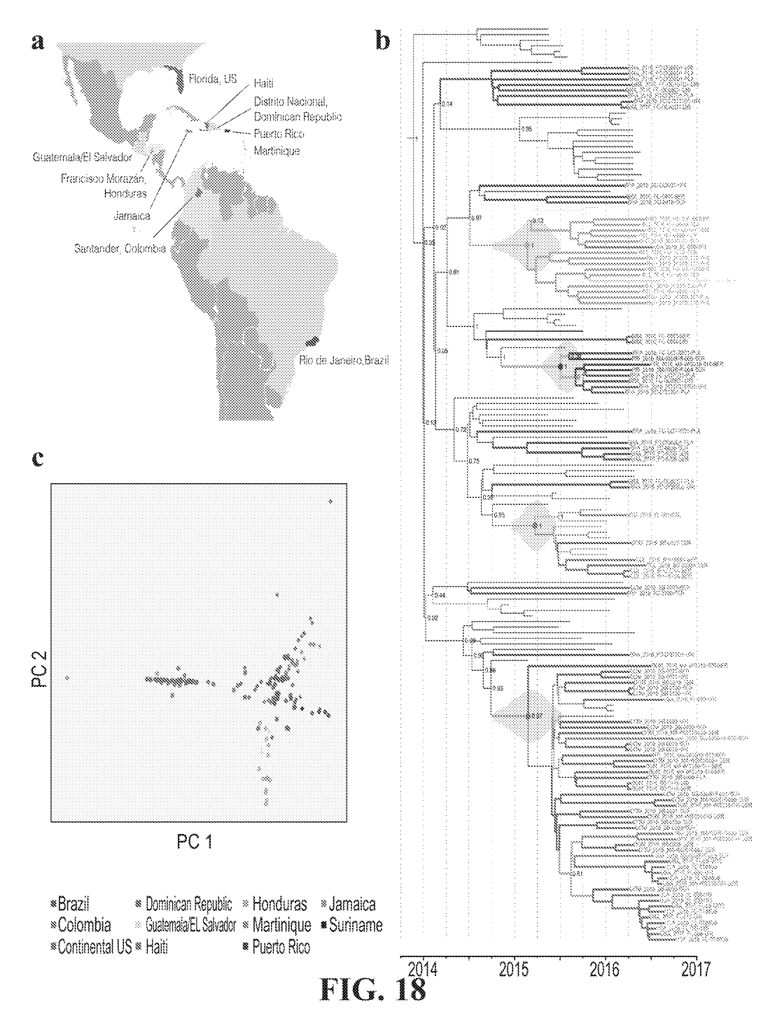

To investigate the spread of ZIKV in the Americas (FIG. 18), a phylogenetic analysis of the 110 genomes from the dataset was performed, together with 64 published genomes available on NCBI GenBank and in the literature (FIG. 18a). The reconstructed phylogeny (FIG. 18b), which is based on a molecular clock, is consistent with the outbreak originating in Brazil: Brazil ZIKV genomes appear on all deep branches of the tree, and their most recent common ancestor is the root of the entire tree. It was estimated that the date of that common ancestor to have been in early 2014 (95% credible interval, CI, August 2013 to July 2014). The shape of the tree near the root remains uncertain (i.e., the nodes have low posterior probabilities) because there are too few mutations to clearly distinguish the branches. This pattern suggests rapid early spread of the outbreak, consistent with the introduction of a new virus to an immunologically naïve population. ZIKV genomes from Colombia (n=10), Honduras (n=18), and Puerto Rico (n=3) cluster within distinct, well-supported clades. A clade consisting entirely of genomes from patients who contracted ZIKV in one of three Caribbean countries (the Dominican Republic, Jamaica, and Haiti) or the continental US, containing 30 of 32 genomes from the Dominican Republic and 19 of 20 from the continental US was also observed. The within-outbreak substitution rate was estimated to be 1.15×10⁻³ substitutions/site/year [95% CI (9.78×10⁻⁴, 1.33×10⁻³)], similar to prior estimates for this outbreak. This is somewhat higher (1.3×-5×) than reported rates for other flaviviruses¹³, but is measured over a short sampling period, and therefore may include a higher proportion of mildly deleterious mutations that have not yet been removed through purifying selection.

Determining when ZIKV arrived in specific regions helps elucidate the spread of the outbreak and track rising incidence of possible complications of ZIKV infection. The majority of the ZIKV genomes from the study fall into four major clades from different geographic regions, for which it was estimated a likely date for ZIKV arrival. In each case, the date was months earlier than the first confirmed, locally transmitted case, indicating ongoing local circulation of ZIKV before its detection. In Puerto Rico, the estimated date was 4.5 months earlier than the first confirmed local case¹⁴; it was 8 months earlier in Honduras¹⁵, 5.5 months earlier in Colombia¹⁶, and 9 months earlier for the Caribbean/continental US clade¹⁷. In each case, the arrival date represents the estimated time to the most recent common ancestor (tMRCA) for the corresponding clade in our phylogeny (FIG. 18c). Similar temporal gaps between the tMRCA of local transmission chains and the earliest detected cases were seen when chikungunya virus emerged in the Americas. Evidence for several introductions of ZIKV into the continental US was observed, and it was found that sequences from mosquito and human samples collected in Florida cluster together, consistent with the finding of local ZIKV transmission in Florida.

Principal component analysis (PCA) is consistent with the phylogenetic observations (FIG. 17d). It shows tight clustering among ZIKV genomes from the continental US, the Dominican Republic, and Jamaica. ZIKV genomes from Brazil and Colombia are similar and distinct from genomes sampled in other countries. ZIKV genomes from Honduras form a third cluster that also contains genomes from Guatemala or El Salvador. The PCA results show no clear stratification of ZIKV within Brazil.

Determining when ZIKV arrived in specific regions is important for understanding the epidemiology of the virus and its effects on health. The tMRCA was estimated for well-supported nodes within the phylogeny, including four highly supported clades (posterior probability >0.95), formed mostly by strains from Colombia, Honduras, Puerto Rico, and the Caribbean. It was found that these four clades originated in early to mid 2015, many months before ZIKV was first reported in each region, indicating ongoing local circulation of ZIKV before its detection by surveillance systems. The tMRCA of Colombian sequences was estimated to be in March 2015 [95% CI (2014.97, 2015.46)], 7 months before the first confirmed cases in Colombia (Pacheco et al., (2016), Zika virus disease in Colombia-preliminary report. New England Journal of Medicine); Honduran sequences to be in March 2015 [95% CI (2014.76, 2015.50)], 10 months before the first reported case (Pan-American Health Organization. Zika-Epidemiological Report Honduras, paho.org/hq/index.php?option=com_docman&task=doc_view&gid=35137&Itemid=270), and Puerto Rican sequences to be in July 2015 [95% CI (2015.30, 2015.78)], six months before the first reported case (Pan-American Health Organization. Zika-Epidemiological Report Puerto Rico, paho.org/hq/index.php?option=com_docman&task=doc_view&gid=35231&Itemid=270 &lang-en). The estimated tMRCA of the Caribbean clade, consisting of sequences from three Caribbean countries and the continental USA, to be in February 2015 [95% CI (2014.76, 2015.52)], seven months before the first reported case in the Dominican Republic and about nine months before the first reported case in Florida, USA (Likos et al., “Local Mosquito-Borne Transmission of Zika Virus—Miami-Dade and Broward Counties, Fla., June-August 2016,” MMWR Morb Mortal Wkly Rep 65:1032-1038, 2016). Several introductions of ZIKV into the continental USA were observed and it was found that sequences from mosquito and human samples collected in Florida cluster together, consistent with previous findings. Similar temporal gaps between the tMRCA of local transmission chains and the detection of early cases were observed in the emergence of chikungunya in the Americas (Nunes et al., 2015).

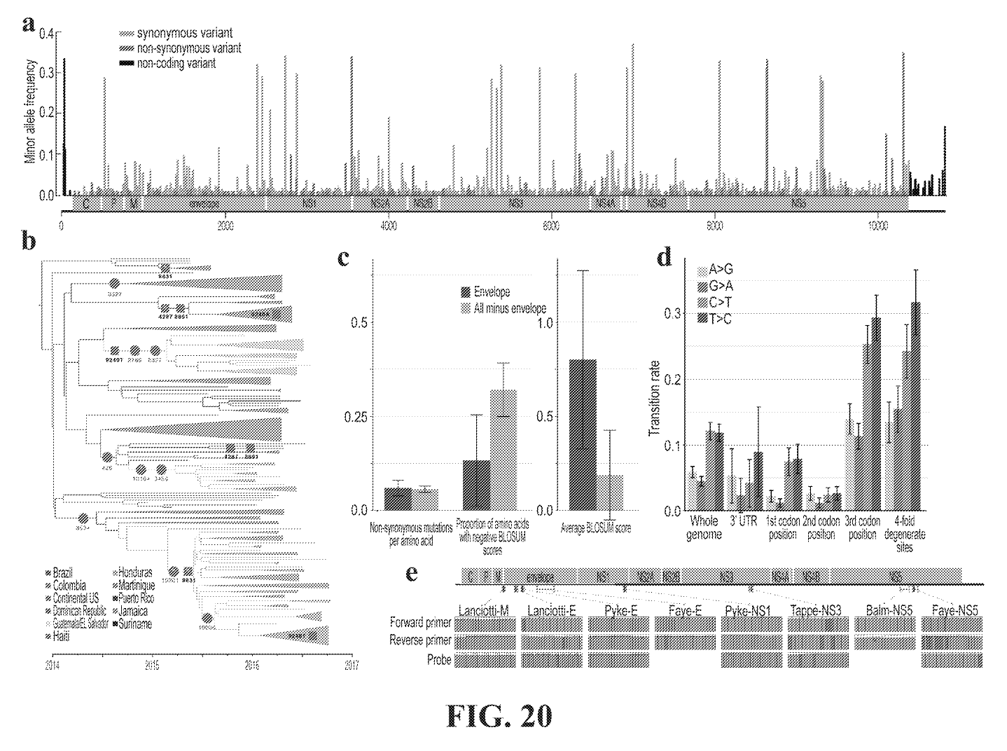

Genetic variation can provide important clues to understanding ZIKV biology and pathogenesis and can reveal potentially functional changes in the virus. 1030 single nucleotide polymorphisms (SNPs) were observed in the complete dataset, well distributed across the genome (FIG. 20a). Any effect of these mutations cannot be determined from these data; however, the most likely candidates for functional mutations would be among the 202 nonsynonymous SNPs (Table 5) and the 32 SNPs in the 5′ and 3′ untranslated regions (UTRs). Adaptive mutations are more likely to be found at high frequency or to be seen multiple times, although both effects can also occur by chance. Five positions with nonsynonymous mutations were observed at >5% minor allele frequency that occur on two or more branches of the tree (FIG. 20b); two of these (at 4287 and 8991) occur together and might represent incorrect placement of a Brazil branch in the tree. The remaining three are more likely to represent multiple nonsynonymous mutations; one (at 9240) appears to involve nonsynonymous mutations to two different alleles.

To assess the possible biological significance of these mutations, evidence of selection in the ZIKV genome was evaluated. Viral surface glycoproteins are known targets of positive selection, and mutations in these proteins can confer adaptation to new vectors¹⁹ or aid immune escape^{20,21}. An excess of nonsynonymous mutations was evaluated in the ZIKV envelope glycoprotein (E). However, the nonsynonymous substitution rate in E proved to be similar to that in the rest of the coding region (FIG. 20c, left); moreover, amino acid changes were significantly more conservative in that region than elsewhere (FIG. 20c, middle and right). Any diversifying selection occurring in the surface protein thus appears to be operating under selective constraint. Evidence was also identified for purifying selection in the ZIKV 3′ UTR (FIG. 20d, Table 6), a region important for viral replication²².

While the transition-to-transversion ratio (6.98) in the dataset was within the range seen in other viruses (Duchene et al., 2015), a significantly higher frequency of C-to-T and T-to-C substitutions than other transitions was observed (FIG. 20d and Table 2). This enrichment was apparent both in the genome as a whole and at 4-fold degenerate sites, where selection pressure is minimal. Many processes are possible contributors to this conspicuous mutation pattern, including mutational bias of the ZIKV RNA-dependent RNA polymerase, host RNA editing enzymes (e.g., APOBECs, ADARs) acting upon viral RNA, and chemical deamination, but further investigation is required to determine the actual cause of this phenomenon.

TABLE 2
Nucleotide transition and transversion rates. Observed nucleotide
changes in 165 outbreak genomes, per available base.

| | | | |
|---|---|---|---|
| to A | to C | to G | to T |

Extended Data Table 2a | All mutations
per available base.

| | | | | |
|---|---|---|---|---|
| from A | 0.00000 | 0.00439 | 0.05914 | 0.00912 |
| from C | 0.00880 | 0.00000 | 0.00084 | 0.12154 |
| from G | 0.04502 | 0.00349 | 0.00000 | 0.00476 |
| from T | 0.01083 | 0.11867 | 0.00520 | 0.00000 |

Extended Data Table 2b | 3′ UTR mutations,
per available base.

| | | | | |
|---|---|---|---|---|
| from A | 0.00000 | 0.00000 | 0.05310 | 0.00885 |
| from C | 0.00000 | 0.00000 | 0.00000 | 0.04202 |
| from G | 0.02326 | 0.00000 | 0.00000 | 0.00000 |
| from T | 0.00000 | 0.08955 | 0.00000 | 0.00000 |

Extended Data Table 2c | Mutations, fourfold
degenerate sites, per available base.

| | | | | |
|---|---|---|---|---|
| from A | 0.00000 | 0.01263 | 0.13474 | 0.03579 |
| from C | 0.02079 | 0.00000 | 0.00000 | 0.24249 |
| from G | 0.15461 | 0.00998 | 0.00000 | 0.01746 |
| from T | 0.03779 | 0.31686 | 0.02326 | 0.00000 |

Extended Data Table 2d | Codon position 1 mutations,
per available base.

| | | | | |
|---|---|---|---|---|
| from A | 0.00000 | 0.00478 | 0.02199 | 0.00287 |
| from C | 0.00678 | 0.00000 | 0.00000 | 0.07458 |
| from G | 0.01219 | 0.00000 | 0.00000 | 0.00325 |
| from T | 0.01257 | 0.07899 | 0.00359 | 0.00000 |

Extended Data Table 2e | Codon position 2 mutations,
per available base.

| | | | | |
|---|---|---|---|---|
| from A | 0.00000 | 0.00000 | 0.02649 | 0.00331 |
| from C | 0.00255 | 0.00000 | 0.00128 | 0.02423 |
| from G | 0.01186 | 0.00527 | 0.00000 | 0.00132 |
| from T | 0.00103 | 0.02667 | 0.00000 | 0.00000 |

Extended Data Table 2f | position 3 mutations,
per available base.

| | | | | |
|---|---|---|---|---|
| from A | 0.00000 | 0.00694 | 0.13988 | 0.02312 |
| from C | 0.01595 | 0.00000 | 0.00000 | 0.25285 |
| from G | 0.11332 | 0.00497 | 0.00000 | 0.00895 |
| from T | 0.02370 | 0.29333 | 0.01333 | 0.00000 |

Mismatches between PCR assays and viral sequence are a potential source of poor diagnostic performance in this outbreak²⁴. To assess the potential impact of ongoing viral evolution on diagnostic function, we compared eight published qRT-PCR-based primer/probe sets to our data. Numerous sites were found where the probe or primer did not match an allele found among the 174 ZIKV genomes from the current dataset (FIG. 20e). In most cases, the discordant allele was shared by all outbreak samples, presumably because it was present in the Asian lineage that entered the Americas. These mismatches could affect all uses of the diagnostic assay in the outbreak. Mismatches were found from new mutations that occurred following ZIKV entry into the Americas. Most of these were present in less than 10% of samples, although one was seen in 29%. These observations suggest that genome evolution has not caused widespread degradation of diagnostic performance during the course of the outbreak, but that mutations continue to accumulate and ongoing monitoring is needed.

Analysis of within-host viral genetic diversity can reveal important information for understanding virus-host interactions and viral transmission. However, accurately identifying these variants in low-titer clinical samples is challenging, and further complicated by potential artefacts associated with enrichment prior to sequencing. To investigate whether it was possible to reliably detect within-host ZIKV variants in the data, within-host variants were identified in a cultured ZIKV isolate used as a positive control throughout the study, and it was found that both amplicon sequencing and hybrid capture data produced concordant and replicable variant calls (FIG. 16d). In clinical samples, hybrid capture within-host variants were noisier but contained a reliable subset: although most variants were not validated by the other sequencing method or by a technical replicate, those at high frequency were always replicable, as were those that passed a previously described filter²⁵ (FIG. 16e-f, Table 3). Within this high confidence set, variants shared between samples were evaluated as a clue to transmission patterns, but there were too few variants to draw any meaningful conclusions. By contrast, within-host variants identified in amplicon sequencing data were unreliable at all frequencies (FIG. 16f, Table 3), suggesting that further technical development is needed before amplicon sequencing can be used to study within-host variation in ZIKV and other clinical samples with low viral titer.

TABLE 3
Unvalidated Variants Across Methods.
a

| | % unvalidated |
|---|---|
| Method | by other method |
| Amplicon sequencing | 87.3% n = 128 |
| Hybrid capture | 85.8% n = 113 |
| Hybrid capture, verified | 25.0% n = 20 |

b

% unvalidated in replicate

| | all | variants passing |
|---|---|---|
| Method | variants | strand bias filter |
| Amplicon sequencing | 92.7% n = 304 | 66.7% n = 3 |
| Hybrid capture | 74.5% n = 98 | 0.00% n = 8 |

Sequencing low titer viruses like ZIKV directly from clinical samples presents several challenges that have likely contributed to the paucity of genomes available from the current outbreak. While development of technical and analytical methods will surely continue, it is noted that factors upstream in the process, including collection site and cohort, were strong predictors of sequencing success in the study (FIG. 17). This highlights the importance of continuing development and implementation of best practices for sample handling, without disrupting standard clinical workflows, for wider adoption of genome surveillance during outbreaks. Additional sequencing, however challenging, remains critical to ongoing investigation of ZIKV biology and pathogenesis. Together with two companion studies^{10,11}, this effort advances both technological and collaborative strategies for genome surveillance in the face of unexpected outbreak challenges.

### Methods

### Sample Collections and Study Subjects

Human blood, urine, cerebrospinal fluid, and saliva samples were obtained from suspected ZIKV cases; all samples were acquired during the period in which the participant was symptomatic. A blood sample of up to 5 mL was taken from the patient/research subject via venipuncture using sterile and disposable material, similar to blood collections during routine laboratory tests. The time from onset of symptoms to enrollment into respective studies was similar among different patients. Following sample acquisition, specimens were stored between 4 and −20′C. Serum or plasma were prepared by centrifugation at 2,500 rpm for 15 min using whole blood or anticoagulated blood, respectively. Diagnostic tests for the presence of ZIKV were performed on-site using RT-qPCR or RT-PCR (see below).

### Viral RNA Isolation

RNA was isolated following manufacturer's standard operating protocol for 0.14 mL up to 1 mL samples³² using the QIAamp Viral RNA Minikit (Qiagen), except that in some cases 0.1 M final concentration of β-mercaptoethanol (as a reducing agent) or 40 μg/mL final concentration of linear acrylamide (Ambion) (as a carrier) were added to AVL buffer prior to inactivation. Extracted RNA was resuspended in AVE buffer or nuclease-free water. In some cases, viral samples were concentrated using Vivaspin-500 centrifugal concentrators (Sigma-Aldrich) prior to inactivation and extraction. In these cases, 0.84 mL of sample was concentrated to 0.14 mL by passing through a 30 kDa filter and discarding the flow through.

### Quantification of RNA Content Using RT-qPCR

Host RNA (18S rRNA) was quantified using the Power SYBR Green RNA-to-Ct 1-Step kit (Applied Biosystems) and human 18S rRNA primers: 5′-TCCTTTAACGAGGATCCATTGG-3′ (forward, SEQ ID NO:1), and 5′-CGAGCTTTTTAACTGCAGCAACT-3′ (reverse, SEQ ID NO:2). Human genomic DNA (Promega) was used as a standard control. All reactions were performed on the ABI 7900HT (Applied Biosystems). ZIKV samples were quantified using a panel of published RT-qPCR assays which included two assays that target the envelope (E) region as described by (Pyke et al., 2014) and (Lanciotti et al. 2008) and one assay that targets the nonstructural protein 5 (NS5) gene as described by (Faye et al., 2013). Standards for each assay were created using IDT gBlocks® Gene Fragments. Standard curves for each assay were created by performing a 10-fold serial dilution of all assay standards resulting in a dynamic range of 1×10⁷to 1 copies/pl. All RT-qPCR assays were performed in 10 μl reactions using TaqMan RNA-to-CT 1-Step Kit (Applied Biosystems) and 3 μl of a 1:20 dilution of sample RNA or standard. Genome amplification was performed on the ABI 7900HT and QuantStudio™ 6 Real Flex Real-Time PCR System (ThermoFisher Scientific) using the conditions previously described for each assay (Pyke et al., 2014; Lanciotti et al., 2008; Faye et al., 2013).

Carrier RNA and Host rRNA Depletion

In a subset of samples, carrier RNA and host rRNA were depleted from RNA samples using RNase H selective depletion (Morlan et al., 2012; Matranga et al., 2014). Briefly, oligo d(T) (40 nt long) and/or DNA probes complementary to human rRNA were hybridized to the sample RNA. The sample was then treated with 20 units of Hybridase Thermostable RNase H (Epicentre) for 30 minutes at 45° C. The complementary DNA probes were removed by treating each reaction with RNase-free DNase kit (Qiagen) according to the manufacturer's protocol. Depleted samples were purified using 2.2× volume AMPure RNAclean beads (Beckman Coulter Genomics) and eluted into 10 μl water for cDNA synthesis.

### Illumina Library Construction and Sequencing

cDNA synthesis was performed as described in previously published RNA-seq methods⁹. To track potential cross-contamination, 50 fg of synthetic RNA (gift from M. Salit, NIST) was spiked into samples using unique RNA for each individual ZIKV sample. ZIKV negative control cDNA libraries were prepared from water, human K-562 total RNA (Ambion), or EBOV (KY425633.1) seed stock; ZIKV positive controls were prepared from ZIKV Senegal (isolate HD78788) or ZIKV Pernambuco (isolate PE243; KX197192.1) seed stock. The dual index Accel-NGS® 2S Plus DNA Library Kit (Swift Biosciences) was used for library preparation. Approximately half of the cDNA product was used for library construction, and indexed libraries were generated using 18 cycles of PCR. Each individual sample was indexed with a unique barcode. Libraries were pooled at equal molarity and sequenced on the Illumina HiSeq 2500 or MiSeq (paired-end reads) platforms.

Amplicon-Based cDNA Synthesis and Library Construction

ZIKV amplicons were prepared as described^{8,11}, similarly to “RNA jackhammering” for preparing low input viral samples for sequencing³⁴, with slight modifications. After PCR amplification, each amplicon pool was quantified on a 2200 Tapestation (Agilent Technologies) using High Sensitivity D1000 ScreenTape (Agilent Technologies). 2 μL of a 1:10 dilution of the amplicon cDNA was loaded and the concentration of the 350-550 bp fragments was calculated. The cDNA concentration, as reported by the Tapestation, was highly predictive of sequencing outcome (i.e., whether a sample passes genome assembly thresholds). cDNA from each of the two amplicon pools were mixed equally (10-25 ng each) and libraries were prepared using the dual index Accel-NGS@ 2S Plus DNA Library Kit (Swift Biosciences) according to manufacturer's protocol. Libraries were indexed with a unique barcode using 7 cycles of PCR, pooled equally, and sequenced on the Illumina MiSeq (250 bp paired-end reads) platform. Primer sequences were removed by hard trimming the first 30 bases for each insert read prior to analysis.

### Zika Hybrid Capture

Viral hybrid capture was done as previously described (Matranga et al., 2014). Probes were created to target ZIKV and Chikungunya virus (CHIKV). Candidate probes were created by tiling across publicly available sequences for ZIKV and CHIKV (NCBI GenBank). Probes were selected from among these candidate probes to minimize the number used while maintaining coverage of the observed diversity of the viruses. Alternating universal adapters were added to allow two separate PCR amplifications, each consisting of non-overlapping probes.

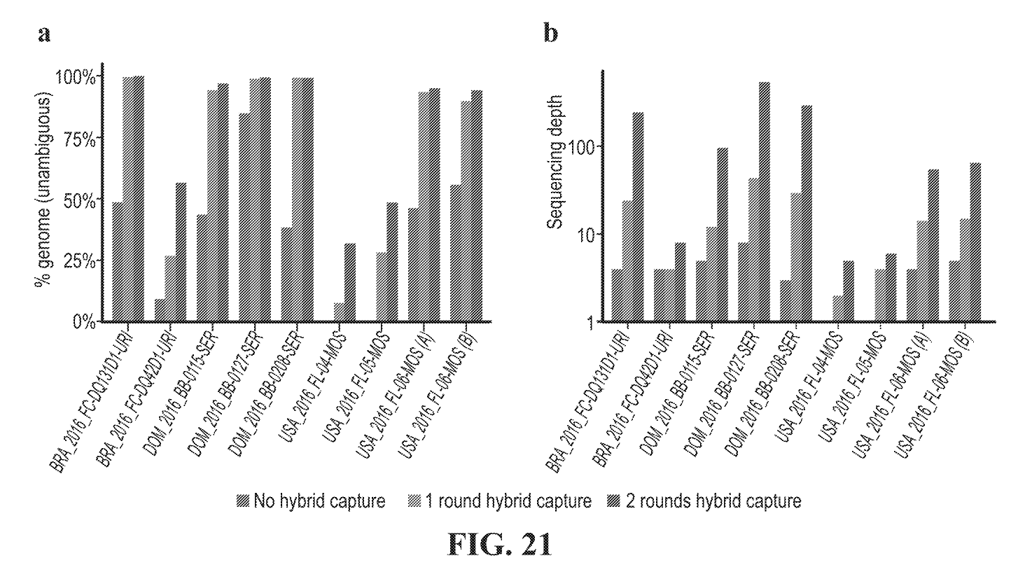

The probes were synthesized on a 12k array (CustomArray). The synthesized oligos were amplified by two separate emulsion PCR reactions with primers containing T7 RNA polymerase promoter. Biotinylated baits were in vitro transcribed (MEGAshortscript, Ambion) and added to prepared ZIKV libraries. The baits and libraries were hybridized overnight (˜16 hrs), captured on streptavidin beads, washed, and re-amplified by PCR using the Illumina adapter sequences. Capture libraries were then pooled and sequenced. In some cases, a second round of hybrid capture was performed on PCR-amplified capture libraries to further enrich the ZIKV content of sequencing libraries (FIG. 21). In the main text, “hybrid capture” refers to a combination of hybrid capture sequencing data and data from the same libraries without capture (unbiased), unless explicitly distinguished.

### Genome Assembly

Reads were assembled from all sequencing methods into genomes using viral-ngs v1.13.3^{36,37}. Reads were filtered taxonomically from amplicon sequencing against a ZIKV reference, KU321639.1. Reads were filtered from other approaches against the list of accessions provided herein. To compute results on individual replicates, we de novo assembled these and scaffolded against KU321639.1. To obtain final genomes for analysis, data was pooled from multiple replicates of a sample, de novo assembled, and scaffolded against KX197192.1. For all assemblies, the viral-ngs ‘assembly_min_length_fraction_of_reference’ and ‘assembly_min_unambig’ parameters were set to 0.01. For amplicon sequencing data, unambiguous base calls required at least 90% of reads to agree in order to call that allele (‘major_cutoff’=0.9); for hybrid capture data, the default threshold of 50% was used. Viral-ngs were modified so that calls to GATK's UnifiedGenotyper set ‘min_indel_count_for_genotyping’ to 2.

At 3 sites with insertions or deletions (indels) in the consensus genome CDS, the genome was corrected using Sanger sequencing of the RT-PCR product (namely, at 3447 in the genome for sample DOM_2016_BB-0085-SER; at 5469 in BRA_2016_FC-DQ12D1-PLA; and at 6516-6564 in BRA_2016_FC-DQ107D1-URI, with coordinates in KX197192.1). At other indels in the consensus genome CDS, indels with ambiguity were replaced.

When reporting and using depth of coverage values from amplicon-based sequencing data, PCR and optical duplicates were not removed. Otherwise, these were removed with viral-ngs.

### Identification of Viruses in Samples by Unbiased Sequencing

Using kraken v0.10.6 (Wood et al., 2014) in viral-ngs, a database was built that includes its default “full” database (which incorporates all bacterial and viral whole genomes from RefSeq (O'Leary et al., 2016) as of October 2015). Additionally included were the whole human genome (hg38), genomes from PlasmoDB (Aurrecoechea et al., 2009), and sequences covering mosquito genomes (Aedes aegypti, Aedes albopictus, Anopheles albimanus, Anopheles quadrimaculatus, Culex quinquefasciatus, and the outgroup Drosophila melanogaster) from GenBank (Clark et al., 2016), protozoa and fungi whole genomes from RefSeq, SILVA LTP 16s rRNA sequences (Yarza et al., 2008), and all sequences from NCBI's viral accession list (as of October 2015) for viral taxa that have human as a host.

For each sample, Kraken was run and its output reports were searched for viral taxa with more than 100 reported reads. The results were manually filtered to remove ZIKV, bacteriophages, and likely lab contaminants. For each sample and its associated taxa, genomes were assembled using viral-ngs as described above. The following genomes were used for taxonomically filtering reads and as the reference for assembly: KJ741267.1 (cell fusing agent virus), AY292384.1 (deformed wing virus), and LC164349.1 (JC polyomavirus). When reporting sequence identity of an assembly with a taxon, the identity used was that determined by BLASTN (Altschul et al., 1997) when the assembly compared against the reference genome used for assembly.

To focus on metagenomics of mosquito pools (Table 1), unbiased sequencing data from 8 mosquito pools were considered (not including hybrid capture data). First the depletion pipeline of viral-ngs was run on raw data and then run on the viral-ngs Trinity⁴⁴ assembly pipeline on the depleted reads to assemble them into contigs. Contigs from all mosquito pool samples were pooled and all duplicate contigs were identified with sequence identity >95% using CD-HIT⁴⁵. Additionally, predicted coding sequences from Prodigal 2.6.3⁴⁶ were used to identify duplicate protein sequences at >95% identity. Contigs were classified using BLASTN⁴³ against nt and BLASTX⁴³ against nr (as of February 2017) and contigs with an e-value greater than 1E-4 were discarded. Viral contigs are defined as contigs that hit a viral sequence, and all reverse-transcriptase-like contigs were removed due to their similarity to retrotransposon elements within the Aedes aegypti genome. Viral contigs with less than 80% amino acid identity to their best hit as likely novel viral contig were categorized. Table 9 lists the unique viral contigs found, their best hit, and information scoring the hit.

### Relationship Between Metadata and Sequencing Outcome

To determine if metadata are predictive of sequencing outcome, the following variables were tested: sample collection site, patient gender, patient age, sample type, and the number of days between symptom onset and sample collection (“collection interval”). To describe sequencing outcome of a sample S, the following response variable Y_{S} were used: mean({I(R)*(number of unambiguous bases in R) for all amp-seq replicates R of S}), where I(R)=1 if median depth of coverage of R≥500 and I(R)=0 otherwise.

The one sample of type “Saliva,” the one sample of type “Cerebrospinal fluid,” the samples from mosquito pools, and rows with missing values were excluded. Samples with type “Plasma EDTA” were treated as having type “Plasma,” and the “collection interval” variable was treated as categorical (0-1, 2-3, 4-6, and 7+ days).

With a single model, the zero counts were underfit, possibly because many zeros (no positive Zika virus assembly) are truly Zika-negative. The data is thus viewed as coming from two processes: one determining whether a sample is Zika-positive or Zika-negative, and another that determines, among the observed positive samples, how much of a Zika genome that is able to be sequenced. The first process was modeled with logistic regression (in R using GLM (R Core Team 2016) with binomial family and logit link); the positive observed samples are the samples S for which Y_{S}≥2500. For the second, a beta regression was performed, using only the positive observed samples, of Y_{S} divided by Zika genome length on the predictor variables. This was implemented in R using the betareg package (Cribari-Neto et al., 2010) and fractions from the closed unit interval were transformed to the open unit interval as the authors suggest.

To test the significance of predictor variables, a likelihood ratio test was used. For variable Xᵢ, a full model (with all predictors) was compared against a model that uses all predictors except Xᵢ. Results are shown in FIG. 17.

### Visualization of Coverage Depth Across Genomes

For amplicon-based sequencing data, coverage was plotted across 97 samples that yielded a positive assembly by either method and for which amplicon-based data was obtained (FIG. 16c). With viral-ngs, depleted reads were aligned to the reference sequence KX197192.1 using the novoalign aligner with options ‘-r Random -1 40 -g 40 -×20 -t 100 -k’. There was no duplicate removal. Depth was binarized at each nucleotide position, showing red if depth of coverage was at least 500×. Rows (samples) were hierarchically clustered to ease visualization.

For hybrid capture sequencing data, depth of coverage was plotted across the 37 samples that yielded a passing assembly (FIG. 16c). Reads were aligned as described above for amplicon sequencing data, except duplicates were removed. For each sample, depth of coverage was calculated at each nucleotide position. The values for each sample were then scaled so that each would have a mean depth of 1.0. At each nucleotide position, the median depth across the samples was calculated, as well as the 20^{th} and 80^{th} percentiles. The mean of each of these metrics was plotted within a 200-nt sliding window.

### Criteria for Pooling Across Replicates

Sequencing was attempted for one or more replicates of each sample and a genome assembled from each replicate. Data from any replicates whose assembly showed high sequence similarity was discarded, in any part of the genome, to the assembly of a sample consisting of an African (Senegal) lineage (strain HD78788). This sample was used as a positive control throughout this study, and its presence was considered in the assembly of a clinical sample to be evidence of contamination. Any data from replicates that showed evidence of contamination was also discarded, at the RNA stage, by the baits used for hybrid capture; these were detected by looking for adapters that were added to these probes for amplification.

For the amplicon sequencing approach, an assembly was considered positive if it contained at least 2500 unambiguous base calls and had a median depth of coverage of at least 500× over its unambiguous bases (depth was calculated including duplicate reads). For the unbiased and hybrid capture approaches, an assembly of a replicate was considered positive if it contained at least 4000 unambiguous base calls at any coverage depth. For each approach, the unambiguous base threshold was selected based on an observed density of negative controls below the threshold (FIG. 16b). For assemblies from amplicon sequencing data, a threshold on depth of coverage was added because coverage depth was roughly binary across replicates, with negative controls falling in the lower class. Based on these thresholds, it was found that 0 of 87 negative controls used throughout the sequencing runs yielded positive assemblies and that 29 of 29 positive controls yielded positive assemblies.

A sample was considered to have a positive assembly if any of its replicates, by either method, yielded an assembly that passed the above thresholds. For each sample with at least one positive assembly, read data was pooled across replicates for each sample, including replicates with assemblies that did not pass the positivity thresholds. When data was available by both amplicon-based sequencing and unbiased/hybrid capture approaches, amplicon sequencing data was pooled separately from data produced by the unbiased and hybrid capture approaches, the latter two of which were pooled together (henceforth, the “hybrid capture” pool). A genome was then assembled from each set of pooled data. When assemblies on pooled data were available from both approaches, the assembly was selected from the hybrid capture approach if it had more than 10267 unambiguous base calls (95% of the reference, GenBank accession KX197192.1); when both assemblies had fewer than this number of unambiguous base calls, the one that had more unambiguous base calls was selected.

The number of ZIKV genomes publicly available prior to this study was the result of a GenBank (Clark et al., 2016) search for ZIKV in February 2017. Any sequences with length <4000 nt were filtered, and sequences that were part of the present study or that were labeled as having been passaged were excluded. Less than 100 sequences were counted.

### Multiple Sequence Alignments

ZIKV consensus genomes were aligned using MAFFT v7.221 (Katoh et al., 2013) with the following parameters: ‘-maxiterate 1000-ep 0.123-localpair’.

Analysis of within- and Between-Sample Variants

To measure overall per-base discordance between consensus genomes produced by amp-seq and hybrid capture, all sites where base calls were made in both the amp-seq and hybrid capture consensus genomes of a sample were considered, and the fraction in which the alleles were not in agreement was calculated. To measure discordance at minor alleles, all of the consensus genomes generated in this study that were selected for downstream analysis were searched for minor alleles (see Criteria for pooling across replicates for choosing among the amp-seq and hybrid capture genome when both are available). All positions at which there was a minor allele and for which genomes from both methods were available were evaluated, and the fraction in which the alleles were not in agreement were calculated. For both calculations, partial ambiguity was tolerated (e.g., ‘Y’ is concordant with ‘T’). If one genome had full ambiguity (‘N’) at a position and the other genome had an indel, the site was counted as discordant; otherwise, if one genome had full ambiguity, it was not counted.

After assembling genomes, within-sample allele frequencies were determined for each sample by running V-Phaser 2.0 via viral-ngs³⁷ on all pooled reads mapping to the sample assembly. When determining per-library allele counts at each variant position, viral-ngs were modified to require a minimum base (Phred) quality score of 30 for all bases, to discard anomalous read pairs, and to use per-base alignment quality (BAQ) in its calls to SAMtools⁵⁰ mpileup. This was particularly helpful for filtering spurious amplicon sequencing variants because all generated reads start and end at a limited number of positions (due to the pre-determined tiling of amplicons across the genome). Because amplicon sequencing libraries were sequenced using 250 bp paired-end reads, bases near the middle of the ˜450 nt amplicons fall at the end of both paired reads, where quality scores drop and incorrect base calls are more likely. To determine the overall frequency of each variant in a sample, allele counts were summed (calculated using SAMtools⁵⁰ mpileup via viral-ngs) across libraries.

When comparing allele frequencies across methods: let fₐ and f_{hc} be frequencies in amplicon sequencing and hybrid capture, respectively. If both were non-zero, an allele was included only if the read depth at its position was ≥1/min(fₐ, f_{hc}) in both methods, and if depth at the position was at least 100 for hybrid capture and 275 for amplicon sequencing. If fₐ=0, a read depth of max(1/f_{hc}, 275) at the position in the amplicon sequencing method was used; similarly, if f_{hc}=0 a read depth of max(1/fₐ, 100) at the position in the hybrid capture method was used. This was to eliminate lack of coverage as a reason for discrepancy between two methods. When comparing allele frequencies across sequencing replicates within a method, only a minimum read depth (275× for amplicon sequencing and 100× for hybrid capture) was imposed, but required this depth in both libraries. In samples with more than two replicates, only the two replicates with the highest depth at each plotted position were considered.

Allele frequencies from hybrid capture sequencing were considered to be “verified” if they passed the strand bias and frequency filters described in Gire et al., 2014, with the exception that a variant identified in only one library was allowed if its frequency was ≥5%. In Table 8 and FIG. 16f, the same strand bias filter was applied, but not the minimum frequency filter. In FIGS. 16e and f, alleles were considered “validated” if they were present at above 0.5% frequency in both libraries or methods. When comparing two libraries for a given method M(amp-seq or hybrid capture): the proportion unvalidated was the fraction, among all variants in M at ≥0.5% frequency in at least one library, of the variants that are at ≥0.5% frequency in exactly one of the two libraries. Similarly, when comparing methods: the proportion unvalidated for a method M was the fraction, among all variants at ≥20.5% frequency in M, of the variants that are at ≥20.5% frequency in M and <0.5% frequency in the other method. The root mean squared error includes only points found in both methods or replicates (i.e., does not include unvalidated alleles). Restricting the sample set used for comparison of alleles across libraries to only samples with a positive assembly in both methods had no significant impact on the results.

SNPs were initially called on the aligned consensus genomes using Geneious version 9.1.7 (Kearse et al., 2012). Since Geneious treats ambiguous base calls as variants, the SNP set was filtered and allele frequencies were re-calculated directly from the consensus genomes, treating fully or partially ambiguous calls as missing data. A nonsynonymous SNP is shown on the tree (FIG. 20b) if it includes an allele that is nonsynonymous relative to the ancestral state (see Molecular clock phylogenetics and ancestral state reconstruction section below) and has an allele frequency of >5%; all occurrences of nonsynonymous alleles are shown. Mutations were placed at a node such that the node leads only to samples with the mutation or with no call at that site. Uncertainty in placement occurs when a sample lacks a base call for the corresponding SNP; in this case, the SNP was placed on the most recent branch for which data was available. This ancestral ZIKV state was used to count the frequency of each type of substitution over various regions of the ZIKV genome, per number of available bases in each region (FIG. 20d and Table 8).

The effect of nonsynonymous SNPs was quantified using the original BLOSUM62 scoring matrix for amino acids (Henikoff and Henikoff 1992), in which positive scores indicate conservative amino acid changes and negative scores unlikely or extreme substitutions. Statistical significance was assessed for equality of proportions by χ² test (FIG. 20c, middle), and for difference of means by 2-sample t-test with Welch-Satterthwaite approximation of df (FIG. 20c, right). All error bars indicate 95% confidence intervals.

### Maximum Likelihood Estimation and Root-to-Tip Regression

A maximum likelihood tree was generated using a multiple sequence alignment that includes sequences generated in this study, as well as a selection of other available sequences from the Americas, Southeast Asia, and Pacific. IQ-TREE (Nguyen et al., 2015) was run with options ‘-m HKY+G4 -bb 1000’ (Minh et al., 2013). In FigTree v1.4.2 (Rambaut 2014), the tree was rooted on the oldest sequence used as input (GenBank accession EU545988. 1).

TempEst v1.5 was used (Rambaut et al., 2016), which selects the best-fitting root with a residual mean squared function (also EU545988.1), to estimate root-to-tip distances. Regression was performed in R with the 1m function (R Core Team 2016) of distances on dates.

### Molecular Clock Phylogenetics and Ancestral State Reconstruction

For molecular clock phylogenetics, a multiple sequence alignment was made from the genomes generated in this study combined with a selection of other available sequences from the Americas. Sequences from outside the outbreak in the Americas were not used. Among ZIKV genomes published and publicly available on NCBI GenBank³⁵, 32 were selected from the Americas that had at least 7000 unambiguous bases, were not labeled as having been passaged more than once, and had location metadata. In addition, 32 genomes from Brazil published in a companion paper¹⁰ that met the same criteria were used.

BEAST v1.8.4 was used to perform molecular clock analyses⁵⁶. Sampled tip dates were used to handle inexact dates⁵⁷. Because of sparse data in non-coding regions, only the CDS was used as input. The SDR06 substitution model was used on the CDS, which uses HKY with gamma site heterogeneity and partitions codons into two partitions (positions (1+2) and 3)⁵⁸. To perform model selection, three coalescent tree priors were tested: a constant-size population, an exponential growth population, and a Bayesian Skyline tree prior (10 groups, piecewise-constant model)⁵⁹. For each tree prior, two clock models were tested: a strict clock and an uncorrelated relaxed clock with lognormal distribution (UCLN)⁶⁰. In each case, the molecular clock rate was set to use a continuous time Markov chain rate reference prior⁶¹. For all six combinations of models, path sampling (PS) and stepping-stone sampling (SS) were performed to estimate marginal likelihood^{62,63}. Sampling was done for 100 path steps with a chain length of 1 million, with power posteriors determined from evenly spaced quantiles of a Beta(alpha=0.3; 1.0) distribution. The Skyline tree prior provided a better fit than the two other (baseline) tree priors (Table 7), so this tree was used prior for all further analyses. Using a constant or exponential tree prior, a relaxed clock provides a better model fit, as shown by the log Bayes factor when comparing the two clock models. Using a Skyline tree prior, the log Bayes factor comparing a strict and relaxed clock is smaller than it is using the other tree priors, and it is similar to the variability between estimated log marginal likelihood from PS and SS methods. A relaxed clock was chosen for further analyses, but key findings were also reported using a strict clock.

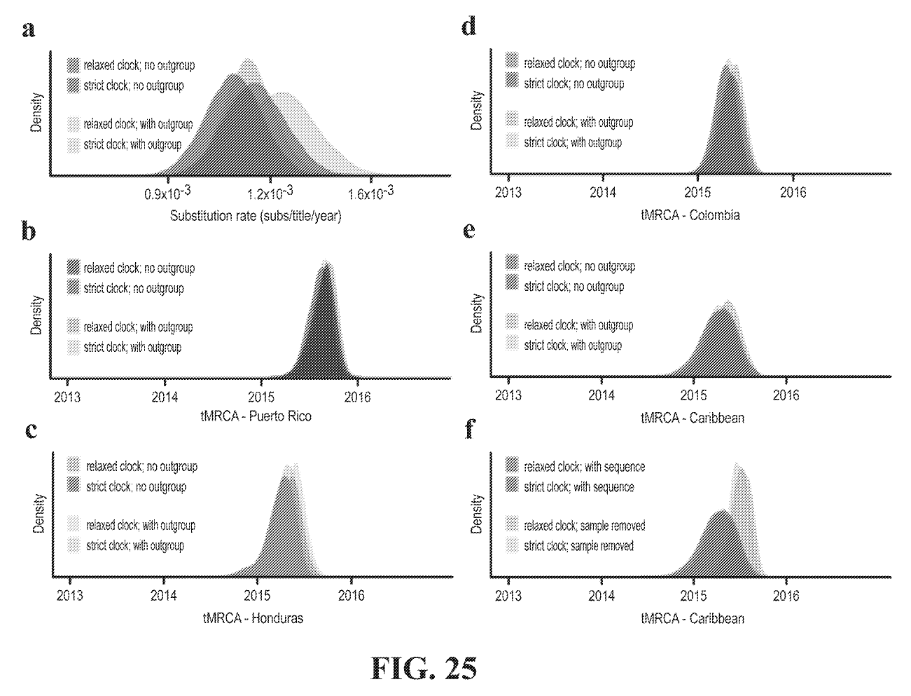

For the tree and tMRCA estimates in FIG. 17, as well as the clock rate reported in main text, BEAST was run with 400 million MCMC steps using the SRD06 substitution model, Skyline tree prior, and relaxed clock model. Clock rate and tMRCA estimates, and their distributions, were extracted with Tracer v1.6.0 and the maximum clade credibility (MCC) tree was identified using TreeAnnotator v1.8.2. The reported credible intervals around estimates are 95% highest posterior density (HPD) intervals. When reporting substitution rate from a relaxed clock model, the mean rate was given (mean of the rates of each branch weighted by the time length of the branch). Additionally, for the tMRCA estimates in FIG. 17c with a strict clock, BEAST was run with the same specifications (also with 400M steps) except used a strict clock model. The resulting data are also used in the more comprehensive comparison shown in FIG. 25.

For the data with an outgroup in FIG. 25, BEAST was run the same as specified above (with strict and relaxed clock models), except with 100 million steps and with outgroup sequences in the input alignment. The outgroup sequences were the same as those used to make the maximum likelihood tree. For the data excluding sample DOM_2016_MA-WGS 16-020-SER in FIG. 25, BEAST was run the same as specified above (with strict and relaxed clocks), except this sample was removed from the input and 100 million steps were run.

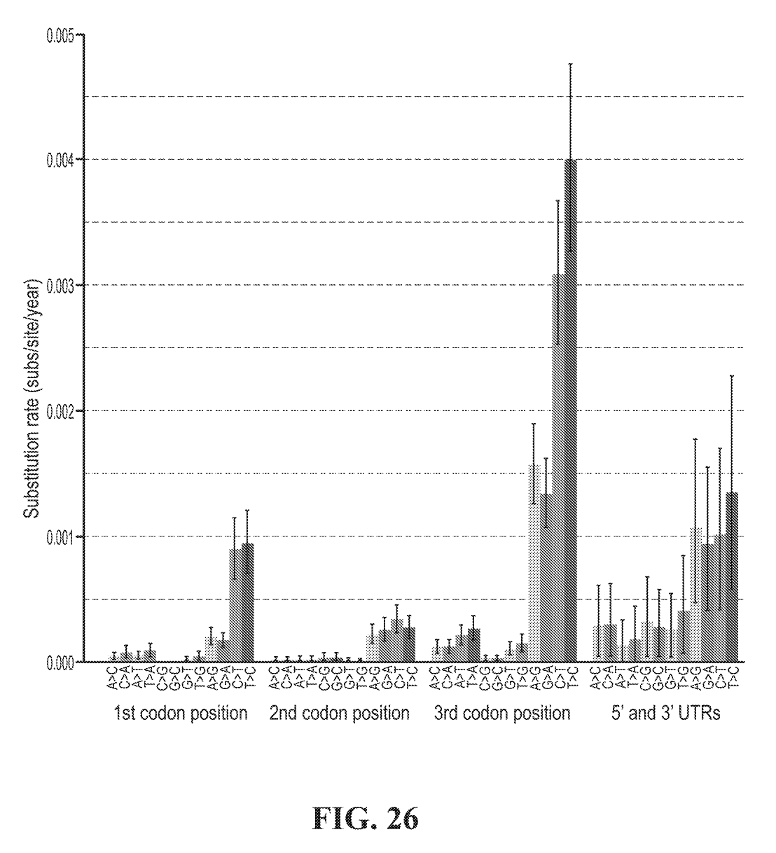

BEAST v1.8.4 was used to estimate transition and transversion rates with CDS and non-coding regions. The model was the same as above except that we used the Yang96 substitution model on the CDS, which uses GTR with gamma site heterogeneity and partitions codons into three partitions⁶⁴; for the non-coding regions, a GTR substitution model was used with gamma site heterogeneity and no codon partitioning. There were four partitions in total: one for each codon position and another for the non-coding region (5′ and 3′ UTRs combined). This was run for 200 million steps. At each sampled step of the MCMC, substitution rates were calculated for each partition using the overall substitution rate, the relative substitution rate of the partition, the relative rates of substitutions in the partition, and base frequencies. In FIG. 26, the means of these rates over the steps were plotted; the error bars shown are 95% HPD intervals of the rates over the steps.

BEAST v1.8.4 was used to reconstruct ancestral state at the root of the tree using CDS and non-coding regions. The model was the same as above except that, on the CDS, the HKY substitution model was used with gamma site heterogeneity and codons partitioned into three partitions (one per codon position). On the non-coding regions the same substitution model was used without codon partitioning. This was run for 50 million steps and TreeAnnotator v1.8.2 was used to find the state with the MCC tree. The ancestral state was selected corresponding to this state. In all BEAST runs, the first 10% of states were discarded from each run as burn-in.

### Principal Components Analysis

PCA was conducted using the R package FactoMineR (Le et al., 2008). Missing data was imputed with the package missMDA (Josse et al., 2016). Removing the two most extreme outlier samples from the plot clarified population structure, and the results are shown in FIG. 18b.

### Diagnostic Assay Assessment

Primer and probe sequences (FIG. 20e) were extracted from eight published RT-qPCR assays (Pyke et al., 2014; Lanciotti et al., 2008; Faye et al., 2008, 2013; Balm et al., 2012; Tappe et al., 2014) and aligned to our ZIKV genomes using Geneious version 9.1.7. (Kearse et al., 2012). Matches and mismatches were then tabulated to the diagnostic sequence for all outbreak genomes, allowing multiple bases to match where the diagnostic primer and/or probe sequence contained nucleotide ambiguity codes. Sequences used in the present study are provided in Table 3.

Links to publicly available data used in methods Hybrid capture probes that target Zika and Chikungunya viruses storage.googleapis.com/sabeti-public/hybsel_probes/zikv-chikv_201602.fasta [2.25 MB]. Probe sequences are 140 nt. They contain 20 nt adapters on each end for PCR amplification; the middle 100 nt targets the virus.

Kraken database built for identifying viruses in samples by unbiased sequencing storage.googleapis.com/sabeti-public/meta_db s/kraken_full-and-mosquito-and-all_human_viral.tar.gz [185.25 GB]

Sequences used for taxonomic filtering or analyses Sequences against which reads from unbiased and hybrid capture approaches were taxonomically filtered.

GenBank accessions: KX087101.2 KX198135.1 KX101066.1 KU501215.1 KX197192.1 KU365779.1 KU991811.1 KU681082.3 KU955589.1 KU926309.1 KU321639.1 KX087102.1 KX253996.1 HQ234500.1 KF383115.1 KU955591.1 KF383117.1 KU955593.1 KF383119.1 KX156775.1 KU922923.1 KU729218.1 KF268950.1 KU820899.2 KU866423.1 NC_012532.1 KU365777.1 KU955590.1 KF268948.1 KU501216.1 KU647676.1 KX198134.1 KU963574.1 KU527068.1 KU937936.1 KX101062.1 KX262887.1 DQ859059.1 KX051563.1 KU820897.2 KU497555.1 KU926310.1 KU681081.3 KU707826.1 KU509998.3 AY632535.2 KX156774.1 KX247646.1 KU820898.1 KU365780.1 HQ234501.1 KU940228.1 HQ234498.1 KU955592.1 KF383118.1 JN860885.1 KU365778.1 KU955595.1 KX185891.1 KU922960.1 KX156776.1 KJ776791.1 KU853013.1 KU744693.1 KX056898.1 KF383116.1 KU761564.1 KU963796.1 KU853012.1 KU312312.1 LC002520.1 HQ234499.1 KU963573.1 KU729217.2 KU870645.1 KF993678.1 KU501217.1 KF383120.1 KF268949.1 KX117076.1 EU545988.1 KU955594.1

Sequences used in molecular clock phylogenetic analyses and SNP analyses All sequences generated in this study, as well as: ● 32 published sequences from the Americas. GenBank accessions: KU312312.1 KU321639.1 KU365777.1 KU365778.1 KU365779.1 KU497555.1 KU501216.1 KU501217.1 KU509998.3 KU527068.1 KU647676.1 KU707826.1 KU729217.2 KU729218.1 KU820897.5 KU853012.1 KU853013.1 KU926310.1 KU940224.1 KU940227.1 KU940228.1 KX051563.1 KX101060.1 KX101061.1 KX101066.1 KX269878.1 KX280026.1. 5 sequences from Colombia, with permission from the authors. GenBank accessions: KY317936.1 KY317937.1 KY317938.1 KY317939.1 KY317940.1. 32 sequences generated in the ZiBRA project, with permission from the authors. ZiBRA project IDs: ZBRA105 ZBRC14 ZBRC16 ZBRC18 ZBRC28 ZBRC301 ZBRC302 ZBRC313 ZBRC319 ZBRC321 ZBRD103 ZBRD107 ZBRD116 ZBRX1 ZBRX2 ZBRX4 ZBRX7 ZBRX8 ZBRX11 ZBRX12 ZBRX13 ZBRX14 ZBRX15 ZBRX16 ZBRX100 ZBRX102 ZBRX103 ZBRX106 ZBRX127 ZBRX128 ZBRX130 ZBRX137

Sequences used for maximum likelihood estimation and root-to-tip regression. Sequences from “Sequences used in molecular clock phylogenetic analyses and SNP analyses” as well as 6 outgroup sequences from Southeast Asia and the South Pacific. These outgroup sequences are: ● 6 published sequences. GenBank accessions: EU545988.1 JN860885.1 KF993678.1 KJ776791.2 KU681081.3 KU681082.3

Table 4 listed below provides observed non-synonymous SNPs across the data used for SNP analysis. Includes frequency and count of ancestral and derived alleles at each position, as well as amino acid changes caused by each SNP.

| | | | | | | | Impact on ZIKV proteins | | | | |
|---|---|---|---|---|---|---|---|---|---|---|---|
| Genome | | | | | | | | | | Amino | |
| Position (in | Allele | | Frequency within outbreak | | Number of Alleles | | Codon | Degen- | | acid | |
| KX197192.1) | Ancesteral | Derived | Ancesteral | Derived | Minor | Total | position | eracy | Codon change | change | Protein |
| 138 | T | C | 0.99306 | 0.00694 | 1 | 144 | 11 | 1 | TTC −> CTC | F −> L | capsid |
| 186 | T | C | 0.99301 | 0.00699 | 1 | 143 | 27 | 1 | TTT −> CTT | F −> L | capsid |
| 346 | T | C | 0.97222 | 0.02778 | 4 | 144 | 80 | 1 | ATA −> ACA | I −> T | capsid |
| 350 | A | G | 0.9932 | 0.0068 | 1 | 147 | 81 | 3 | ATA −> ATG | I −> M | capsid |
| 420 | G | A | 0.99375 | 0.00625 | 1 | 160 | 105 | 1 | GGC −> AGC | G −> S | capsid |
| 428 | T | A | 0.85535 | 0.13208 | 21 | 159 | 107 | 2 | GAT −> GAA | D −> E | capsid |
| 439 | G | A | 0.99375 | 0.00625 | 1 | 160 | 111 | 1 | GGA −> GAA | G −> E | capsid |
| 444 | G | A | 0.9875 | 0.0125 | 2 | 160 | 113 | 1 | GTT −> ATT | V −> I | capsid |
| 524 | C | A | 0.99333 | 0.00667 | 1 | 150 | 17 | 2 | AAC −> AAA | N −> K | propeptide |
| 604 | T | C | 0.99338 | 0.00662 | 1 | 151 | 44 | 1 | ATG −> ACG | M −> T | propeptide |
| 606 | T | G | 0.99338 | 0.00662 | 1 | 151 | 45 | 1 | TGT −> GGT | C −> G | propeptide |
| 616 | C | T | 0.99329 | 0.00671 | 1 | 149 | 48 | 1 | ACC −> ATC | T −> I | propeptide |
| 666 | G | A | 0.99315 | 0.00685 | 1 | 146 | 65 | 1 | GTC −> ATC | V −> I | propeptide |
| 709 | G | T | 0.99355 | 0.00645 | 1 | 155 | 79 | 1 | GGA −> GTA | G −> V | propeptide |
| 792 | A | G | 0.98675 | 0.01325 | 2 | 151 | 14 | 1 | ACG −> GCG | T −> A | membrane |
| 810 | T | A | 0.99254 | 0.00746 | 1 | 134 | 20 | 2 | TTG −> ATG | L −> M | membrane |
| 1044 | G | A | 0.97333 | 0.02667 | 4 | 150 | 23 | 1 | GTT −> ATT | V −> I | envelope |
| 1116 | A | T | 0.99296 | 0.00704 | 1 | 142 | 47 | 1 | ACA −> TCA | T −> S | envelope |
| 1143 | G | A | 0.98592 | 0.01408 | 2 | 142 | 56 | 2 | GTA −> ATA | V −> I | envelope |
| 1152 | T | C | 0.99296 | 0.00704 | 1 | 142 | 59 | 1 | TAC −> CAC | Y −> H | envelope |
| 1167 | T | A | 0.99301 | 0.00699 | 1 | 143 | 64 | 1 | TCA −> ACA | S −> T | envelope |
| 1180 | T | C | 0.99301 | 0.00699 | 1 | 143 | 68 | 1 | ATG −> ACG | M −> T | envelope |
| 1181 | G | A | 0.99301 | 0.00699 | 1 | 143 | 68 | 3 | ATG −> ATA | M −> I | envelope |
| 1183 | C | T | 0.99301 | 0.00699 | 1 | 143 | 69 | 1 | GCT −> GTT | A −> V | envelope |
| 1360 | A | G | 0.99315 | 0.00685 | 1 | 146 | 128 | 1 | AAG −> AGG | K −> R | envelope |
| 1387 | A | T | 0.99324 | 0.00676 | 1 | 148 | 137 | 1 | TAC −> TTC | Y −> F | envelope |
| 1521 | G | A | 0.99254 | 0.00746 | 1 | 134 | 182 | 1 | GGT −> AGT | G −> S | envelope |
| 1561 | G | C | 0.9863 | 0.0137 | 2 | 146 | 195 | 1 | GGC −> GCC | G −> A | envelope |
| 1714 | A | G | 0.99338 | 0.00662 | 1 | 151 | 246 | 1 | AAG −> AGG | K −> R | envelope |
| 1718 | C | A | 0.99329 | 0.00671 | 1 | 149 | 247 | 2 | GAC −> GAA | D −> E | envelope |
| 1741 | T | C | 0.99324 | 0.00676 | 1 | 148 | 255 | 1 | GTC −> GCC | V −> A | envelope |
| 1747 | T | C | 0.99333 | 0.00667 | 1 | 150 | 257 | 1 | GTT −> GCT | V −> A | envelope |
| 1756 | G | C | 0.99329 | 0.00671 | 1 | 149 | 260 | 1 | AGT −> ACT | S −> T | envelope |
| 1816 | C | T | 0.99324 | 0.00676 | 1 | 148 | 280 | 1 | GCA −> GTA | A −> V | envelope |
| 1902 | A | T | 0.99359 | 0.00641 | 1 | 156 | 309 | 1 | ACC −> TCC | T −> S | envelope |
| 1903 | C | A | 0.98077 | 0.01923 | 3 | 156 | 309 | 1 | ACC −> AAC | T −> N | envelope |
| 1965 | G | T | 0.99359 | 0.00641 | 1 | 156 | 330 | 2 | GTA −> TTA | V −> L | envelope |
| 1980 | A | G | 0.99333 | 0.00667 | 1 | 150 | 335 | 1 | ACA −> GCA | T −> A | envelope |
| 2023 | T | C | 0.99329 | 0.00671 | 1 | 149 | 349 | 1 | ATG −> ACG | M −> T | envelope |
| 2028 | A | T | 0.99329 | 0.00671 | 1 | 149 | 351 | 1 | ACT −> TCT | T −> S | envelope |
| 2079 | A | G | 0.99338 | 0.00662 | 1 | 151 | 368 | 1 | AGC −> GGC | S −> G | envelope |
| 2083 | C | T | 0.99333 | 0.00667 | 1 | 150 | 369 | 1 | ACT −> ATT | T −> I | envelope |
| 2169 | C | T | 0.99351 | 0.00649 | 1 | 154 | 398 | 1 | CAC −> TAC | H −> Y | envelope |
| 2319 | A | G | 0.99286 | 0.00714 | 1 | 140 | 448 | 1 | ATT −> GTT | I −> V | envelope |
| 2412 | A | G | 0.99296 | 0.00704 | 1 | 142 | 479 | 1 | ACA −> GCA | T −> A | envelope |
| 2449 | T | C | 0.99296 | 0.00704 | 1 | 142 | 491 | 2 | TTA −> TCA | L −> S | envelope |
| 2511 | T | C | 0.98684 | 0.01316 | 2 | 152 | 8 | 1 | TTC −> CTC | F −> L | NS1 |
| 2527 | C | T | 0.99342 | 0.00658 | 1 | 152 | 13 | 1 | ACG −> ATG | T −> M | NS1 |
| 2678 | G | A | 0.99291 | 0.00709 | 1 | 141 | 63 | 3 | ATG −> ATA | M −> I | NS1 |
| 2781 | T | C | 0.99355 | 0.00645 | 1 | 155 | 98 | 2 | TGG −> CGG | W −> R | NS1 |
| 2788 | G | C | 0.90323 | 0.09677 | 15 | 155 | 100 | 1 | GGT −> GCT | G −> A | NS1 |
| 2795 | G | T | 0.99355 | 0.00645 | 1 | 155 | 102 | 2 | CAG −> CAT | Q −> H | NS1 |
| 2853 | T | C | 0.96129 | 0.03871 | 6 | 155 | 122 | 1 | TAC −> CAC | Y −> H | NS1 |
| 2923 | T | C | 0.99306 | 0.00694 | 1 | 144 | 145 | 1 | CTC −> CCC | L −> P | NS1 |
| 2925 | A | G | 0.99306 | 0.00694 | 1 | 144 | 146 | 1 | AAA −> GAA | K −> E | NS1 |
| 2944 | G | A | 0.99315 | 0.00685 | 1 | 146 | 152 | 1 | AGC −> AAC | S −> N | NS1 |
| 2946 | T | C | 0.9863 | 0.0137 | 2 | 146 | 153 | 1 | TTT −> CTT | F −> L | NS1 |
| 3034 | C | T | 0.99306 | 0.00694 | 1 | 144 | 182 | 1 | GCC −> GTC | A −> V | NS1 |
| 3058 | G | A | 0.99338 | 0.00662 | 1 | 151 | 190 | 1 | GGA −> GAA | G −> E | NS1 |
| 3061 | A | G | 0.98026 | 0.01974 | 3 | 152 | 191 | 1 | AAG −> AGG | K −> R | NS1 |
| 3070 | T | C | 0.97368 | 0.02632 | 4 | 152 | 194 | 1 | GTA −> GCA | V −> A | NS1 |
| 3112 | A | G | 0.99346 | 0.00654 | 1 | 153 | 208 | 1 | GAC −> GGC | D −> G | NS1 |
| 3186 | A | G | 0.99291 | 0.00709 | 1 | 141 | 233 | 1 | ACA −> GCA | T −> A | NS1 |
| 3214 | T | C | 0.99291 | 0.00709 | 1 | 141 | 242 | 1 | ATC −> ACC | I −> T | NS1 |
| 3223 | A | G | 0.99286 | 0.00714 | 1 | 140 | 245 | 1 | AAG −> AGG | K −> R | NS1 |
| 3246 | C | T | 0.99286 | 0.00714 | 1 | 140 | 253 | 1 | CAT −> TAT | H −> Y | NS1 |
| 3315 | C | T | 0.99301 | 0.00699 | 1 | 143 | 276 | 2 | CGG −> TGG | R −> W | NS1 |
| 3459 | C | T | 0.92357 | 0.07643 | 12 | 157 | 324 | 2 | CGG −> TGG | R −> W | NS1 |
| 3514 | A | G | 0.99338 | 0.00662 | 1 | 151 | 342 | 1 | GAA −> GGA | E −> G | NS1 |
| 3534 | A | G | 0.66225 | 0.33775 | 51 | 151 | 349 | 2 | ATG −> GTG | M −> V | NS1 |
| 3535 | T | C | 0.99338 | 0.00662 | 1 | 151 | 349 | 1 | ATG −> ACG | M −> T | NS1 |
| 3601 | T | C | 0.99333 | 0.00667 | 1 | 150 | 19 | 1 | ATG −> ACG | M −> T | NS2A |
| 3631 | C | T | 0.99329 | 0.00671 | 1 | 149 | 29 | 1 | ACC −> ATC | T −> I | NS2A |
| 3658 | T | C | 0.99329 | 0.00671 | 1 | 149 | 38 | 1 | ATG −> ACG | M −> T | NS2A |
| 3700 | G | A | 0.99367 | 0.00633 | 1 | 158 | 52 | 1 | AGT −> AAT | S −> N | NS2A |
| 3718 | C | T | 0.99367 | 0.00633 | 1 | 158 | 58 | 1 | GCA −> GTA | A −> V | NS2A |
| 3771 | C | T | 0.99363 | 0.00637 | 1 | 157 | 76 | 1 | CAT −> TAT | H −> Y | NS2A |
| 3784 | T | C | 0.99333 | 0.00667 | 1 | 150 | 80 | 1 | ATA −> ACA | I −> T | NS2A |
| 3882 | C | T | 0.99333 | 0.00667 | 1 | 150 | 113 | 1 | CTT −> TTT | L −> F | NS2A |
| 3925 | T | C | 0.98639 | 0.01361 | 2 | 147 | 127 | 1 | GTT −> GCT | V −> A | NS2A |
| 3993 | A | G | 0.99338 | 0.00662 | 1 | 151 | 150 | 1 | ATC −> GTC | I −> V | NS2A |
| 3999 | T | A | 0.96026 | 0.00662 | 1 | 151 | 152 | 2 | TTG −> ATG | L −> M | NS2A |
| 4029 | G | A | 0.99342 | 0.00658 | 1 | 152 | 162 | 1 | GCC −> ACC | A −> T | NS2A |
| 4030 | C | T | 0.98684 | 0.01316 | 2 | 152 | 162 | 1 | GCC −> GTC | A −> V | NS2A |
| 4047 | G | A | 0.99342 | 0.00658 | 1 | 152 | 168 | 2 | GTG −> ATG | V −> M | NS2A |
| 4113 | A | G | 0.99259 | 0.00741 | 1 | 135 | 190 | 1 | AGT −> GGT | S −> G | NS2A |
| 4213 | G | A | 0.99265 | 0.00735 | 1 | 136 | 223 | 1 | AGT −> AAT | S −> N | NS2A |
| 4287 | G | A | 0.93103 | 0.06897 | 10 | 145 | 22 | 1 | GCC −> ACC | A −> T | NS2B |
| 4319 | G | A | 0.99315 | 0.00685 | 1 | 146 | 32 | 3 | ATG −> ATA | M −> I | NS2B |
| 4363 | A | G | 0.99329 | 0.00671 | 1 | 149 | 47 | 1 | AAG −> AGG | K −> R | NS2B |
| 4536 | A | G | 0.9918 | 0.0082 | 1 | 122 | 105 | 1 | ACC −> GCC | T −> A | NS2B |
| 4651 | T | C | 0.98701 | 0.01299 | 2 | 154 | 13 | 1 | GTA −> GCA | V −> A | NS3 |
| 4663 | A | G | 0.98684 | 0.01316 | 2 | 152 | 17 | 1 | GAG −> GGG | E −> G | NS3 |
| 4956 | A | G | 0.9863 | 0.0137 | 2 | 146 | 115 | 2 | ATA −> GTA | I −> V | NS3 |
| 4957 | T | C | 0.99315 | 0.00685 | 1 | 146 | 115 | 1 | ATA −> ACA | I −> T | NS3 |
| 4972 | A | G | 0.97778 | 0.02222 | 3 | 135 | 120 | 1 | GAT −> GGT | D −> G | NS3 |
| 4989 | G | A | 0.99107 | 0.00893 | 1 | 112 | 126 | 1 | GTT −> ATT | V −> I | NS3 |
| 5038 | A | G | 0.99107 | 0.00893 | 1 | 112 | 142 | 1 | AAG −> AGG | K −> R | NS3 |
| 5232 | G | A | 0.99291 | 0.00709 | 1 | 141 | 207 | 1 | GAA −> AAA | E −> K | NS3 |
| 5614 | T | C | 0.98529 | 0.01471 | 2 | 136 | 334 | 1 | ATG −> ACG | M −> T | NS3 |
| 5671 | C | T | 0.99286 | 0.00714 | 1 | 140 | 353 | 1 | ACG −> ATG | T −> M | NS3 |
| 5676 | C | T | 0.97183 | 0.02817 | 4 | 142 | 355 | 1 | CAT −> TAT | H −> Y | NS3 |
| 5832 | G | T | 0.99367 | 0.00633 | 1 | 158 | 407 | 2 | GTG −> TTG | V −> L | NS3 |
| 6106 | A | G | 0.99351 | 0.00649 | 1 | 154 | 498 | 1 | TAC −> TGC | Y −> C | NS3 |
| 6187 | G | A | 0.99342 | 0.00658 | 1 | 152 | 525 | 1 | AGG −> AAG | R −> K | NS3 |
| 6223 | A | G | 0.98684 | 0.01316 | 2 | 152 | 537 | 1 | AAA −> AGA | K −> R | NS3 |
| 6277 | C | T | 0.99346 | 0.00654 | 1 | 153 | 555 | 1 | ACC −> ATC | T −> I | NS3 |
| 6306 | G | A | 0.99342 | 0.00658 | 1 | 152 | 565 | 1 | GGC −> AGC | G −> S | NS3 |
| 6310 | C | T | 0.99342 | 0.00658 | 1 | 152 | 566 | 1 | ACG −> ATG | T −> M | NS3 |
| 6327 | A | C | 0.90132 | 0.09868 | 15 | 152 | 572 | 2 | ATG −> CTG | M −> L | NS3 |
| 6364 | A | G | 0.99338 | 0.00662 | 1 | 151 | 584 | 1 | CAC −> CGC | H −> R | NS3 |
| 6453 | G | T | 0.9871 | 0.0129 | 2 | 155 | 614 | 1 | GCT −> TCT | A −> S | NS3 |
| 6468 | G | A | 0.99338 | 0.00662 | 1 | 151 | 2 | 1 | GCG −> ACG | A −> T | NS4A |
| 6471 | G | A | 0.99324 | 0.00676 | 1 | 148 | 3 | 1 | GCT −> ACT | A −> T | NS4A |
| 6474 | T | C | 0.97917 | 0.02083 | 3 | 144 | 4 | 1 | TTT −> CTT | F −> L | NS4A |
| 6481 | T | A | 0.99301 | 0.00699 | 1 | 143 | 6 | 1 | GTG −> GAG | V −> E | NS4A |
| 6485 | G | C | 0.99301 | 0.00699 | 1 | 143 | 7 | 3 | ATG −> ATC | M −> I | NS4A |
| 6487 | A | G | 0.99301 | 0.00699 | 1 | 143 | 8 | 1 | GAA −> GGA | E −> G | NS4A |
| 6592 | C | T | 0.9931 | 0.0069 | 1 | 145 | 43 | 1 | GCC −> GTC | A −> V | NS4A |
| 6598 | C | T | 0.99315 | 0.00685 | 1 | 146 | 45 | 1 | GCG −> GTG | A −> V | NS4A |
| 6606 | T | A | 0.95804 | 0.04196 | 6 | 143 | 48 | 2 | TTG −> ATG | L −> M | NS4A |
| 6866 | G | A | 0.99338 | 0.00662 | 1 | 151 | 7 | 3 | ATG −> ATA | M −> I | 2K |
| 6889 | T | C | 0.99338 | 0.00662 | 1 | 151 | 15 | 1 | GTA −> GCA | V −> A | 2K |
| 6894 | C | T | 0.99338 | 0.00662 | 1 | 151 | 17 | 1 | CTT −> TTT | L −> F | 2K |
| 6906 | A | G | 0.99338 | 0.00662 | 1 | 151 | 21 | 1 | ATT −> GTT | I −> V | 2K |
| 6934 | A | G | 0.99338 | 0.00662 | 1 | 151 | 7 | 1 | GAG −> GGG | E −> G | NS4B |
| 6957 | C | T | 0.99333 | 0.00667 | 1 | 150 | 15 | 1 | CAT −> TAT | H −> Y | NS4B |
| 6963 | A | C | 0.99333 | 0.00667 | 1 | 150 | 17 | 2 | ATG −> CTG | M −> L | NS4B |
| 6970 | G | A | 0.99333 | 0.00667 | 1 | 150 | 19 | 1 | AGG −> AAG | R −> K | NS4B |
| 6984 | G | A | 0.99333 | 0.00667 | 1 | 150 | 24 | 1 | GCA −> ACA | A −> T | NS4B |
| 6991 | T | C | 0.99346 | 0.00654 | 1 | 153 | 26 | 1 | ATA −> ACA | I −> T | NS4B |
| 6992 | A | G | 0.96078 | 0.03922 | 6 | 153 | 26 | 3 | ATA −> ATG | I −> M | NS4B |
| 6996 | T | A | 0.99346 | 0.00654 | 1 | 153 | 28 | 1 | TTC −> ATC | F −> I | NS4B |
| 7206 | A | G | 0.99291 | 0.00709 | 1 | 141 | 98 | 2 | ATA −> GTA | I −> V | NS4B |
| 7208 | A | G | 0.99291 | 0.00709 | 1 | 141 | 98 | 3 | ATA −> ATG | I −> M | NS4B |
| 7395 | A | G | 0.99286 | 0.00714 | 1 | 140 | 161 | 1 | ACA −> GCA | T −> A | NS4B |
| 7441 | T | C | 0.99254 | 0.00746 | 1 | 134 | 176 | 1 | ATA −> ACA | I −> T | NS4B |
| 7442 | A | G | 0.97761 | 0.02239 | 3 | 134 | 176 | 3 | ATA −> ATG | I −> M | NS4B |
| 7471 | C | T | 0.99248 | 0.00752 | 1 | 133 | 186 | 1 | TCG −> TTG | S −> L | NS4B |
| 7519 | C | G | 0.99265 | 0.00735 | 1 | 136 | 202 | 1 | GCA −> GGA | A −> G | NS4B |
| 7633 | C | T | 0.9931 | 0.0069 | 1 | 145 | 240 | 1 | ACA −> ATA | T −> I | NS4B |
| 7690 | T | C | 0.99296 | 0.00704 | 1 | 142 | 8 | 1 | CTG −> CCG | L −> P | NS5 |
| 7743 | T | C | 0.99259 | 0.00741 | 1 | 135 | 26 | 1 | TCC −> CCC | S −> P | NS5 |
| 7755 | T | A | 0.9927 | 0.0073 | 1 | 137 | 30 | 1 | TCA −> ACA | S −> T | NS5 |
| 7773 | T | A | 0.99275 | 0.00725 | 1 | 138 | 36 | 1 | TGC −> AGC | C −> S | NS5 |
| 7824 | C | T | 0.99259 | 0.00741 | 1 | 135 | 53 | 1 | CAT −> TAT | H −> Y | NS5 |
| 7887 | T | C | 0.96575 | 0.03425 | 5 | 146 | 74 | 1 | TAT −> CAT | Y −> H | NS5 |
| 7939 | C | T | 0.9931 | 0.0069 | 1 | 145 | 91 | 1 | GCC −> GTC | A −> V | NS5 |
| 7996 | A | G | 0.99254 | 0.00746 | 1 | 134 | 110 | 1 | CAT −> CGT | H −> R | NS5 |
| 8070 | A | G | 0.99259 | 0.00741 | 1 | 135 | 135 | 2 | ATG −> GTG | M −> V | NS5 |
| 8082 | C | T | 0.98519 | 0.01481 | 2 | 135 | 139 | 1 | CCG −> TCG | P −> S | NS5 |
| 8170 | T | C | 0.99259 | 0.00741 | 1 | 135 | 168 | 1 | GTG −> GCG | V −> A | NS5 |
| 8188 | A | G | 0.98621 | 0.01379 | 2 | 145 | 174 | 1 | AAA −> AGA | K −> R | NS5 |
| 8250 | C | A | 0.94702 | 0.01325 | 2 | 151 | 195 | 2 | CTG −> ATG | L −> M | NS5 |
| 8271 | T | C | 0.97351 | 0.02649 | 4 | 151 | 202 | 1 | TAT −> CAT | Y −> H | NS5 |
| 8403 | G | T | 0.9931 | 0.0069 | 1 | 145 | 246 | 2 | GGG −> TGG | G −> W | NS5 |
| 8406 | C | T | 0.99306 | 0.00694 | 1 | 144 | 247 | 1 | CCT −> TCT | P −> S | NS5 |
| 8439 | A | G | 0.99306 | 0.00694 | 1 | 144 | 258 | 1 | AAT −> GAT | N −> D | NS5 |
| 8467 | T | C | 0.98101 | 0.01899 | 3 | 158 | 267 | 1 | GTA −> GCA | V −> A | NS5 |
| 8473 | G | A | 0.99367 | 0.00633 | 1 | 158 | 269 | 1 | TGC −> TAC | C −> Y | NS5 |
| 8505 | A | G | 0.99367 | 0.00633 | 1 | 158 | 280 | 1 | AAC −> GAC | N −> D | NS5 |
| 8550 | T | G | 0.98734 | 0.01266 | 2 | 158 | 295 | 1 | TTT −> GTT | F −> V | NS5 |
| 8599 | A | T | 0.99324 | 0.00676 | 1 | 148 | 311 | 1 | GAG −> GTG | E −> V | NS5 |
| 8600 | G | T | 0.99324 | 0.00676 | 1 | 148 | 311 | 2 | GAG −> GAT | E −> D | NS5 |
| 8631 | A | G | 0.66892 | 0.33108 | 49 | 148 | 322 | 2 | ATA −> GTA | I −> V | NS5 |
| 8676 | A | G | 0.99333 | 0.00667 | 1 | 150 | 337 | 1 | ACT −> GCT | T −> A | NS5 |
| 8794 | G | C | 0.99371 | 0.00629 | 1 | 159 | 376 | 1 | AGC −> ACC | S −> T | NS5 |
| 8817 | A | C | 0.98742 | 0.01258 | 2 | 159 | 384 | 1 | AAA −> CAA | K −> Q | NS5 |
| 8823 | C | A | 0.99371 | 0.00629 | 1 | 159 | 386 | 2 | CTA −> ATA | L −> I | NS5 |
| 8830 | A | G | 0.98742 | 0.01258 | 2 | 159 | 388 | 1 | AAA −> AGA | K −> R | NS5 |
| 8832 | C | T | 0.99371 | 0.00629 | 1 | 159 | 389 | 1 | CAC −> TAC | H −> Y | NS5 |
| 8980 | A | G | 0.99291 | 0.00709 | 1 | 141 | 438 | 1 | AAG −> AGG | K −> R | NS5 |
| 8991 | C | T | 0.93478 | 0.06522 | 9 | 138 | 442 | 1 | CAC −> TAC | H −> Y | NS5 |
| 8992 | A | G | 0.99291 | 0.00709 | 1 | 141 | 442 | 1 | CAC −> CGC | H −> R | NS5 |
| 9117 | T | C | 0.99333 | 0.00667 | 1 | 150 | 484 | 1 | TTT −> CTT | F −> L | NS5 |
| 9240 | C | T | 0.88281 | 0.10938 | 14 | 128 | 525 | 1 | CGC −> TGC | R −> C | NS5 |
| 9240 | C | A | 0.88281 | 0.00781 | 1 | 128 | 525 | 1 | CGC −> AGC | R −> S | NS5 |
| 9244 | T | C | 0.99219 | 0.00781 | 1 | 128 | 526 | 1 | ATA −> ACA | I −> T | NS5 |
| 9327 | A | G | 0.9375 | 0.0625 | 8 | 128 | 554 | 1 | ATC −> GTC | I −> V | NS5 |
| 9346 | A | G | 0.99231 | 0.00769 | 1 | 130 | 560 | 1 | AAA −> AGA | K −> R | NS5 |
| 9406 | A | G | 0.98649 | 0.01351 | 2 | 148 | 580 | 1 | AAG −> AGG | K −> R | NS5 |
| 9591 | T | C | 0.96992 | 0.03008 | 4 | 133 | 642 | 1 | TCA −> CCA | S −> P | NS5 |
| 9595 | A | G | 0.99248 | 0.00752 | 1 | 133 | 643 | 1 | GAG −> GGG | E −> G | NS5 |
| 9604 | C | A | 0.99248 | 0.00752 | 1 | 133 | 646 | 1 | ACC −> AAC | T −> N | NS5 |
| 9616 | A | G | 0.98496 | 0.01504 | 2 | 133 | 650 | 1 | CAG −> CGG | Q −> R | NS5 |
| 9619 | G | A | 0.99248 | 0.00752 | 1 | 133 | 651 | 1 | AGC −> AAC | S −> N | NS5 |
| 9627 | T | C | 0.98496 | 0.01504 | 2 | 133 | 654 | 2 | TGG −> CGG | W −> R | NS5 |
| 9775 | A | T | 0.99167 | 0.00833 | 1 | 120 | 703 | 1 | GAC −> GTC | D −> V | NS5 |
| 9840 | T | C | 0.9916 | 0.0084 | 1 | 119 | 725 | 1 | TCC −> CCC | S −> P | NS5 |
| 10090 | C | T | 0.85211 | 0.14789 | 21 | 142 | 808 | 1 | ACT −> ATT | T −> I | NS5 |
| 10101 | C | A | 0.99286 | 0.00714 | 1 | 140 | 812 | 1 | CTT −> ATT | L −> I | NS5 |
| 10155 | A | G | 0.99265 | 0.00735 | 1 | 136 | 830 | 1 | ACC −> GCC | T −> A | NS5 |
| 10164 | A | G | 0.91241 | 0.08759 | 12 | 137 | 833 | 1 | ACG −> GCG | T −> A | NS5 |
| 10165 | C | T | 0.9927 | 0.0073 | 1 | 137 | 833 | 1 | ACG −> ATG | T −> M | NS5 |
| 10221 | C | T | 0.99265 | 0.00735 | 1 | 136 | 852 | 1 | CTC −> TTC | L −> F | NS5 |
| 10295 | A | G | 0.98529 | 0.01471 | 2 | 136 | 876 | 3 | ATA −> ATG | I −> M | NS5 |
| 10301 | T | G | 0.65185 | 0.34815 | 47 | 135 | 878 | 2 | GAT −> GAG | D −> E | NS5 |
| 10315 | T | C | 0.99259 | 0.00741 | 1 | 135 | 883 | 1 | ATG −> ACG | M −> T | NS5 |

Table 5 below provides substitution rates across the 164 genomes analyzed (100 of which were sequenced as part of this study). Includes observed mutations per available base as well as substitution rates estimated by BEAST.

| | Observed rate, per available base | | | | | | Mean rate (BEAST), substitutions/site/year | | | |
|---|---|---|---|---|---|---|---|---|---|---|
| | | 1st | 2nd | 3rd | | 4-fold | 1st | 2nd | 3rd | 5′ and |
| | All | codon pos | codon pos | codon pos | 3′ UTR | deg sites | codon pos | codon pos | codon pos | 3′ UTRs |
| A > C | 4.39E−03 | 4.78E−03 | 0.00E+00 | 6.94E−03 | 0.00E+00 | 1.26E−02 | 3.86E−05 | 1.57E−05 | 1.05E−04 | 2.74E−04 |
| C > A | 8.80E−03 | 6.78E−03 | 2.55E−03 | 1.59E−02 | 0.00E+00 | 2.08E−02 | 6.83E−05 | 1.79E−05 | 1.03E−04 | 2.80E−04 |
| A > T | 9.12E−03 | 2.87E−03 | 3.31E−03 | 2.31E−02 | 8.85E−03 | 3.58E−02 | 4.21E−05 | 1.98E−05 | 1.83E−04 | 1.26E−04 |
| T > A | 1.08E−02 | 1.26E−02 | 1.03E−03 | 2.37E−02 | 0.00E+00 | 3.78E−02 | 7.85E−05 | 1.83E−05 | 2.33E−04 | 1.72E−04 |
| C > G | 8.38E−04 | 0.00E+00 | 1.28E−03 | 0.00E+00 | 0.00E+00 | 0.00E+00 | 4.02E−07 | 2.96E−05 | 2.40E−05 | 3.11E−04 |
| G > C | 3.49E−03 | 0.00E+00 | 5.27E−03 | 4.97E−03 | 0.00E+00 | 9.98E−03 | 1.93E−07 | 3.05E−05 | 2.08E−05 | 2.65E−04 |
| G > T | 4.76E−03 | 3.25E−03 | 1.32E−03 | 8.95E−03 | 0.00E+00 | 1.75E−02 | 1.82E−05 | 6.19E−06 | 8.13E−05 | 1.85E−04 |
| T > G | 5.20E−03 | 3.59E−03 | 0.00E+00 | 1.33E−02 | 0.00E+00 | 2.33E−02 | 4.01E−05 | 4.88E−06 | 1.21E−04 | 2.89E−04 |
| A > G | 5.91E−02 | 2.20E−02 | 2.65E−02 | 1.40E−01 | 5.31E−02 | 1.35E−01 | 1.83E−04 | 1.84E−04 | 1.38E−03 | 1.01E−03 |
| G > A | 4.50E−02 | 1.22E−02 | 1.19E−02 | 1.13E−01 | 2.33E−02 | 1.55E−01 | 1.55E−04 | 2.16E−04 | 1.17E−03 | 8.78E−04 |
| C > T | 1.22E−01 | 7.46E−02 | 2.42E−02 | 2.53E−01 | 4.20E−02 | 2.42E−01 | 8.09E−04 | 2.99E−04 | 2.68E−03 | 9.36E−04 |
| T > C | 1.19E−01 | 7.90E−02 | 2.67E−02 | 2.93E−01 | 8.96E−02 | 3.17E−01 | 8.52E−04 | 2.43E−04 | 3.47E−03 | 1.24E−03 |

TABLE 6
Sequences used in the present study. R refers to A or G; Y refers to C or T;
S refers to G or C; W refers to A or T.

| | Assay Forward | Assay Reverse | | Amplicon |
|---|---|---|---|---|
| qPCR Assay | Primer | Primer | Assay PCR-Probe | (Target Sequence) |
| Zika | GGCTTG | CCCTCAATG | AGATGGCCTC | GGCTTGAAGCAAGAATGC |
| | AAGCAA | GCTGCTACTTT | ATAGCCTCGCTCTA | TCCTTGACAATATTTACCTC |
| | GAATGC TC | (Seq. I.D. No. 4) | (Seq. I.D. No. 5) | CAAGATGGCCTCATAGCCT |
| | (Seq. ID. No. 3) | | | CGCTCTATCGACCTGAGGC |
| | | | | CGACAAAGTAGCAGCCAT |
| | | | | TGAGGG |
| | | | | (Seq. I.D. No. 6) |
| Zika | ATTGAGGA | GTTCTTTCCT | AAGACGGCTG | ATTGAGGAATGGTGCTGTA |
| | ATGGTGCT | GGGCCTTATCT | CTGGTATGGAATGG | GGGAATGCACAATGCCCC |
| | GTAGG | (Seq. I.D. No. 8) | (Seq. I.D. No. 9) | CACTATCGTTTCGAGCAAA |
| | (Seq. I.D. No. 7) | | | AGACGGCTGCTGGTATGG |
| | | | | AATGGAGATAAGGCCCAG |
| | | | | GAAAGAAC |
| | | | | (Seq. I.D. No. 10) |
| Zika | TCATGAAG | CTCAGCCGC | TGCAAAGCTATGGG | TCATGAAGAACCCGTGTTG |
| | AACCCRTG | CATRTGRAA | TGGAACA | GTGCAAAGCTATGGGTGG |
| | YTGG | GA | (Seq. I.D. No. 13) | AACATAGTCCGTCTTAAGA |
| | (Seq. I.D. No. 11) | (Seq. I.D. No. 12) | | GTGGGGTGGACGTCTTTC |
| | | | | ATATGGCGGCTGAG |
| | | | | (Seq. I.D. No. 14) |
| Zika | AGYYGAYT | YTCCTCAATC | ACCTGGTCAATCCA | AGTTGACTGGGTTCCAACT |
| | GGGTHCCA | CACACTCTRT | TGGAAAGGGA | GGGAGAACTACCTGGTCA |
| | AC TG | TC | (Seq. I.D. No. 17) | ATCCATGGAAAGGGAGAA |
| | (Seq. I.D. No. 15) | (Seq. I.D. No. 16) | | TGGATGACCACTGAAGAC |
| | | | | ATGCTTGTGGTGTGGAACA |
| | | | | GAGTGTGGATTGAGGAG |
| | | | | (Seq. I.D. No. 18) |
| Zika | CCAYTTCA | TTTGCWARC | TGCCGCCACCAAGA | CCACTTCAACAAGCTCCAT |
| | ACAARCTS | ARGCAGTCTC | TGAACTGA | CTCAAGGACGGGAGGTCC |
| | YAYCT | (SEQ. I.D. No. 20) | (SEQ. I.D. No. 21) | ATTGTGGTTCCCTGCCGCC |
| | (SEQ. I.D. No. 19) | | | ACCAAGATGAACTGATTG |
| | | | | GCCGGGCCCGCGTCTCTCC |
| | | | | AGGGGCGGGATGGAGCAT |
| | | | | CCGGGAGACTGCTTGCCTA |
| | | | | GCAAA |
| | | | | (SEQ. I.D. No. 22) |
| Zika | TSYAGGGA | ACTAAGTTR | TGGTATGGAATGGA | AGGGAGTGCACAATGCCC |
| | RTGCACAAT | CTYTCTGGTT | GATAAGGCCC | CCACTGTCGTTCCGGGCTA |
| | (SEQ. I.D. No. 23) | CYTTY | (SEQ. I.D. No. 25) | AAGATGGCTGTTGGTATGG |
| | | (SEQ. I.D. No. 24) | | AATGGAGATAAGGCCCAG |
| | | | | GAAAGAACCAGAAAGCAA |
| | | | | CTTAGTAAGG |
| | | | | (SEQ. I.D. No. 26) |
| Zika | AGAGACCC | CTCGGTGAT | AGATGTCGGC | AGAGACCCTGGGAGAGAA |
| | TGGGAGAG | GCCTGA | CCTGGAGTT CTACT | ATGGAAGGCCCGCTTGAA |
| | AA AT | CTTT | (SEQ. I.D. No. 29) | CCAGATGTCGGCCCTGGA |
| | (SEQ. I.D. No. 27) | (SEQ I.D. No. 28) | | GTTCTACTCCTACAAAAAG |
| | | | | TCAGGCATCACCGAG |
| | | | | (SEQ. I.D. No. 30) |
| Chikungunya | TTTGCAAG | GTAGCTGTA | GAGAAGCTCAGAG | TTTGCAAGCTCCAGATCCA |
| | CTCCAGAT | GTGCGTACC | GACCCGT | ACTTCGAGAAGCTCAGAG |
| | CCA | TATTT | (SEQ. I.D. No. 33) | GACCCGTCATAACTTTGTA |
| | (SEQ I.D. No. 31) | (SEQ I.D. No. 32) | | CGGCGGTCCTAAATAGGTA |
| | | | | CGCACTACAGCTAC |
| | | | | (SEQ. I.D. No. 34) |
| Chikungunya | CGTTCTCG | TGATCCCGA | GTACTTCCTGTCCG | CGTTCTCGCATCTAGCCAT |
| | CATCTAGC | CTCAACCAT | ACATCATC | AAAACTAATAGAGCAGGA |
| | CATAA | CCTGG | (SEQ. I.D. No. 37) | AATTGATCCCGACTCAACC |
| | (SEQ. I.D. No. 35) | (SEQ. I.D. No. 36) | | ATCCTGGATATAGGTAGTG |
| | | | | CGCCAGCAAGGAGGATGA |
| | | | | TGTCGGACAGGAAGTAC |
| | | | | (SEQ. I.D. No. 38) |
| Chikungunya | CCCGACTC | GCAGACGCA | CCAGCAAGG A | CCCCGACTCAACCATCCTG |
| | AACCATCC | GTGGTACTT | GGATGATGT | GATATCGGCAGTGCGCCAG |
| | TG | (SEQ. I.D. No. 40) | CGG | CAAGGAGGATGATGTCGG |
| | (SEQ I.D. No. 39) | | (SEQ. I.D. No. 41) | ACAGGAAGTACCAGGAAG |
| | | | | TACCACTGCGTCTGCC |
| | | | | (SEQ. I.D. No. 42) |
| Dengue | AACCWAC | GRGAAWCTC | TCAATATGCTG | AACCTACGAAAAAAGACG |
| | GRAARAAG | TTYGYYARC | AAACGC | GCTCGACCGTCTTTCAATA |
| | RCGV | TG | (SEQ. I.D. No. 45) | TGCTGAAACGCGCGAGAA |
| | (SEQ. I.D. No. 43) | (SEQ. I.D. No. 44) | | ACCGCGTGTCAACTGTTTC |
| | | | | ACAGTTGGCGAAGAGATT |
| | | | | CTC |
| | | | | (SEQ. I.D. No. 46) |
| Dengue | Same as | Same as listed | CG TCT TTC AA TAT | Same as listed above |
| | listed above | above | GCT GAA ACG CGC | |
| | | | (SEQ. I.D. No. 47) | |

TABLE 7
Table of information on 229 samples sequenced in this study, including the 110 whose genomes analyzed.

| | | | | Number of | | | | Number of |
|---|---|---|---|---|---|---|---|---|
| | Has | | Number of | passing | | Number of | Number of | passing |
| | passing | | amplicon | amplicon | Number of | passing | hybrid | hybrid |
| | ZIKV | GenBank | sequencing | sequencing | unbiased | unbiased | capture | capture |
| Sample name | assembly | accession | replicates | replicates | replicates | replicates | replicates | replicates |
| BLM_2016_MA-WGS16-006-SER | FALSE | KY829115 | 1 | 0 | 1 | 0 | 1 | 0 |
| | | (DENV) | | | | | | |
| BRA_2016_FC-28209-CSF | FALSE | | 1 | 0 | 0 | 0 | 0 | 0 |
| BRA_2016_FC-546939-SER | FALSE | | 1 | 0 | 0 | 0 | 0 | 0 |
| BRA_2016_FC-547184-SER | FALSE | | 1 | 0 | 0 | 0 | 0 | 0 |
| BRA_2016_FC-547184-URI | FALSE | | 1 | 0 | 0 | 0 | 0 | 0 |
| BRA_2016_FC-547220-SER | FALSE | | 1 | 0 | 0 | 0 | 0 | 0 |
| BRA_2016_FC-547224-URI | FALSE | | 1 | 0 | 0 | 0 | 0 | 0 |
| BRA_2016_FC-547231-URI | FALSE | | 1 | 0 | 0 | 0 | 0 | 0 |
| BRA_2016_FC-5790-SER | TRUE | KY785446 | 1 | 1 | 0 | 0 | 0 | 0 |
| BRA_2016_FC-5905-SER | TRUE | KY014308 | 1 | 1 | 0 | 0 | 0 | 0 |
| BRA_2016_FC-6020-SOCO-SER | FALSE | | 1 | 0 | 0 | 0 | 0 | 0 |
| BRA_2016_FC-6095-SOCO-SER | FALSE | | 1 | 0 | 0 | 0 | 0 | 0 |
| BRA_2016_FC-6284-RNA-SER | FALSE | | 1 | 0 | 0 | 0 | 0 | 0 |
| BRA_2016_FC-6418-SER | TRUE | KY785410 | 3 | 2 | 0 | 0 | 1 | 0 |
| BRA_2016_FC-6696-SER | TRUE | KY014313 | 3 | 2 | 0 | 0 | 0 | 0 |
| BRA_2016_FC-6703-SER | TRUE | KY785439 | 2 | 2 | 0 | 0 | 0 | 0 |
| BRA_2016_FC-6706-SER | TRUE | KY785433 | 3 | 2 | 0 | 0 | 1 | 0 |
| BRA_2016_FC-6863-SER | TRUE | KY785450 | 2 | 2 | 2 | 0 | 1 | 0 |
| BRA_2016_FC-6864-URI | TRUE | KY014297 | 2 | 2 | 1 | 0 | 1 | 1 |
| BRA_2016_FC-DQ105D1-PLA | FALSE | | 2 | 0 | 0 | 0 | 0 | 0 |
| BRA_2016_FC-DQ107D1-URI | TRUE | KY014301 | 2 | 2 | 2 | 0 | 3 | 0 |
| BRA_2016_FC-DQ109D1-PLA | FALSE | | 1 | 0 | 0 | 0 | 0 | 0 |
| BRA_2016_FC-DQ116D1-PLA | TRUE | KY785429 | 3 | 1 | 0 | 0 | 0 | 0 |
| BRA_2016_FC-DQ119D1-URI | FALSE | | 2 | 0 | 0 | 0 | 0 | 0 |
| BRA_2016_FC-DQ121D1-PLA | FALSE | | 2 | 0 | 1 | 0 | 1 | 0 |
| BRA_2016_FC-DQ122D1-PLA | TRUE | KY785456 | 2 | 2 | 0 | 0 | 1 | 0 |
| BRA_2016_FC-DQ125D1-PLA | FALSE | | 2 | 0 | 0 | 0 | 0 | 0 |
| BRA_2016_FC-DQ12D1-PLA | TRUE | KY785436 | 1 | 1 | 0 | 0 | 0 | 0 |
| BRA_2016_FC-DQ131D1-URI | TRUE | KY014296 | 2 | 2 | 4 | 3 | 4 | 4 |
| BRA_2016_FC-DQ14D1-PLA | FALSE | | 1 | 0 | 0 | 0 | 0 | 0 |
| BRA_2016_FC-DQ192D1-URI | TRUE | KY785485 | 4 | 2 | 0 | 0 | 0 | 0 |
| BRA_2016_FC-DQ194D1-URI | FALSE | | 1 | 0 | 0 | 0 | 0 | 0 |
| BRA_2016_FC-DQ197D1-URI | FALSE | | 3 | 0 | 0 | 0 | 0 | 0 |
| BRA_2016_FC-DQ203D1-PLA | FALSE | | 1 | 0 | 0 | 0 | 0 | 0 |
| BRA_2016_FC-DQ219D1-URI | FALSE | | 3 | 0 | 0 | 0 | 0 | 0 |
| BRA_2016_FC-DQ220D1-PLA | FALSE | | 1 | 0 | 0 | 0 | 0 | 0 |
| BRA_2016_FC-DQ246D1-URI | FALSE | | 1 | 0 | 0 | 0 | 0 | 0 |
| BRA_2016_FC-DQ28D1-URI | TRUE | KY014317 | 3 | 2 | 3 | 1 | 3 | 3 |
| BRA_2016_FC-DQ42D1-URI | TRUE | KY014320 | 3 | 3 | 4 | 1 | 4 | 3 |
| BRA_2016_FC-DQ47D1-PLA | TRUE | KY014309 | 1 | 1 | 0 | 0 | 0 | 0 |
| BRA_2016_FC-DQ49D1-PLA | TRUE | KY014307 | 2 | 2 | 2 | 0 | 3 | 0 |
| BRA_2016_FC-DQ52D1-PLA | FALSE | | 1 | 0 | 0 | 0 | 0 | 0 |
| BRA_2016_FC-DQ58D1-PLA | TRUE | KY785427 | 2 | 2 | 0 | 0 | 1 | 0 |
| BRA_2016_FC-DQ5D1-URI | TRUE | KY785437 | 2 | 2 | 0 | 0 | 0 | 0 |
| BRA_2016_FC-DQ60D1-PLA | TRUE | KY785411 | 1 | 1 | 0 | 0 | 0 | 0 |
| BRA_2016_FC-DQ60D1-URI | TRUE | KY785479 | 2 | 2 | 1 | 0 | 1 | 1 |
| BRA_2016_FC-DQ62D1-PLA | TRUE | KY785480 | 2 | 2 | 0 | 0 | 0 | 0 |
| BRA_2016_FC-DQ62D1-URI | FALSE | | 2 | 0 | 2 | 0 | 0 | 0 |
| BRA_2016_FC-DQ62D2-PLA | FALSE | | 2 | 0 | 0 | 0 | 0 | 0 |
| BRA_2016_FC-DQ62D2-URI | TRUE | KY785455 | 1 | 1 | 2 | 2 | 2 | 2 |
| BRA_2016_FC-DQ68D1-URI | TRUE | KY785467 | 2 | 1 | 0 | 0 | 0 | 0 |
| BRA_2016_FC-DQ75D1-PLA | TRUE | KY785409 | 3 | 1 | 0 | 0 | 0 | 0 |
| BRA_2016_FC-DQ75D1-URI | TRUE | KY785426 | 2 | 2 | 1 | 0 | 1 | 1 |
| BRA_2016_FC-DQ77D1-URI | FALSE | | 2 | 0 | 0 | 0 | 0 | 0 |
| COL_2016_SU-1155A-SER | FALSE | | 1 | 0 | 0 | 0 | 0 | 0 |
| COL_2016_SU-1194A-SER | FALSE | | 1 | 0 | 0 | 0 | 0 | 0 |
| COL_2016_SU-1638A-SER | FALSE | | 1 | 0 | 0 | 0 | 0 | 0 |
| COL_2016_SU-1810A-SER | TRUE | KY785417 | 1 | 1 | 0 | 0 | 0 | 0 |
| COL_2016_SU-1856A-SER | TRUE | KY785455 | 1 | 1 | 0 | 0 | 1 | 1 |
| COL_2016_SU-1857A-SER | FALSE | | 1 | 0 | 0 | 0 | 0 | 0 |
| COL_2016_SU-1898A-SER | FALSE | | 1 | 0 | 0 | 0 | 0 | 0 |
| COL_2016_SU-1938A-SER | FALSE | | 1 | 0 | 0 | 0 | 0 | 0 |
| COL_2016_SU-2293A-SER | TRUE | KY785477 | 1 | 1 | 0 | 0 | 0 | 0 |
| COL_2016_SU-2514A-SER | FALSE | | 1 | 0 | 0 | 0 | 0 | 0 |
| COL_2016_SU-2636A-SER | FALSE | | 1 | 0 | 0 | 0 | 0 | 0 |
| COL_2016_SU-2686A-SER | FALSE | | 1 | 0 | 0 | 0 | 0 | 0 |
| COL_2016_SU-2724A-SER | TRUE | KY785469 | 1 | 1 | 0 | 0 | 1 | 1 |
| COL_2016_SU-2836A-SER | FALSE | | 1 | 0 | 0 | 0 | 0 | 0 |
| COL_2016_SU-3117A-SER | FALSE | | 1 | 0 | 0 | 0 | 0 | 0 |
| COL_2016_SU-3311A-SER | FALSE | | 1 | 0 | 0 | 0 | 0 | 0 |
| COL_2016_SU-3315A-SER | FALSE | | 1 | 0 | 0 | 0 | 0 | 0 |
| COL_2016_SU-3316A-SER | FALSE | | 1 | 0 | 0 | 0 | 0 | 0 |
| COL_2016_SU-3351A-SER | FALSE | | 1 | 0 | 0 | 0 | 0 | 0 |
| COL_2016_SU-3407A-SER | FALSE | | 1 | 0 | 0 | 0 | 0 | 0 |
| CUB_2016_FL-011-URI | FALSE | | 1 | 0 | 0 | 0 | 0 | 0 |
| DOM_2016_BB-0054-SER | FALSE | | 1 | 0 | 0 | 0 | 0 | 0 |
| DOM_2016_BB-0054-URI | FALSE | | 1 | 0 | 0 | 0 | 0 | 0 |
| DOM_2016_BB-0059-SER | TRUE | KY785425 | 2 | 2 | 0 | 0 | 0 | 0 |
| DOM_2016_BB-0059-URI | FALSE | | 1 | 0 | 0 | 0 | 0 | 0 |
| DOM_2016_BB-0071-SER | TRUE | KY785463 | 2 | 2 | 0 | 0 | 1 | 0 |
| DOM_2016_BB-0071-URI | TRUE | KY785449 | 1 | 1 | 0 | 0 | 0 | 0 |
| DOM_2016_BB-0076-SER | TRUE | KY014305 | 3 | 3 | 0 | 0 | 1 | 0 |
| DOM_2016_BB-0076-URI | TRUE | KY785423 | 3 | 3 | 0 | 0 | 1 | 0 |
| DOM_2016_BB-0085-SER | TRUE | KY785483 | 1 | 1 | 0 | 0 | 0 | 0 |
| DOM_2016_BB-0085-URI | TRUE | KY785465 | 2 | 2 | 0 | 0 | 0 | 0 |
| DOM_2016_BB-0091-SER | TRUE | KY785475 | 2 | 2 | 0 | 0 | 0 | 0 |
| DOM_2016_BB-0115-SER | TRUE | KY014321 | 3 | 3 | 4 | 2 | 4 | 4 |
| DOM_2016_BB-0115-URI | TRUE | KY785415 | 3 | 3 | 0 | 0 | 0 | 0 |
| DOM_2016_BB-0127-SER | TRUE | KY014303 | 1 | 1 | 3 | 3 | 3 | 3 |
| DOM_2016_BB-0180-SER | TRUE | KY014304 | 4 | 4 | 3 | 3 | 3 | 3 |
| DOM_2016_BB-0180-URI | TRUE | KY785476 | 3 | 3 | 0 | 0 | 1 | 0 |
| DOM_2016_BB-0183-SER | TRUE | KY785420 | 2 | 2 | 0 | 0 | 0 | 0 |
| DOM_2016_BB-0183-URI | FALSE | | 1 | 0 | 0 | 0 | 0 | 0 |
| DOM_2016_BB-0184-URI | FALSE | | 1 | 0 | 0 | 0 | 0 | 0 |
| DOM_2016_BB-0208-SER | TRUE | KY014300 | 1 | 1 | 3 | 2 | 3 | 3 |
| DOM_2016_BB-0216-SER | TRUE | KY014302 | 1 | 1 | 1 | 0 | 1 | 1 |
| DOM_2016_BB-0269-SER | TRUE | KY014318 | 1 | 1 | 1 | 0 | 1 | 1 |
| DOM_2016_BB-0369-SER | FALSE | | 1 | 0 | 0 | 0 | 0 | 0 |
| DOM_2016_BB-0428-PLA | TRUE | KY785428 | 1 | 1 | 0 | 0 | 0 | 0 |
| DOM_2016_BB-0433-SER | TRUE | KY785441 | 2 | 2 | 0 | 0 | 1 | 0 |
| DOM_2016_BB-0436-PLA | TRUE | KY014314 | 1 | 1 | 1 | 0 | 1 | 1 |
| DOM_2016_BB-0439-SER | FALSE | | 1 | 0 | 0 | 0 | 0 | 0 |
| DOM_2016_BB-0445-SER | FALSE | | 1 | 0 | 0 | 0 | 0 | 0 |
| DOM_2016_BB-0448-SER | FALSE | | 1 | 0 | 0 | 0 | 0 | 0 |
| DOM_2016_FL-002-URI | FALSE | | 1 | 0 | 0 | 0 | 0 | 0 |
| DOM_2016_FL-014-URI | FALSE | | 1 | 0 | 0 | 0 | 0 | 0 |
| DOM_2016_MA-WGS16-007-SER | TRUE | KY785453 | 1 | 1 | 0 | 0 | 1 | 1 |
| DOM_2016_MA-WGS16-009-SER | TRUE | KY785478 | 1 | 1 | 0 | 0 | 0 | 0 |
| DOM_2016_MA-WGS16-010-SER | FALSE | | 1 | 0 | 0 | 0 | 0 | 0 |
| DOM_2016_MA-WGS16-011-SER | TRUE | KY785484 | 1 | 1 | 0 | 0 | 1 | 0 |
| DOM_2016_MA-WGS16-013-SER | TRUE | KY785473 | 1 | 1 | 0 | 0 | 0 | 0 |
| DOM_2016_MA-WGS16-014-SER | TRUE | KY785470 | 1 | 1 | 0 | 0 | 1 | 0 |
| DOM_2016_MA-WGS16-020-SER | TRUE | KY785460 | 1 | 1 | 0 | 0 | 0 | 0 |
| DOM_2016_MA-WGS16-021-SER | FALSE | | 1 | 0 | 0 | 0 | 0 | 0 |
| DOM_2016_MA-WGS16-024-SER | TRUE | KY785435 | 1 | 1 | 0 | 0 | 1 | 1 |
| DOM_2016_MA-WGS16-028-SER | FALSE | | 1 | 0 | 0 | 0 | 0 | 0 |
| DOM_2016_MA-WGS16-031-SER | TRUE | KY785434 | 1 | 1 | 0 | 0 | 0 | 0 |
| DOM_2016_MA-WGS16-035-SER | FALSE | | 1 | 0 | 0 | 0 | 0 | 0 |
| DOM_2016_MA-WGS16-036-SER | TRUE | KY785447 | 1 | 1 | 0 | 0 | 0 | 0 |
| DOM_2016_MA-WGS16-040-SER | TRUE | KY785413 | 1 | 1 | 0 | 0 | 0 | 0 |
| GTM_2016_MA-WGS16-026-SER | FALSE | | 1 | 0 | 0 | 0 | 0 | 0 |
| GUY_2016_MA-WGS16-012-SER | FALSE | | 1 | 0 | 0 | 0 | 0 | 0 |
| HND_2016_HU-ME131-PLA | TRUE | KY785414 | 1 | 1 | 0 | 0 | 0 | 0 |
| HND_2016_HU-ME136-PLA | TRUE | KY785461 | 1 | 1 | 0 | 0 | 0 | 0 |
| HND_2016_HU-ME137-PLA | TRUE | KY785444 | 1 | 1 | 0 | 0 | 0 | 0 |
| HND_2016_HU-ME147-SER | TRUE | KY785442 | 2 | 2 | 0 | 0 | 1 | 1 |
| HND_2016_HU-ME149-PLA | FALSE | | 1 | 0 | 0 | 0 | 0 | 0 |
| HND_2016_HU-ME152-SER | TRUE | KY014315 | 2 | 2 | 2 | 0 | 2 | 2 |
| HND_2016_HU-ME156-SER | TRUE | KY785452 | 2 | 2 | 0 | 0 | 1 | 0 |
| HND_2016_HU-ME167-PLA | TRUE | KY014327 | 1 | 1 | 2 | 2 | 2 | 2 |
| HND_2016_HU-ME171-PLA | TRUE | KY785448 | 2 | 2 | 0 | 0 | 1 | 0 |
| HND_2016_HU-ME172-PLA | TRUE | KY785458 | 1 | 1 | 0 | 0 | 0 | 0 |
| HND_2016_HU-ME178-PLA | TRUE | KY014306 | 2 | 2 | 2 | 0 | 2 | 1 |
| HND_2016_HU-ME180-PLA | TRUE | KY785431 | 1 | 1 | 0 | 0 | 0 | 0 |
| HND_2016_HU-ME33-PLA | TRUE | KY785416 | 1 | 1 | 0 | 0 | 0 | 0 |
| HND_2016_HU-ME38-PLA | TRUE | KY014319 | 1 | 1 | 2 | 2 | 2 | 2 |
| HND_2016_HU-ME42-SER | TRUE | KY014310 | 1 | 1 | 2 | 2 | 2 | 2 |
| HND_2016_HU-ME50-PLA | TRUE | KY014311 | 1 | 1 | 0 | 0 | 0 | 0 |
| HND_2016_HU-ME58-PLA | TRUE | KY014312 | 1 | 1 | 2 | 1 | 2 | 2 |
| HND_2016_HU-ME59-PLA | TRUE | KY785418 | 2 | 2 | 0 | 0 | 1 | 1 |
| HND_2016_HU-SZ28-SER | FALSE | | 1 | 0 | 0 | 0 | 0 | 0 |
| HND_2016_HU-SZ76-SER | TRUE | KY785471 | 1 | 1 | 0 | 0 | 0 | 0 |
| HTI_2016_FL-018-URI | FALSE | | 1 | 0 | 0 | 0 | 0 | 0 |
| HTI_2016_MA-WGS16-002-SER | FALSE | | 1 | 0 | 0 | 0 | 0 | 0 |
| HTI_2016_MA-WGS16-003-SER | FALSE | | 1 | 0 | 0 | 0 | 0 | 0 |
| HTI_2016_MA-WGS16-022-SER | TRUE | KY785482 | 1 | 1 | 0 | 0 | 0 | 0 |
| JAM_2016_FL-006-SER | FALSE | | 1 | 0 | 0 | 0 | 0 | 0 |
| JAM_2016_FL-006-URI | FALSE | | 1 | 0 | 0 | 0 | 0 | 0 |
| JAM_2016_FL-007-URI | FALSE | | 1 | 0 | 0 | 0 | 0 | 0 |
| JAM_2016_FL-012-URI | FALSE | | 2 | 0 | 0 | 0 | 0 | 0 |
| JAM_2016_FL-015-URI | FALSE | | 1 | 0 | 0 | 0 | 0 | 0 |
| JAM_2016_MA-WGS16-018-SER | FALSE | | 1 | 0 | 0 | 0 | 0 | 0 |
| JAM_2016_MA-WGS16-025-SER | TRUE | KY785424 | 1 | 1 | 0 | 0 | 1 | 0 |
| JAM_2016_MA-WGS16-037-SER | FALSE | | 1 | 0 | 0 | 0 | 0 | 0 |
| JAM_2016_MA-WGS16-038-SER | TRUE | KY785438 | 1 | 1 | 0 | 0 | 0 | 0 |
| JAM_2016_MA-WGS16-039-SER | TRUE | KY785430 | 1 | 1 | 0 | 0 | 1 | 0 |
| JAM_2016_MA-WGS16-041-SER | TRUE | KY785432 | 1 | 1 | 0 | 0 | 0 | 0 |
| JAM_2016_WI-JM2-SER | FALSE | | 1 | 0 | 0 | 0 | 0 | 0 |
| JAM_2016_WI-JM3A-SER | FALSE | | 1 | 0 | 0 | 0 | 0 | 0 |
| JAM_2016_WI-JM3B-URI | FALSE | | 1 | 0 | 0 | 0 | 0 | 0 |
| JAM_2016_WI-JM4-SER | FALSE | | 1 | 0 | 0 | 0 | 0 | 0 |
| JAM_2016_WI-JM5-SER | FALSE | | 1 | 0 | 0 | 0 | 0 | 0 |
| JAM_2016_WI-JM6-SER | TRUE | KY785419 | 1 | 1 | 0 | 0 | 1 | 0 |
| JAM_2016_WI-JM7A-SER | FALSE | | 1 | 0 | 0 | 0 | 0 | 0 |
| JAM_2016_WI-JM7B-URI | FALSE | | 1 | 0 | 0 | 0 | 0 | 0 |
| JAM_2016_WI-JM8-SER | FALSE | | 1 | 0 | 0 | 0 | 0 | 0 |
| MEX_2016_MA-WGS16-030-SER | FALSE | | 1 | 0 | 0 | 0 | 0 | 0 |
| MTQ_2016_FL-001-SAL | TRUE | KY785451 | 1 | 1 | 0 | 0 | 0 | 0 |
| MTQ_2016_FL-001-SER | FALSE | | 1 | 0 | 0 | 0 | 0 | 0 |
| MTQ_2016_FL-001-URI | FALSE | | 1 | 0 | 0 | 0 | 0 | 0 |
| PRI_2016_FL-004-SER | FALSE | | 1 | 0 | 0 | 0 | 0 | 0 |
| PRI_2016_FL-004-URI | FALSE | | 1 | 0 | 0 | 0 | 0 | 0 |
| PRI_2016_FL-005-SER | FALSE | | 1 | 0 | 0 | 0 | 0 | 0 |
| PRI_2016_FL-005-URI | FALSE | | 1 | 0 | 0 | 0 | 0 | 0 |
| PRI_2016_FL-008-URI | FALSE | | 1 | 0 | 0 | 0 | 0 | 0 |
| PRI_2016_FL-009-URI | FALSE | | 1 | 0 | 0 | 0 | 0 | 0 |
| PRI_2016_FL-013-URI | FALSE | | 1 | 0 | 0 | 0 | 0 | 0 |
| PRI_2016_FL-016-URI | FALSE | | 1 | 0 | 0 | 0 | 0 | 0 |
| PRI_2016_MA-WGS16-004-SER | TRUE | KY785464 | 1 | 1 | 0 | 0 | 1 | 1 |
| PRI_2016_MA-WGS16-005-SER | TRUE | KY785481 | 1 | 1 | 0 | 0 | 0 | 0 |
| PRI_2016_MA-WGS16-015-SER | FALSE | | 1 | 0 | 0 | 0 | 0 | 0 |
| PRI_2016_MA-WGS16-016-SER | TRUE | KY785462 | 1 | 1 | 0 | 0 | 1 | 0 |
| PRI_2016_MA-WGS16-017-SER | FALSE | | 1 | 0 | 0 | 0 | 0 | 0 |
| PRI_2016_MA-WGS16-019-SER | FALSE | | 1 | 0 | 0 | 0 | 0 | 0 |
| PRI_2016_MA-WGS16-032-SER | FALSE | | 1 | 0 | 0 | 0 | 0 | 0 |
| SLV_2016_MA-WGS16-001-SER | FALSE | | 1 | 0 | 0 | 0 | 0 | 0 |
| TTO_2016_FL-003-URI | FALSE | | 2 | 0 | 0 | 0 | 0 | 0 |
| TTO_2016_MA-WGS16-027-SER | FALSE | | 1 | 0 | 0 | 0 | 0 | 0 |
| UNK_2016_MA-WGS16-008-SER | FALSE | | 1 | 0 | 0 | 0 | 0 | 0 |
| UNK_2016_MA-WGS16-023-SER | FALSE | | 1 | 0 | 0 | 0 | 0 | 0 |
| UNK_2016_MA-WGS16-029-SER | TRUE | KY785454 | 1 | 1 | 0 | 0 | 0 | 0 |
| UNK_2016_MA-WGS16-033-SER | FALSE | | 1 | 0 | 0 | 0 | 0 | 0 |
| USA_2016_FL-01-MOS | TRUE | KY014324 | 1 | 1 | 2 | 0 | 2 | 2 |
| USA_2016_FL-010-SER | FALSE | | 1 | 0 | 0 | 0 | 0 | 0 |
| USA_2016_FL-010-URI | TRUE | KY014295 | 1 | 1 | 2 | 0 | 2 | 2 |
| USA_2016_FL-019-URI | TRUE | KY785421 | 1 | 1 | 0 | 0 | 0 | 0 |
| USA_2016_FL-02-MOS | TRUE | KY014323 | 1 | 1 | 2 | 0 | 2 | 2 |
| USA_2016_FL-020-URI | FALSE | | 1 | 0 | 0 | 0 | 0 | 0 |
| USA_2016_FL-021-URI | TRUE | KY785445 | 2 | 2 | 0 | 0 | 1 | 0 |
| USA_2016_FL-022-URI | TRUE | KY785412 | 1 | 1 | 0 | 0 | 0 | 0 |
| USA_2016_FL-023-URI | FALSE | | 1 | 0 | 0 | 0 | 0 | 0 |
| USA_2016_FL-023-WBL | FALSE | | 1 | 0 | 0 | 0 | 0 | 0 |
| USA_2016_FL-025-URI | FALSE | | 1 | 0 | 0 | 0 | 0 | 0 |
| USA_2016_FL-026-URI | FALSE | | 1 | 0 | 0 | 0 | 0 | 0 |
| USA_2016_FL-027-URI | FALSE | | 1 | 0 | 0 | 0 | 0 | 0 |
| USA_2016_FL-028-SER | TRUE | KY785443 | 1 | 1 | 0 | 0 | 0 | 0 |
| USA_2016_FL-028-URI | FALSE | | 2 | 0 | 0 | 0 | 0 | 0 |
| USA_2016_FL-029-URI | TRUE | KY785457 | 2 | 2 | 0 | 0 | 0 | 0 |
| USA_2016_FL-03-MOS | TRUE | KY014322 | 1 | 1 | 2 | 0 | 2 | 2 |
| USA_2016_FL-030-URI | TRUE | KY014325 | 2 | 2 | 0 | 0 | 1 | 0 |
| USA_2016_FL-031-URI | FALSE | | 1 | 0 | 0 | 0 | 0 | 0 |
| USA_2016_FL-032-URI | TRUE | KY014298 | 1 | 1 | 2 | 0 | 2 | 1 |
| USA_2016_FL-033-URI | FALSE | | 2 | 0 | 0 | 0 | 0 | 0 |
| USA_2016_FL-034-SER | FALSE | | 1 | 0 | 0 | 0 | 0 | 0 |
| USA_2016_FL-034-URI | FALSE | | 1 | 0 | 0 | 0 | 0 | 0 |
| USA_2016_FL-035-SER | FALSE | | 1 | 0 | 0 | 0 | 0 | 0 |
| USA_2016_FL-035-URI | TRUE | KY785440 | 1 | 1 | 0 | 0 | 0 | 0 |
| USA_2016_FL-036-SER | TRUE | KY785474 | 1 | 1 | 0 | 0 | 0 | 0 |
| USA_2016_FL-037-URI | FALSE | | 1 | 0 | 0 | 0 | 0 | 0 |
| USA_2016_FL-038-URI | TRUE | KY014326 | 1 | 1 | 0 | 0 | 1 | 0 |
| USA_2016_FL-039-URI | TRUE | KY014316 | 1 | 1 | 2 | 0 | 2 | 2 |
| USA_2016_FL-04-MOS | TRUE | KY014299 | 1 | 1 | 3 | 0 | 3 | 2 |
| USA_2016_FL-040-SER | FALSE | | 2 | 0 | 1 | 0 | 1 | 0 |
| USA_2016_FL-05-MOS | TRUE | KY785422 | 1 | 1 | 4 | 0 | 4 | 2 |
| USA_2016_FL-06-MOS | TRUE | KY785472 | 1 | 1 | 4 | 1 | 4 | 4 |
| USA_2016_FL-07-MOS | FALSE | | 1 | 0 | 4 | 0 | 4 | 0 |
| USA_2016_FL-08-MOS | TRUE | KY785468 | 1 | 1 | 2 | 0 | 2 | 2 |
| USA_2016_MA-WGS16-034-SER | TRUE | KY785459 | 1 | 1 | 0 | 0 | 1 | 0 |
| VEN_2016_FL-017-URI | FALSE | | 1 | 0 | 0 | 0 | 0 | 0 |

| | Pooled | Pooled | Pooled | Pooled | | | |
|---|---|---|---|---|---|---|---|
| | amplicon | amplicon | hybrid | hybrid | Prep | | |
| | sequencing | sequencing | capture | capture | method | Dependent | |
| | genome: | genome: | genome: | genome: | used for | variable used | |
| | unambig | genome | unambig | genome | genome | in regression | |
| Sample name | bases | length | bases | length | analysis | on metadata | Sample type |
| BLM_2016_MA-WGS16-006-SER | N/A | N/A | N/A | N/A | N/A | 0 | Serum |
| BRA_2016_FC-28209-CSF | N/A | N/A | N/A | N/A | N/A | 0 | Cerebrospinal |
| | | | | | | | flui [symbol] |
| BRA_2016_FC-546939-SER | N/A | N/A | N/A | N/A | N/A | 0 | Serum |
| BRA_2016_FC-547184-SER | N/A | N/A | N/A | N/A | N/A | 0 | Serum |
| BRA_2016_FC-547184-URI | N/A | N/A | N/A | N/A | N/A | 0 | Urine |
| BRA_2016_FC-547220-SER | N/A | N/A | N/A | N/A | N/A | 0 | Serum |
| BRA_2016_FC-547224-URI | N/A | N/A | N/A | N/A | N/A | 0 | Urine |
| BRA_2016_FC-547231-URI | N/A | N/A | N/A | N/A | N/A | 0 | Urine |
| BRA_2016_FC-5790-SER | 6996 | 10034 | N/A | N/A | amplicon sequencing | 6996 | Serum |
| BRA_2016_FC-5905-SER | 7467 | 10366 | N/A | N/A | amplicon sequencing | 7467 | Serum |
| BRA_2016_FC-6020-SOCO-SER | N/A | N/A | N/A | N/A | N/A | 0 | Serum |
| BRA_2016_FC-6095-SOCO-SER | N/A | N/A | N/A | N/A | N/A | 0 | Serum |
| BRA_2016_FC-6284-RNA-SER | N/A | N/A | N/A | N/A | N/A | 0 | Serum |
| BRA_2016_FC-6418-SER | 10227 | 10353 | 0 | 0 | amplicon sequencing | 6713.333333 | Serum |
| BRA_2016_FC-6696-SER | 9274 | 10356 | N/A | N/A | amplicon sequencing | 4032.666667 | Serum |
| BRA_2016_FC-6703-SER | 6606 | 10343 | N/A | N/A | amplicon sequencing | 3394 | Serum |
| BRA_2016_FC-6706-SER | 10030 | 10356 | 0 | 0 | amplicon sequencing | 6369.333333 | Serum |
| BRA_2016_FC-6863-SER | 10438 | 10455 | 2023 | 8584 | amplicon sequencing | 10169.5 | Serum |
| BRA_2016_FC-6864-URI | 10601 | 10602 | 8959 | 10654 | amplicon sequencing | 10476 | Urine |
| BRA_2016_FC-DQ105D1-PLA | N/A | N/A | N/A | N/A | N/A | 0 | Plasma EDTA |
| BRA_2016_FC-DQ107D1-URI | 10140 | 10351 | 4606 | 9567 | amplicon sequencing | 9815 | Urine |
| BRA_2016_FC-DQ109D1-PLA | N/A | N/A | N/A | N/A | N/A | 2331 | Plasma EDTA |
| BRA_2016_FC-DQ116D1-PLA | 9970 | 10455 | N/A | N/A | amplicon sequencing | 3432.333333 | Plasma EDTA |
| BRA_2016_FC-DQ119D1-URI | N/A | N/A | N/A | N/A | N/A | 1136 | Urine |
| BRA_2016_FC-DQ121D1-PLA | N/A | N/A | N/A | N/A | N/A | 0 | Plasma EDTA |
| BRA_2016_FC-DQ122D1-PLA | 9922 | 10455 | 1222 | 9119 | amplicon sequencing | 8343 | Plasma EDTA |
| BRA_2016_FC-DQ125D1-PLA | N/A | N/A | N/A | N/A | N/A | 0 | Plasma EDTA |
| BRA_2016_FC-DQ12D1-PLA | 4910 | 10032 | N/A | N/A | amplicon sequencing | 4909 | Plasma EDTA |
| BRA_2016_FC-DQ131D1-URI | 10602 | 10602 | 10788 | 10788 | hybrid capture | 10601 | Urine |
| BRA_2016_FC-DQ14D1-PLA | N/A | N/A | N/A | N/A | N/A | 0 | Plasma EDTA |
| BRA_2016_FC-DQ192D1-URI | 5061 | 7940 | N/A | N/A | amplicon sequencing | 1640.25 | Urine |
| BRA_2016_FC-DQ194D1-URI | N/A | N/A | N/A | N/A | N/A | 0 | Urine |
| BRA_2016_FC-DQ197D1-URI | N/A | N/A | N/A | N/A | N/A | 0 | Urine |
| BRA_2016_FC-DQ203D1-PLA | N/A | N/A | N/A | N/A | N/A | 0 | Plasma |
| BRA_2016_FC-DQ219D1-URI | N/A | N/A | N/A | N/A | N/A | 1013.666667 | Urine |
| BRA_2016_FC-DQ220D1-PLA | N/A | N/A | N/A | N/A | N/A | 0 | Plasma |
| BRA_2016_FC-DQ246D1-URI | N/A | N/A | N/A | N/A | N/A | 0 | Urine |
| BRA_2016_FC-DQ28D1-URI | 10596 | 10601 | 10752 | 10752 | hybrid capture | 6985 | Urine |
| BRA_2016_FC-DQ42D1-URI | 10599 | 10602 | 10751 | 10751 | hybrid capture | 10534.66667 | Urine |
| BRA_2016_FC-DQ47D1-PLA | 9937 | 10454 | N/A | N/A | amplicon sequencing | 9937 | Plasma EDTA |
| BRA_2016_FC-DQ49D1-PLA | 10249 | 10455 | 4060 | 10146 | amplicon sequencing | 9703 | Plasma EDTA |
| BRA_2016_FC-DQ52D1-PLA | N/A | N/A | N/A | N/A | N/A | 0 | Plasma EDTA |
| BRA_2016_FC-DQ58D1-PLA | 10227 | 10357 | 3895 | 8404 | amplicon sequencing | 9836 | Plasma EDTA |
| BRA_2016_FC-DQ5D1-URI | 9206 | 10366 | N/A | N/A | amplicon sequencing | 6967 | Urine |
| BRA_2016_FC-DQ60D1-PLA | 2722 | 6082 | N/A | N/A | amplicon sequencing | 2722 | Plasma EDTA |
| BRA_2016_FC-DQ60D1-URI | 10172 | 10354 | 6124 | 10454 | amplicon sequencing | 9286 | Urine |
| BRA_2016_FC-DQ62D1-PLA | 9628 | 10123 | N/A | N/A | amplicon sequencing | 7905 | Plasma EDTA |
| BRA_2016_FC-DQ62D1-URI | N/A | N/A | N/A | N/A | N/A | 0 | Urine |
| BRA_2016_FC-DQ62D2-PLA | N/A | N/A | N/A | N/A | N/A | 415 | Plasma EDTA |
| BRA_2016_FC-DQ62D2-URI | 3365 | 8781 | 10732 | 10732 | hybrid capture | 3365 | Urine |
| BRA_2016_FC-DQ68D1-URI | 3629 | 6697 | N/A | N/A | amplicon sequencing | 1677 | Urine |
| BRA_2016_FC-DQ75D1-PLA | 2718 | 8846 | N/A | N/A | amplicon sequencing | 963.3333333 | Plasma EDTA |
| BRA_2016_FC-DQ75D1-URI | 10162 | 10355 | 6382 | 9727 | amplicon sequencing | 9556 | Urine |
| BRA_2016_FC-DQ77D1-URI | N/A | N/A | N/A | N/A | N/A | 226.5 | Urine |
| COL_2016_SU-1155A-SER | N/A | N/A | N/A | N/A | N/A | 1793 | Serum |
| COL_2016_SU-1194A-SER | N/A | N/A | N/A | N/A | N/A | 2029 | Serum |
| COL_2016_SU-1638A-SER | N/A | N/A | N/A | N/A | N/A | 446 | Serum |
| COL_2016_SU-1810A-SER | 5366 | 10454 | N/A | N/A | amplicon sequencing | 5365 | Serum |
| COL_2016_SU-1856A-SER | 10351 | 10354 | 5580 | 9734 | amplicon sequencing | 10351 | Serum |
| COL_2016_SU-1857A-SER | N/A | N/A | N/A | N/A | N/A | 930 | Serum |
| COL_2016_SU-1898A-SER | N/A | N/A | N/A | N/A | N/A | 0 | Serum |
| COL_2016_SU-1938A-SER | N/A | N/A | N/A | N/A | N/A | 1802 | Serum |
| COL_2016_SU-2293A-SER | 5226 | 9396 | N/A | N/A | amplicon sequencing | 5219 | Serum |
| COL_2016_SU-2514A-SER | N/A | N/A | N/A | N/A | N/A | 0 | Serum |
| COL_2016_SU-2636A-SER | N/A | N/A | N/A | N/A | N/A | 0 | Serum |
| COL_2016_SU-2686A-SER | N/A | N/A | N/A | N/A | N/A | 0 | Serum |
| COL_2016_SU-2724A-SER | 10594 | 10597 | 5727 | 10192 | amplicon sequencing | 10594 | Serum |
| COL_2016_SU-2836A-SER | N/A | N/A | N/A | N/A | N/A | 0 | Serum |
| COL_2016_SU-3117A-SER | N/A | N/A | N/A | N/A | N/A | 0 | Serum |
| COL_2016_SU-3311A-SER | N/A | N/A | N/A | N/A | N/A | 0 | Serum |
| COL_2016_SU-3315A-SER | N/A | N/A | N/A | N/A | N/A | 0 | Serum |
| COL_2016_SU-3316A-SER | N/A | N/A | N/A | N/A | N/A | 0 | Serum |
| COL_2016_SU-3351A-SER | N/A | N/A | N/A | N/A | N/A | 468 | Serum |
| COL_2016_SU-3407A-SER | N/A | N/A | N/A | N/A | N/A | 0 | Serum |
| CUB_2016_FL-011-URI | N/A | N/A | N/A | N/A | N/A | 0 | Urine |
| DOM_2016_BB-0054-SER | N/A | N/A | N/A | N/A | N/A | 0 | Serum |
| DOM_2016_BB-0054-URI | N/A | N/A | N/A | N/A | N/A | 1649 | Urine |
| DOM_2016_BB-0059-SER | 9408 | 10035 | N/A | N/A | amplicon sequencing | 7723.5 | Serum |
| DOM_2016_BB-0059-URI | N/A | N/A | N/A | N/A | N/A | 751 | Urine |
| DOM_2016_BB-0071-SER | 9409 | 10455 | 0 | 0 | amplicon sequencing | 7956 | Serum |
| DOM_2016_BB-0071-URI | 5555 | 10364 | N/A | N/A | amplicon sequencing | 5555 | Urine |
| DOM_2016_BB-0076-SER | 10364 | 10366 | 1344 | 9109 | amplicon sequencing | 9355 | Serum |
| DOM_2016_BB-0076-URI | 10158 | 10367 | 997 | 9856 | amplicon sequencing | 8472.333333 | Urine |
| DOM_2016_BB-0085-SER | 4003 | 8806 | N/A | N/A | amplicon sequencing | 4002 | Serum |
| DOM_2016_BB-0085-URI | 10160 | 10453 | N/A | N/A | amplicon sequencing | 9907.5 | Urine |
| DOM_2016_BB-0091-SER | 10356 | 10366 | N/A | N/A | amplicon sequencing | 10255 | Serum |
| DOM_2016_BB-0115-SER | 10602 | 10602 | 10782 | 10782 | hybrid capture | 10468.66667 | Serum |
| DOM_2016_BB-0115-URI | 10426 | 10456 | N/A | N/A | amplicon sequencing | 9447.666667 | Urine |
| DOM_2016_BB-0127-SER | 10599 | 10599 | 10739 | 10739 | hybrid capture | 10599 | Serum |
| DOM_2016_BB-0180-SER | 10601 | 10602 | 10783 | 10783 | hybrid capture | 10488.5 | Serum |
| DOM_2016_BB-0180-URI | 10436 | 10456 | 0 | 0 | amplicon sequencing | 9550.333333 | Urine |
| DOM_2016_BB-0183-SER | 10621 | 10621 | N/A | N/A | amplicon sequencing | 10248.5 | Serum |
| DOM_2016_BB-0183-URI | N/A | N/A | N/A | N/A | N/A | 0 | Urine |
| DOM_2016_BB-0184-URI | N/A | N/A | N/A | N/A | N/A | 471 | Urine |
| DOM_2016_BB-0208-SER | 10598 | 10600 | 10753 | 10753 | hybrid capture | 10598 | Serum |
| DOM_2016_BB-0216-SER | 10577 | 10581 | 9743 | 10595 | amplicon sequencing | 10577 | Serum |
| DOM_2016_BB-0269-SER | 10611 | 10615 | 9124 | 10342 | amplicon sequencing | 10611 | Serum |
| DOM_2016_BB-0369-SER | N/A | N/A | N/A | N/A | N/A | 0 | Serum |
| DOM_2016_BB-0428-PLA | 4607 | 9732 | N/A | N/A | amplicon sequencing | 4607 | Plasma |
| DOM_2016_BB-0433-SER | 10365 | 10365 | 722 | 3591 | amplicon sequencing | 10185 | Serum |
| DOM_2016_BB-0436-PLA | 9565 | 10357 | 10675 | 10681 | hybrid capture | 9565 | Plasma |
| DOM_2016_BB-0439-SER | N/A | N/A | N/A | N/A | N/A | 0 | Serum |
| DOM_2016_BB-0445-SER | N/A | N/A | N/A | N/A | N/A | 0 | Serum |
| DOM_2016_BB-0448-SER | N/A | N/A | N/A | N/A | N/A | 0 | Serum |
| DOM_2016_FL-002-URI | N/A | N/A | N/A | N/A | N/A | 0 | Urine |
| DOM_2016_FL-014-URI | N/A | N/A | N/A | N/A | N/A | 0 | Urine |
| DOM_2016_MA-WGS16-007-SER | 10151 | 10352 | 6416 | 9838 | amplicon sequencing | 10151 | Serum |
| DOM_2016_MA-WGS16-009-SER | 2856 | 6160 | N/A | N/A | amplicon sequencing | 2856 | Serum |
| DOM_2016_MA-WGS16-010-SER | N/A | N/A | N/A | N/A | N/A | 0 | Serum |
| DOM_2016_MA-WGS16-011-SER | 10379 | 10596 | 0 | 0 | amplicon sequencing | 10379 | Serum |
| DOM_2016_MA-WGS16-013-SER | 7176 | 10071 | N/A | N/A | amplicon sequencing | 7175 | Serum |
| DOM_2016_MA-WGS16-014-SER | 9698 | 10361 | 2604 | 9659 | amplicon sequencing | 9698 | Serum |
| DOM_2016_MA-WGS16-020-SER | 3037 | 9384 | N/A | N/A | amplicon sequencing | 3037 | Serum |
| DOM_2016_MA-WGS16-021-SER | N/A | N/A | N/A | N/A | N/A | 0 | Serum |
| DOM_2016_MA-WGS16-024-SER | 10564 | 10596 | 10180 | 10374 | amplicon sequencing | 10564 | Serum |
| DOM_2016_MA-WGS16-028-SER | N/A | N/A | N/A | N/A | N/A | 0 | Serum |
| DOM_2016_MA-WGS16-031-SER | 2639 | 9442 | N/A | N/A | amplicon sequencing | 2641 | Serum |
| DOM_2016_MA-WGS16-035-SER | N/A | N/A | N/A | N/A | N/A | 0 | Serum |
| DOM_2016_MA-WGS16-036-SER | 5813 | 10359 | N/A | N/A | amplicon sequencing | 5813 | Serum |
| DOM_2016_MA-WGS16-040-SER | 8308 | 10345 | N/A | N/A | amplicon sequencing | 8309 | Serum |
| GTM_2016_MA-WGS16-026-SER | N/A | N/A | N/A | N/A | N/A | 0 | Serum |
| GUY_2016_MA-WGS16-012-SER | N/A | N/A | N/A | N/A | N/A | 0 | Serum |
| HND_2016_HU-ME131-PLA | 9498 | 10347 | N/A | N/A | amplicon sequencing | 9498 | Plasma |
| HND_2016_HU-ME136-PLA | 5856 | 10361 | N/A | N/A | amplicon sequencing | 5856 | Plasma |
| HND_2016_HU-ME137-PLA | 4471 | 10449 | N/A | N/A | amplicon sequencing | 4470 | Plasma |
| HND_2016_HU-ME147-SER | 10409 | 10428 | 5593 | 9518 | amplicon sequencing | 9555.5 | Serum |
| HND_2016_HU-ME149-PLA | N/A | N/A | N/A | N/A | N/A | 0 | Plasma |
| HND_2016_HU-ME152-SER | 10365 | 10366 | 7680 | 9382 | amplicon sequencing | 10279 | Serum |
| HND_2016_HU-ME156-SER | 10260 | 10455 | 1165 | 3062 | amplicon sequencing | 9636.5 | Serum |
| HND_2016_HU-ME167-PLA | 10596 | 10597 | 10327 | 10619 | hybrid capture | 10596 | Plasma |
| HND_2016_HU-ME171-PLA | 10433 | 10453 | 0 | 0 | amplicon sequencing | 9868 | Plasma |
| HND_2016_HU-ME172-PLA | 6431 | 10013 | N/A | N/A | amplicon sequencing | 6431 | Plasma |
| HND_2016_HU-ME178-PLA | 10437 | 10455 | 5939 | 9653 | amplicon sequencing | 10084 | Plasma |
| HND_2016_HU-ME180-PLA | 8531 | 10034 | N/A | N/A | amplicon sequencing | 8531 | Plasma |
| HND_2016_HU-ME33-PLA | 5644 | 10250 | N/A | N/A | amplicon sequencing | 5648 | Plasma |
| HND_2016_HU-ME38-PLA | 10364 | 10367 | 10677 | 10682 | hybrid capture | 10364 | Plasma |
| HND_2016_HU-ME42-SER | 10070 | 10325 | 10655 | 10676 | hybrid capture | 10070 | Serum |
| HND_2016_HU-ME50-PLA | 7862 | 9265 | N/A | N/A | amplicon sequencing | 7862 | Plasma |
| HND_2016_HU-ME58-PLA | 10365 | 10366 | 10779 | 10779 | hybrid capture | 10365 | Plasma |
| HND_2016_HU-ME59-PLA | 10362 | 10365 | 7718 | 10191 | amplicon sequencing | 9941 | Plasma |
| HND_2016_HU-SZ28-SER | N/A | N/A | N/A | N/A | N/A | 0 | Serum |
| HND_2016_HU-SZ76-SER | 5535 | 10183 | N/A | N/A | amplicon sequencing | 5535 | Serum |
| HTI_2016_FL-018-URI | N/A | N/A | N/A | N/A | N/A | 0 | Urine |
| HTI_2016_MA-WGS16-002-SER | N/A | N/A | N/A | N/A | N/A | 0 | Serum |
| HTI_2016_MA-WGS16-003-SER | N/A | N/A | N/A | N/A | N/A | 0 | Serum |
| HTI_2016_MA-WGS16-022-SER | 3961 | 7005 | N/A | N/A | amplicon sequencing | 3961 | Serum |
| JAM_2016_FL-006-SER | N/A | N/A | N/A | N/A | N/A | 0 | Serum |
| JAM_2016_FL-006-URI | N/A | N/A | N/A | N/A | N/A | 0 | Urine |
| JAM_2016_FL-007-URI | N/A | N/A | N/A | N/A | N/A | 0 | Urine |
| JAM_2016_FL-012-URI | N/A | N/A | N/A | N/A | N/A | 1080 | Urine |
| JAM_2016_FL-015-URI | N/A | N/A | N/A | N/A | N/A | 0 | Urine |
| JAM_2016_MA-WGS16-018-SER | N/A | N/A | N/A | N/A | N/A | 492 | Serum |
| JAM_2016_MA-WGS16-025-SER | 9371 | 10360 | 0 | 0 | amplicon sequencing | 9371 | Serum |
| JAM_2016_MA-WGS16-037-SER | N/A | N/A | N/A | N/A | N/A | 0 | Serum |
| JAM_2016_MA-WGS16-038-SER | 4807 | 8499 | N/A | N/A | amplicon sequencing | 4815 | Serum |
| JAM_2016_MA-WGS16-039-SER | 8602 | 10364 | 3343 | 9284 | amplicon sequencing | 8601 | Serum |
| JAM_2016_MA-WGS16-041-SER | 5379 | 10367 | N/A | N/A | amplicon sequencing | 5379 | Serum |
| JAM_2016_WI-JM2-SER | N/A | N/A | N/A | N/A | N/A | 0 | Serum |
| JAM_2016_WI-JM3A-SER | N/A | N/A | N/A | N/A | N/A | 465 | Serum |
| JAM_2016_WI-JM3B-URI | N/A | N/A | N/A | N/A | N/A | 0 | Urine |
| JAM_2016_WI-JM4-SER | N/A | N/A | N/A | N/A | N/A | 0 | Serum |
| JAM_2016_WI-JM5-SER | N/A | N/A | N/A | N/A | N/A | 0 | Serum |
| JAM_2016_WI-JM6-SER | 10357 | 10599 | 678 | 6466 | amplicon sequencing | 10357 | Serum |
| JAM_2016_WI-JM7A-SER | N/A | N/A | N/A | N/A | N/A | 0 | Serum |
| JAM_2016_WI-JM7B-URI | N/A | N/A | N/A | N/A | N/A | 0 | Urine |
| JAM_2016_WI-JM8-SER | N/A | N/A | N/A | N/A | N/A | 0 | Serum |
| MEX_2016_MA-WGS16-030-SER | N/A | N/A | N/A | N/A | N/A | 0 | Serum |
| MTQ_2016_FL-001-SAL | 8191 | 10345 | N/A | N/A | amplicon sequencing | 8192 | Saliva |
| MTQ_2016_FL-001-SER | N/A | N/A | N/A | N/A | N/A | 0 | Serum |
| MTQ_2016_FL-001-URI | N/A | N/A | N/A | N/A | N/A | 0 | Urine |
| PRI_2016_FL-004-SER | N/A | N/A | N/A | N/A | N/A | 0 | Serum |
| PRI_2016_FL-004-URI | N/A | N/A | N/A | N/A | N/A | 0 | Urine |
| PRI_2016_FL-005-SER | N/A | N/A | N/A | N/A | N/A | 450 | Serum |
| PRI_2016_FL-005-URI | N/A | N/A | N/A | N/A | N/A | 461 | Urine |
| PRI_2016_FL-008-URI | N/A | N/A | N/A | N/A | N/A | 0 | Urine |
| PRI_2016_FL-009-URI | N/A | N/A | N/A | N/A | N/A | 0 | Urine |
| PRI_2016_FL-013-URI | N/A | N/A | N/A | N/A | N/A | 2411 | Urine |
| PRI_2016_FL-016-URI | N/A | N/A | N/A | N/A | N/A | 0 | Urine |
| PRI_2016_MA-WGS16-004-SER | 10439 | 10453 | 5540 | 9740 | amplicon sequencing | 10439 | Serum |
| PRI_2016_MA-WGS16-005-SER | 7293 | 10058 | N/A | N/A | amplicon sequencing | 7293 | Serum |
| PRI_2016_MA-WGS16-015-SER | N/A | N/A | N/A | N/A | N/A | 0 | Serum |
| PRI_2016_MA-WGS16-016-SER | 9816 | 10456 | 0 | 0 | amplicon sequencing | 9816 | Serum |
| PRI_2016_MA-WGS16-017-SER | N/A | N/A | N/A | N/A | N/A | 0 | Serum |
| PRI_2016_MA-WGS16-019-SER | N/A | N/A | N/A | N/A | N/A | 0 | Serum |
| PRI_2016_MA-WGS16-032-SER | N/A | N/A | N/A | N/A | N/A | 0 | Serum |
| SLV_2016_MA-WGS16-001-SER | N/A | N/A | N/A | N/A | N/A | 0 | Serum |
| TTO_2016_FL-003-URI | N/A | N/A | N/A | N/A | N/A | 674 | Urine |
| TTO_2016_MA-WGS16-027-SER | N/A | N/A | N/A | N/A | N/A | 0 | Serum |
| UNK_2016_MA-WGS16-008-SER | N/A | N/A | N/A | N/A | N/A | 887 | Serum |
| UNK_2016_MA-WGS16-023-SER | N/A | N/A | N/A | N/A | N/A | 0 | Serum |
| UNK_2016_MA-WGS16-029-SER | 6082 | 8515 | N/A | N/A | amplicon sequencing | 6082 | Serum |
| UNK_2016_MA-WGS16-033-SER | N/A | N/A | N/A | N/A | N/A | 414 | Serum |
| USA_2016_FL-01-MOS | 10593 | 10599 | 10786 | 10786 | hybrid capture | 10593 | Mosquito |
| USA_2016_FL-010-SER | N/A | N/A | N/A | N/A | N/A | 0 | Serum |
| USA_2016_FL-010-URI | 10375 | 10388 | 10778 | 10778 | hybrid capture | 10375 | Urine |
| USA_2016_FL-019-URI | 3093 | 7926 | N/A | N/A | amplicon sequencing | 3093 | Urine |
| USA_2016_FL-02-MOS | 10439 | 10454 | 10783 | 10783 | hybrid capture | 10439 | Mosquito |
| USA_2016_FL-020-URI | N/A | N/A | N/A | N/A | N/A | 816 | Urine |
| USA_2016_FL-021-URI | 10431 | 10501 | 2907 | 9781 | amplicon sequencing | 10278.5 | Urine |
| USA_2016_FL-022-URI | 9222 | 10599 | N/A | N/A | amplicon sequencing | 9222 | Urine |
| USA_2016_FL-023-URI | N/A | N/A | N/A | N/A | N/A | 0 | Urine |
| USA_2016_FL-023-WBL | N/A | N/A | N/A | N/A | N/A | 819 | Whole blood |
| USA_2016_FL-025-URI | N/A | N/A | N/A | N/A | N/A | 0 | Urine |
| USA_2016_FL-026-URI | N/A | N/A | N/A | N/A | N/A | 0 | Urine |
| USA_2016_FL-027-URI | N/A | N/A | N/A | N/A | N/A | 1118 | Urine |
| USA_2016_FL-028-SER | 8360 | 10033 | N/A | N/A | amplicon sequencing | 8360 | Serum |
| USA_2016_FL-028-URI | N/A | N/A | N/A | N/A | N/A | 1187.5 | Urine |
| USA_2016_FL-029-URI | 7048 | 10349 | N/A | N/A | amplicon sequencing | 4078 | Urine |
| USA_2016_FL-03-MOS | 10594 | 10594 | 10779 | 10779 | hybrid capture | 10594 | Mosquito |
| USA_2016_FL-030-URI | 10601 | 10601 | 1583 | 5933 | amplicon sequencing | 9899.5 | Urine |
| USA_2016_FL-031-URI | N/A | N/A | N/A | N/A | N/A | 1951 | Urine |
| USA_2016_FL-032-URI | 9863 | 10364 | 6230 | 10291 | amplicon sequencing | 9863 | Urine |
| USA_2016_FL-033-URI | N/A | N/A | N/A | N/A | N/A | 903 | Urine |
| USA_2016_FL-034-SER | N/A | N/A | N/A | N/A | N/A | 1668 | Serum |
| USA_2016_FL-034-URI | N/A | N/A | N/A | N/A | N/A | 428 | Urine |
| USA_2016_FL-035-SER | N/A | N/A | N/A | N/A | N/A | 2069 | Serum |
| USA_2016_FL-035-URI | 2846 | 5818 | N/A | N/A | amplicon sequencing | 2846 | Urine |
| USA_2016_FL-036-SER | 7645 | 10597 | N/A | N/A | amplicon sequencing | 7645 | Serum |
| USA_2016_FL-037-URI | N/A | N/A | N/A | N/A | N/A | 1962 | Urine |
| USA_2016_FL-038-URI | 8763 | 10365 | 529 | 6452 | amplicon sequencing | 8763 | Urine |
| USA_2016_FL-039-URI | 10597 | 10600 | 10728 | 10729 | hybrid capture | 10597 | Urine |
| USA_2016_FL-04-MOS | 10433 | 10455 | 10724 | 10727 | hybrid capture | 10433 | Mosquito |
| USA_2016_FL-040-SER | N/A | N/A | N/A | N/A | N/A | 340 | Serum |
| USA_2016_FL-05-MOS | 10439 | 10455 | 8487 | 10574 | amplicon sequencing | 10439 | Mosquito |
| USA_2016_FL-06-MOS | 10586 | 10594 | 10583 | 10758 | hybrid capture | 10586 | Mosquito |
| USA_2016_FL-07-MOS | N/A | N/A | N/A | N/A | N/A | 0 | Mosquito |
| USA_2016_FL-08-MOS | 10360 | 10365 | 9851 | 10450 | amplicon sequencing | 10360 | Mosquito |
| USA_2016_MA-WGS16-034-SER | 9905 | 10341 | 0 | 0 | amplicon sequencing | 9906 | Serum |
| VEN_2016_FL-017-URI | N/A | N/A | N/A | N/A | N/A | 0 | Urine |

[symbol]

indicates data missing or illegible when filed

TABLE 8
Table listing observed nonsynonymous SNPs across data used for SNP analysis.

| | | | Allele frequency | | | | Impact on ZIKV proteins | | | | |
|---|---|---|---|---|---|---|---|---|---|---|---|
| Position | Allele | | within outbreak | | Number of alleles | | Codon | | | Amino acid | |
| (KX197192.1) | Ancesteral | Derived | Ancesteral | Derived | Minor | Total | position | Degeneracy | Codon change | change | Protein |
| 138 | T | C | 0.99329 | 0.00671 | 1 | 149 | 11 | 1 | TTC −> CTC | F −> L | capsid |
| 186 | T | C | 0.99333 | 0.00667 | 1 | 150 | 27 | 1 | TTT −> CTT | F −> L | capsid |
| 346 | T | C | 0.97297 | 0.02703 | 4 | 148 | 80 | 1 | ATA −> ACA | I −> T | capsid |
| 350 | A | G | 0.99338 | 0.00662 | 1 | 151 | 81 | 3 | ATA −> ATG | I −> M | capsid |
| 420 | G | A | 0.99401 | 0.00599 | 1 | 167 | 105 | 1 | GGC −> AGC | G −> S | capsid |
| 428 | T | A | 0.86145 | 0.12651 | 21 | 166 | 107 | 2 | GAT −> GAA | D −> E | capsid |
| 439 | G | A | 0.99401 | 0.00599 | 1 | 167 | 111 | 1 | GGA −> GAA | G −> E | capsid |
| 444 | G | A | 0.98802 | 0.01198 | 2 | 167 | 113 | 1 | GTT −> ATT | V −> I | capsid |
| 524 | C | A | 0.99363 | 0.00637 | 1 | 157 | 17 | 2 | AAC −> AAA | N −> K | propeptide |
| 604 | T | C | 0.99367 | 0.00633 | 1 | 158 | 44 | 1 | ATG −> ACG | M −> T | propeptide |
| 616 | C | T | 0.99363 | 0.00637 | 1 | 157 | 48 | 1 | ACC −> ATC | T −> I | propeptide |
| 666 | G | A | 0.99351 | 0.00649 | 1 | 154 | 65 | 1 | GTC −> ATC | V −> I | propeptide |
| 709 | G | T | 0.9939 | 0.0061 | 1 | 164 | 79 | 1 | GGA −> GTA | G −> V | propeptide |
| 739 | G | A | 0.99387 | 0.00613 | 1 | 163 | 89 | 1 | CGG −> CAG | R −> Q | propeptide |
| 792 | A | G | 0.9875 | 0.0125 | 2 | 160 | 14 | 1 | ACG −> GCG | T −> A | membrane |
| 810 | T | A | 0.99301 | 0.00699 | 1 | 143 | 20 | 2 | TTG −> ATG | L −> M | membrane |
| 1044 | G | A | 0.97468 | 0.02532 | 4 | 158 | 23 | 1 | GTT −> ATT | V −> I | envelope |
| 1116 | A | T | 0.9932 | 0.0068 | 1 | 147 | 47 | 1 | ACA −> TCA | T −> S | envelope |
| 1143 | G | A | 0.98639 | 0.01361 | 2 | 147 | 56 | 2 | GTA −> ATA | V −> I | envelope |
| 1152 | T | C | 0.9932 | 0.0068 | 1 | 147 | 59 | 1 | TAC −> CAC | Y −> H | envelope |
| 1167 | T | A | 0.99329 | 0.00671 | 1 | 149 | 64 | 1 | TCA −> ACA | S −> T | envelope |
| 1180 | T | C | 0.99329 | 0.00671 | 1 | 149 | 68 | 1 | ATG −> ACG | M −> T | envelope |
| 1181 | G | A | 0.99329 | 0.00671 | 1 | 149 | 68 | 3 | ATG −> ATA | M −> I | envelope |
| 1183 | C | T | 0.99329 | 0.00671 | 1 | 149 | 69 | 1 | GCT −> GTT | A −> V | envelope |
| 1360 | A | G | 0.99351 | 0.00649 | 1 | 154 | 128 | 1 | AAG −> AGG | K −> R | envelope |
| 1387 | A | T | 0.99359 | 0.00641 | 1 | 156 | 137 | 1 | TAC −> TTC | Y −> F | envelope |
| 1521 | G | A | 0.99286 | 0.00714 | 1 | 140 | 182 | 1 | GGT −> AGT | G −> S | envelope |
| 1561 | G | C | 0.98701 | 0.01299 | 2 | 154 | 195 | 1 | GGC −> GCC | G −> A | envelope |
| 1714 | A | G | 0.99375 | 0.00625 | 1 | 160 | 246 | 1 | AAG −> AGG | K −> R | envelope |
| 1718 | C | A | 0.99363 | 0.00637 | 1 | 157 | 247 | 2 | GAC −> GAA | D −> E | envelope |
| 1741 | T | C | 0.99359 | 0.00641 | 1 | 156 | 255 | 1 | GTC −> GCC | V −> A | envelope |
| 1747 | T | C | 0.99367 | 0.00633 | 1 | 158 | 257 | 1 | GTT −> GCT | V −> A | envelope |
| 1756 | G | C | 0.99363 | 0.00637 | 1 | 157 | 260 | 1 | AGT −> ACT | S −> T | envelope |
| 1816 | C | T | 0.99359 | 0.00641 | 1 | 156 | 280 | 1 | GCA −> GTA | A −> V | envelope |
| 1902 | A | T | 0.99394 | 0.00606 | 1 | 165 | 309 | 1 | ACC −> TCC | T −> S | envelope |
| 1903 | C | A | 0.98182 | 0.01818 | 3 | 165 | 309 | 1 | ACC −> AAC | T −> N | envelope |
| 1965 | G | T | 0.99387 | 0.00613 | 1 | 163 | 330 | 2 | GTA −> TTA | V −> L | envelope |
| 1980 | A | G | 0.99363 | 0.00637 | 1 | 157 | 335 | 1 | ACA −> GCA | T −> A | envelope |
| 2023 | T | C | 0.99359 | 0.00641 | 1 | 156 | 349 | 1 | ATG −> ACG | M −> T | envelope |
| 2028 | A | T | 0.99359 | 0.00641 | 1 | 156 | 351 | 1 | ACT −> TCT | T −> S | envelope |
| 2079 | A | G | 0.99371 | 0.00629 | 1 | 159 | 368 | 1 | AGC −> GGC | S −> G | envelope |
| 2083 | C | T | 0.99371 | 0.00629 | 1 | 159 | 369 | 1 | ACT −> ATT | T −> I | envelope |
| 2169 | C | T | 0.99387 | 0.00613 | 1 | 163 | 398 | 1 | CAC −> TAC | H −> Y | envelope |
| 2319 | A | G | 0.9932 | 0.0068 | 1 | 147 | 448 | 1 | ATT −> GTT | I −> V | envelope |
| 2412 | A | G | 0.99324 | 0.00676 | 1 | 148 | 479 | 1 | ACA −> GCA | T −> A | envelope |
| 2449 | T | C | 0.99324 | 0.00676 | 1 | 148 | 491 | 2 | TTA −> TCA | L −> S | envelope |
| 2511 | T | C | 0.98734 | 0.01266 | 2 | 158 | 8 | 1 | TTC −> CTC | F −> L | NS1 |
| 2527 | C | T | 0.99367 | 0.00633 | 1 | 158 | 13 | 1 | ACG −> ATG | T −> M | NS1 |
| 2678 | G | A | 0.9931 | 0.0069 | 1 | 145 | 63 | 3 | ATG −> ATA | M −> I | NS1 |
| 2781 | T | C | 0.99383 | 0.00617 | 1 | 162 | 98 | 2 | TGG −> CGG | W −> R | NS1 |
| 2788 | G | C | 0.89506 | 0.10494 | 17 | 162 | 100 | 1 | GGT −> GCT | G −> A | NS1 |
| 2795 | G | T | 0.99383 | 0.00617 | 1 | 162 | 102 | 2 | CAG −> CAT | Q −> H | NS1 |
| 2853 | C | T | 0.03704 | 0.96296 | 6 | 162 | 122 | 1 | CAC −> TAC | H −> Y | NS1 |
| 2923 | T | C | 0.99333 | 0.00667 | 1 | 150 | 145 | 1 | CTC −> CCC | L −> P | NS1 |
| 2925 | A | G | 0.99333 | 0.00667 | 1 | 150 | 146 | 1 | AAA −> GAA | K −> E | NS1 |
| 2944 | G | A | 0.99342 | 0.00658 | 1 | 152 | 152 | 1 | AGC −> AAC | S −> N | NS1 |
| 2946 | T | C | 0.98684 | 0.01316 | 2 | 152 | 153 | 1 | TTT −> CTT | F −> L | NS1 |
| 3034 | C | T | 0.99333 | 0.00667 | 1 | 150 | 182 | 1 | GCC −> GTC | A −> V | NS1 |
| 3052 | T | C | 0.99379 | 0.00621 | 1 | 161 | 188 | 1 | GTT −> GCT | V −> A | NS1 |
| 3058 | G | A | 0.99375 | 0.00625 | 1 | 160 | 190 | 1 | GGA −> GAA | G −> E | NS1 |
| 3061 | A | G | 0.98125 | 0.01875 | 3 | 160 | 191 | 1 | AAG −> AGG | K −> R | NS1 |
| 3070 | T | C | 0.975 | 0.025 | 4 | 160 | 194 | 1 | GTA −> GCA | V −> A | NS1 |
| 3112 | A | G | 0.99379 | 0.00621 | 1 | 161 | 208 | 1 | GAC −> GGC | D −> G | NS1 |
| 3186 | A | G | 0.99324 | 0.00676 | 1 | 148 | 233 | 1 | ACA −> GCA | T −> A | NS1 |
| 3214 | T | C | 0.99324 | 0.00676 | 1 | 148 | 242 | 1 | ATC −> ACC | I −> T | NS1 |
| 3223 | A | G | 0.9932 | 0.0068 | 1 | 147 | 245 | 1 | AAG −> AGG | K −> R | NS1 |
| 3246 | C | T | 0.9932 | 0.0068 | 1 | 147 | 253 | 1 | CAT −> TAT | H −> Y | NS1 |
| 3315 | C | T | 0.99333 | 0.00667 | 1 | 150 | 276 | 2 | CGG −> TGG | R −> W | NS1 |
| 3459 | C | T | 0.92727 | 0.07273 | 12 | 165 | 324 | 2 | CGG −> TGG | R −> W | NS1 |
| 3514 | A | G | 0.99367 | 0.00633 | 1 | 158 | 342 | 1 | GAA −> GGA | E −> G | NS1 |
| 3534 | A | G | 0.64557 | 0.35443 | 56 | 158 | 349 | 2 | ATG −> GTG | M −> V | NS1 |
| 3535 | T | C | 0.99367 | 0.00633 | 1 | 158 | 349 | 1 | ATG −> ACG | M −> T | NS1 |
| 3601 | T | C | 0.99367 | 0.00633 | 1 | 158 | 19 | 1 | ATG −> ACG | M −> T | NS2A |
| 3631 | C | T | 0.99363 | 0.00637 | 1 | 157 | 29 | 1 | ACC −> ATC | T −> I | NS2A |
| 3658 | T | C | 0.99363 | 0.00637 | 1 | 157 | 38 | 1 | ATG −> ACG | M −> T | NS2A |
| 3700 | G | A | 0.99398 | 0.00602 | 1 | 166 | 52 | 1 | AGT −> AAT | S −> N | NS2A |
| 3718 | C | T | 0.99398 | 0.00602 | 1 | 166 | 58 | 1 | GCA −> GTA | A −> V | NS2A |
| 3771 | C | T | 0.99394 | 0.00606 | 1 | 165 | 76 | 1 | CAT −> TAT | H −> Y | NS2A |
| 3784 | T | C | 0.99359 | 0.00641 | 1 | 156 | 80 | 1 | ATA −> ACA | I −> T | NS2A |
| 3882 | C | T | 0.99359 | 0.00641 | 1 | 156 | 113 | 1 | CTT −> TTT | L −> F | NS2A |
| 3925 | T | C | 0.98593 | 0.01307 | 2 | 153 | 127 | 1 | GTT −> GCT | V −> A | NS2A |
| 3993 | A | G | 0.99367 | 0.00633 | 1 | 158 | 150 | 1 | ATC −> GTC | I −> V | NS2A |
| 3999 | T | A | 0.96203 | 0.00633 | 1 | 158 | 152 | 2 | TTG −> ATG | L −> M | NS2A |
| 4029 | G | A | 0.99371 | 0.00629 | 1 | 159 | 162 | 1 | GCC −> ACC | A −> T | NS2A |
| 4030 | C | T | 0.98742 | 0.01258 | 2 | 159 | 162 | 1 | GCC −> GTC | A −> V | NS2A |
| 4047 | G | A | 0.99371 | 0.00629 | 1 | 159 | 168 | 2 | GTG −> ATG | V −> M | NS2A |
| 4113 | A | G | 0.99286 | 0.00714 | 1 | 140 | 190 | 1 | AGT −> GGT | S −> G | NS2A |
| 4213 | G | A | 0.99286 | 0.00714 | 1 | 140 | 223 | 1 | AGT −> AAT | S −> N | NS2A |
| 4287 | G | A | 0.93421 | 0.06579 | 10 | 152 | 22 | 1 | GCC −> ACC | A −> T | NS2B |
| 4319 | G | A | 0.99346 | 0.00654 | 1 | 153 | 32 | 3 | ATG −> ATA | M −> I | NS2B |
| 4363 | A | G | 0.99359 | 0.00641 | 1 | 156 | 47 | 1 | AAG −> AGG | K −> R | NS2B |
| 4536 | A | G | 0.99225 | 0.00775 | 1 | 129 | 105 | 1 | ACC −> GCC | T −> A | NS2B |
| 4651 | T | C | 0.9875 | 0.0125 | 2 | 160 | 13 | 1 | GTA −> GCA | V −> A | NS3 |
| 4663 | A | G | 0.98734 | 0.01266 | 2 | 158 | 17 | 1 | GAG −> GGG | E −> G | NS3 |
| 4956 | A | G | 0.98667 | 0.01333 | 2 | 150 | 115 | 2 | ATA −> GTA | I −> V | NS3 |
| 4957 | T | C | 0.99333 | 0.00667 | 1 | 150 | 115 | 1 | ATA −> ACA | I −> T | NS3 |
| 4972 | A | G | 0.97857 | 0.02143 | 3 | 140 | 120 | 1 | GAT −> GGT | D −> G | NS3 |
| 4989 | G | A | 0.99123 | 0.00877 | 1 | 114 | 126 | 1 | GTT −> ATT | V −> I | NS3 |
| 5038 | A | G | 0.99123 | 0.00877 | 1 | 114 | 142 | 1 | AAG −> AGG | K −> R | NS3 |
| 5232 | G | A | 0.99324 | 0.00676 | 1 | 148 | 207 | 1 | GAA −> AAA | E −> K | NS3 |
| 5614 | T | C | 0.98592 | 0.01408 | 2 | 142 | 334 | 1 | ATG −> ACG | M −> T | NS3 |
| 5671 | C | T | 0.9931 | 0.0069 | 1 | 145 | 353 | 1 | ACG −> ATG | T −> M | NS3 |
| 5676 | C | T | 0.97279 | 0.02721 | 4 | 147 | 355 | 1 | CAT −> TAT | H −> Y | NS3 |
| 5832 | G | T | 0.99394 | 0.00606 | 1 | 165 | 407 | 2 | GTG −> TTG | V −> L | NS3 |
| 6106 | A | G | 0.99387 | 0.00613 | 1 | 163 | 498 | 1 | TAC −> TGC | Y −> C | NS3 |
| 6187 | G | A | 0.99379 | 0.00621 | 1 | 161 | 525 | 1 | AGG −> AAG | R −> K | NS3 |
| 6223 | A | G | 0.98742 | 0.01258 | 2 | 159 | 537 | 1 | AAA −> AGA | K −> R | NS3 |
| 6277 | C | T | 0.99375 | 0.00625 | 1 | 160 | 555 | 1 | ACC −> ATC | T −> I | NS3 |
| 6306 | G | A | 0.99371 | 0.00629 | 1 | 159 | 565 | 1 | GGC −> AGC | G −> S | NS3 |
| 6310 | C | T | 0.99371 | 0.00629 | 1 | 159 | 566 | 1 | ACG −> ATG | T −> M | NS3 |
| 6327 | A | C | 0.89308 | 0.10692 | 17 | 159 | 572 | 2 | ATG −> CTG | M −> L | NS3 |
| 6364 | A | G | 0.99371 | 0.00629 | 1 | 159 | 584 | 1 | CAC −> CGC | H −> R | NS3 |
| 6453 | G | T | 0.98773 | 0.01227 | 2 | 163 | 614 | 1 | GCT −> TCT | A −> S | NS3 |
| 6468 | G | A | 0.99371 | 0.00629 | 1 | 159 | 2 | 1 | GCG −> ACG | A −> T | NS4A |
| 6471 | G | A | 0.99355 | 0.00645 | 1 | 155 | 3 | 1 | GCT −> ACT | A −> T | NS4A |
| 6474 | T | C | 0.97987 | 0.02013 | 3 | 149 | 4 | 1 | TTT −> CTT | F −> L | NS4A |
| 6481 | T | A | 0.99324 | 0.00676 | 1 | 148 | 6 | 1 | GTG −> GAG | V −> E | NS4A |
| 6485 | G | C | 0.99324 | 0.00676 | 1 | 148 | 7 | 3 | ATG −> ATC | M −> I | NS4A |
| 6487 | A | G | 0.99324 | 0.00676 | 1 | 148 | 8 | 1 | GAA −> GGA | E −> G | NS4A |
| 6592 | C | T | 0.99333 | 0.00667 | 1 | 150 | 43 | 1 | GCC −> GTC | A −> V | NS4A |
| 6598 | C | T | 0.99338 | 0.00662 | 1 | 151 | 45 | 1 | GCG −> GTG | A −> V | NS4A |
| 6606 | T | A | 0.95946 | 0.04054 | 6 | 148 | 48 | 2 | TTG −> ATG | L −> M | NS4A |
| 6866 | G | A | 0.99371 | 0.00629 | 1 | 159 | 7 | 3 | ATG −> ATA | M −> I | 2K |
| 6889 | T | C | 0.99371 | 0.00629 | 1 | 159 | 15 | 1 | GTA −> GCA | V −> A | 2K |
| 6894 | C | T | 0.99371 | 0.00629 | 1 | 159 | 17 | 1 | CTT −> TTT | L −> F | 2K |
| 6906 | A | G | 0.99371 | 0.00629 | 1 | 159 | 21 | 1 | ATT −> GTT | I −> V | 2K |
| 6934 | A | G | 0.99371 | 0.00629 | 1 | 159 | 7 | 1 | GAG −> GGG | E −> G | NS4B |
| 6957 | C | T | 0.99371 | 0.00629 | 1 | 159 | 15 | 1 | CAT −> TAT | H −> Y | NS4B |
| 6963 | A | C | 0.99371 | 0.00629 | 1 | 159 | 17 | 2 | ATG −> CTG | M −> L | NS4B |
| 6970 | G | A | 0.99371 | 0.00629 | 1 | 159 | 19 | 1 | AGG −> AAG | R −> K | NS4B |
| 6977 | G | T | 0.99371 | 0.00629 | 1 | 159 | 21 | 2 | GAG −> GAT | E −> D | NS4B |
| 6984 | G | A | 0.99371 | 0.00629 | 1 | 159 | 24 | 1 | GCA −> ACA | A −> T | NS4B |
| 6991 | T | C | 0.99383 | 0.00617 | 1 | 162 | 26 | 1 | ATA −> ACA | I −> T | NS4B |
| 6992 | A | G | 0.96296 | 0.03704 | 6 | 162 | 26 | 3 | ATA −> ATG | I −> M | NS4B |
| 6996 | T | A | 0.99383 | 0.00617 | 1 | 162 | 28 | 1 | TTC −> ATC | F −> I | NS4B |
| 7206 | A | G | 0.9932 | 0.0068 | 1 | 147 | 98 | 2 | ATA −> GTA | I −> V | NS4B |
| 7208 | A | G | 0.9932 | 0.0068 | 1 | 147 | 98 | 3 | ATA −> ATG | I −> M | NS4B |
| 7395 | A | G | 0.99329 | 0.00671 | 1 | 149 | 161 | 1 | ACA −> GCA | T −> A | NS4B |
| 7441 | T | C | 0.99296 | 0.00704 | 1 | 142 | 176 | 1 | ATA −> ACA | I −> T | NS4B |
| 7442 | A | G | 0.97902 | 0.02098 | 3 | 143 | 176 | 3 | ATA −> ATG | I −> M | NS4B |
| 7471 | C | T | 0.99296 | 0.00704 | 1 | 142 | 186 | 1 | TCG −> TTG | S −> L | NS4B |
| 7519 | C | G | 0.99301 | 0.00699 | 1 | 143 | 202 | 1 | GCA −> GGA | A −> G | NS4B |
| 7633 | C | T | 0.99342 | 0.00658 | 1 | 152 | 240 | 1 | ACA −> ATA | T −> I | NS4B |
| 7690 | T | C | 0.99333 | 0.00667 | 1 | 150 | 8 | 1 | CTG −> CCG | L −> P | NS5 |
| 7743 | T | C | 0.99281 | 0.00719 | 1 | 139 | 26 | 1 | TCC −> CCC | S −> P | NS5 |
| 7755 | T | A | 0.99286 | 0.00714 | 1 | 140 | 30 | 1 | TCA −> ACA | S −> T | NS5 |
| 7773 | T | A | 0.99291 | 0.00709 | 1 | 141 | 36 | 1 | TGC −> AGC | C −> S | NS5 |
| 7824 | C | T | 0.99281 | 0.00719 | 1 | 139 | 53 | 1 | CAT −> TAT | H −> Y | NS5 |
| 7887 | T | C | 0.96689 | 0.03311 | 5 | 151 | 74 | 1 | TAT −> CAT | Y −> H | NS5 |
| 7939 | C | T | 0.99333 | 0.00667 | 1 | 150 | 91 | 1 | GCC −> GTC | A −> V | NS5 |
| 7996 | A | G | 0.99265 | 0.00735 | 1 | 136 | 110 | 1 | CAT −> CGT | H −> R | NS5 |
| 8070 | A | G | 0.9927 | 0.0073 | 1 | 137 | 135 | 2 | ATG −> GTG | M −> V | NS5 |
| 8082 | C | T | 0.9854 | 0.0146 | 2 | 137 | 139 | 1 | CCG −> TCG | P −> S | NS5 |
| 8170 | T | C | 0.9927 | 0.0073 | 1 | 137 | 168 | 1 | GTG −> GCG | V −> A | NS5 |
| 8188 | A | G | 0.98684 | 0.01316 | 2 | 152 | 174 | 1 | AAA −> AGA | K −> R | NS5 |
| 8250 | C | A | 0.94937 | 0.01266 | 2 | 158 | 195 | 2 | CTG −> ATG | L −> M | NS5 |
| 8271 | T | C | 0.97468 | 0.02532 | 4 | 158 | 202 | 1 | TAT −> CAT | Y −> H | NS5 |
| 8403 | G | T | 0.99338 | 0.00662 | 1 | 151 | 246 | 2 | GGG −> TGG | G −> W | NS5 |
| 8406 | C | T | 0.99338 | 0.00662 | 1 | 151 | 247 | 1 | CCT −> TCT | P −> S | NS5 |
| 8439 | A | G | 0.99338 | 0.00662 | 1 | 151 | 258 | 1 | AAT −> GAT | N −> D | NS5 |
| 8467 | T | C | 0.98182 | 0.01818 | 3 | 165 | 267 | 1 | GTA −> GCA | V −> A | NS5 |
| 8473 | G | A | 0.99394 | 0.00606 | 1 | 165 | 269 | 1 | TGC −> TAC | C −> Y | NS5 |
| 8505 | A | G | 0.99394 | 0.00606 | 1 | 165 | 280 | 1 | AAC −> GAC | N −> D | NS5 |
| 8550 | T | G | 0.98788 | 0.01212 | 2 | 165 | 295 | 1 | TTT −> GTT | F −> V | NS5 |
| 8599 | A | T | 0.99351 | 0.00649 | 1 | 154 | 311 | 1 | GAG −> GTG | E −> V | NS5 |
| 8600 | G | T | 0.99351 | 0.00649 | 1 | 154 | 311 | 2 | GAG −> GAT | E −> D | NS5 |
| 8631 | A | G | 0.66234 | 0.33766 | 52 | 154 | 322 | 2 | ATA −> GTA | I −> V | NS5 |
| 8647 | G | A | 0.99359 | 0.00641 | 1 | 156 | 327 | 1 | AGG −> AAG | R −> K | NS5 |
| 8676 | A | G | 0.99359 | 0.00641 | 1 | 156 | 337 | 1 | ACT −> GCT | T −> A | NS5 |
| 8794 | G | C | 0.99394 | 0.00606 | 1 | 165 | 376 | 1 | AGC −> ACC | S −> T | NS5 |
| 8817 | A | C | 0.98795 | 0.01205 | 2 | 166 | 384 | 1 | AAA −> CAA | K −> Q | NS5 |
| 8823 | C | A | 0.99398 | 0.00602 | 1 | 166 | 386 | 2 | CTA −> ATA | L −> I | NS5 |
| 8830 | A | G | 0.98795 | 0.01205 | 2 | 166 | 388 | 1 | AAA −> AGA | K −> R | NS5 |
| 8832 | C | T | 0.99398 | 0.00602 | 1 | 166 | 389 | 1 | CAC −> TAC | H −> Y | NS5 |
| 8980 | A | G | 0.9932 | 0.0068 | 1 | 147 | 438 | 1 | AAG −> AGG | K −> R | NS5 |
| 8991 | C | T | 0.9375 | 0.0625 | 9 | 144 | 442 | 1 | CAC −> TAC | H −> Y | NS5 |
| 8992 | A | G | 0.9932 | 0.0068 | 1 | 147 | 442 | 1 | CAC −> CGC | H −> R | NS5 |
| 9117 | T | C | 0.99363 | 0.00637 | 1 | 157 | 484 | 1 | TTT −> CTT | F −> L | NS5 |
| 9240 | C | A | 0.87121 | 0.00758 | 1 | 132 | 525 | 1 | CGC −> AGC | R −> S | NS5 |
| 9240 | C | T | 0.87121 | 0.12121 | 16 | 132 | 525 | 1 | CGC −> TGC | R −> C | NS5 |
| 9244 | T | C | 0.99242 | 0.00758 | 1 | 132 | 526 | 1 | ATA −> ACA | I −> T | NS5 |
| 9302 | G | T | 0.99275 | 0.00725 | 1 | 138 | 545 | 2 | AGG −> AGT | R −> S | NS5 |
| 9327 | A | G | 0.93939 | 0.06061 | 8 | 132 | 554 | 1 | ATC −> GTC | I −> V | NS5 |
| 9346 | A | G | 0.99254 | 0.00746 | 1 | 134 | 560 | 1 | AAA −> AGA | K −> R | NS5 |
| 9406 | A | G | 0.98693 | 0.01307 | 2 | 153 | 580 | 1 | AAG −> AGG | K −> R | NS5 |
| 9591 | T | C | 0.9708 | 0.0292 | 4 | 137 | 642 | 1 | TCA −> CCA | S −> P | NS5 |
| 9595 | A | G | 0.9927 | 0.0073 | 1 | 137 | 643 | 1 | GAG −> GGG | E −> G | NS5 |
| 9604 | C | A | 0.9927 | 0.0073 | 1 | 137 | 646 | 1 | ACC −> AAC | T −> N | NS5 |
| 9616 | A | G | 0.9854 | 0.0146 | 2 | 137 | 650 | 1 | CAG −> CGG | Q −> R | NS5 |
| 9619 | G | A | 0.9927 | 0.0073 | 1 | 137 | 651 | 1 | AGC −> AAC | S −> N | NS5 |
| 9627 | T | C | 0.9854 | 0.0146 | 2 | 137 | 654 | 2 | TGG −> CGG | W −> R | NS5 |
| 9775 | A | T | 0.99194 | 0.00806 | 1 | 124 | 703 | 1 | GAC −> GTC | D −> V | NS5 |
| 9840 | T | C | 0.99194 | 0.00806 | 1 | 124 | 725 | 1 | TCC −> CCC | S −> P | NS5 |
| 10090 | C | T | 0.84667 | 0.15333 | 23 | 150 | 808 | 1 | ACT −> ATT | T −> I | NS5 |
| 10101 | C | A | 0.9932 | 0.0068 | 1 | 147 | 812 | 1 | CTT −> ATT | L −> I | NS5 |
| 10155 | A | G | 0.99301 | 0.00699 | 1 | 143 | 830 | 1 | ACC −> GCC | T −> A | NS5 |
| 10164 | A | G | 0.91667 | 0.08333 | 12 | 144 | 833 | 1 | ACG −> GCG | T −> A | NS5 |
| 10165 | C | T | 0.99306 | 0.00694 | 1 | 144 | 833 | 1 | ACG −> ATG | T −> M | NS5 |
| 10221 | C | T | 0.99301 | 0.00699 | 1 | 143 | 852 | 1 | CTC −> TTC | L −> F | NS5 |
| 10295 | A | G | 0.98611 | 0.01389 | 2 | 144 | 876 | 3 | ATA −> ATG | I −> M | NS5 |
| 10301 | T | G | 0.64336 | 0.35664 | 51 | 143 | 878 | 2 | GAT −> GAG | D −> E | NS5 |
| 10315 | T | C | 0.99301 | 0.00699 | 1 | 143 | 883 | 1 | ATG −> ACG | M −> T | NS5 |

TABLE 9
Substitution rates across the 174 genomes analyzed (110 of which were sequenced).

| | Observed rate, per available base | | | | | | Mean rate (BEAST), substitutions/site/year | | | |
|---|---|---|---|---|---|---|---|---|---|---|
| | | 1st | 2nd | 3rd | | 4-fold | 1st | 2nd | 3rd | 5′ and |
| | All | codon pos | codon pos | codon pos | 3′ UTR | deg sites | codon pos | codon pos | codon pos | 3′ UTRs |
| A > C | 3.72E−03 | 4.78E−03 | 0.00E+00 | 6.95E−03 | 0.00E+00 | 1.27E−02 | 4.23E−05 | 1.69E−05 | 1.22E−04 | 2.91E−04 |
| C > A | 9.63E−03 | 6.77E−03 | 2.55E−03 | 1.71E−02 | 0.00E+00 | 2.31E−02 | 7.50E−05 | 1.92E−05 | 1.20E−04 | 2.97E−04 |
| A > T | 9.13E−03 | 2.87E−03 | 3.31E−03 | 2.32E−02 | 8.85E−03 | 3.59E−02 | 4.65E−05 | 2.17E−05 | 2.08E−04 | 1.32E−04 |
| T > A | 1.13E−02 | 1.26E−02 | 1.03E−03 | 2.52E−02 | 0.00E+00 | 4.07E−02 | 8.69E−05 | 2.01E−05 | 2.65E−04 | 1.79E−04 |
| C > G | 4.19E−04 | 0.00E+00 | 1.28E−03 | 0.00E+00 | 0.00E+00 | 0.00E+00 | 3.78E−07 | 3.21E−05 | 2.64E−05 | 3.24E−04 |
| G > C | 3.80E−03 | 0.00E+00 | 5.27E−03 | 4.97E−03 | 0.00E+00 | 9.95E−03 | 1.81E−07 | 3.32E−05 | 2.29E−05 | 2.76E−04 |
| G > T | 5.70E−03 | 3.25E−03 | 1.32E−03 | 1.09E−02 | 7.75E−03 | 1.74E−02 | 1.98E−05 | 6.52E−06 | 1.00E−04 | 2.61E−04 |
| T > G | 4.77E−03 | 1.80E−03 | 0.00E+00 | 1.33E−02 | 0.00E+00 | 2.33E−02 | 4.37E−05 | 5.14E−06 | 1.50E−04 | 4.06E−04 |
| A > G | 5.95E−02 | 2.20E−02 | 2.65E−02 | 1.41E−01 | 5.31E−02 | 1.37E−01 | 2.01E−04 | 2.14E−04 | 1.58E−03 | 1.08E−03 |
| G > A | 4.72E−02 | 1.22E−02 | 1.45E−02 | 1.17E−01 | 2.33E−02 | 1.59E−01 | 1.70E−04 | 2.51E−04 | 1.34E−03 | 9.38E−04 |
| C > T | 1.24E−01 | 7.61E−02 | 2.42E−02 | 2.59E−01 | 4.20E−02 | 2.52E−01 | 8.99E−04 | 3.36E−04 | 3.10E−03 | 1.02E−03 |
| T > C | 1.21E−01 | 7.75E−02 | 2.77E−02 | 3.02E−01 | 8.82E−02 | 3.28E−01 | 9.48E−04 | 2.74E−04 | 4.01E−03 | 1.35E−03 |

TABLE 10
Unique viral contigs assembled from 8 mosquito pools. Includes the best hit of each
contig according to a BLASTN/BLASTX search and information scoring the hit.

| Contig name | GI/Accession | E-value | Algorithm |
|---|---|---|---|
| USA_2016_FL-08-MOS.comp510_c0_seq1 | gi\|1110865531\|gb\|APG77775.1\| | 1.31E−12 | blastx |
| USA_2016_FL-02-MOS.comp71_c0_seq1 | gi\|1110865672\|gb\|APG77877.1\| | 2.94E−109 | blastx |
| USA_2016_FL-06-MOS.comp91_c0_seq1 | gi\|1069431584\|gb\|AOR51365.1\| | 6.92E−28 | blastx |
| USA_2016_FL-06-MOS.comp100_c0_seq2 | gi\|1069431584\|gb\|AOR51365.1\| | 3.16E−09 | blastx |
| USA_2016_FL-01-MOS.comp564_c0_seq1 | gi\|1069431612\|gb\|AOR51381.1\| | 3.37E−11 | blastx |
| USA_2016_FL-04-MOS.comp698_c0_seq1 | gi\|1132371544\|ref\|YP_009337412.1\| | 1.09E−11 | blastx |
| USA_2016_FL-08-MOS.comp384_c0_seq1 | gi\|1132371544\|ref\|YP_009337412.1\| | 4.34E−10 | blastx |
| USA_2016_FL-02-MOS.comp907_c0_seq1 | gi\|1069431584\|gb\|AOR51365.1\| | 2.46E−19 | blastx |
| USA_2016_FL-02-MOS.comp29_c0_seq4 | gi\|1069431609\|gb\|AOR51378.1\| | 7.55E−64 | blastx |
| USA_2016_FL-08-MOS.comp2327_c0_seq1 | gi\|1069431612\|gb\|AOR51381.1\| | 3.51E−09 | blastx |
| USA_2016_FL-08-MOS.comp654_c0_seq1 | gi\|1110865383\|gb\|APG77663.1\| | 1.59E−12 | blastx |
| USA_2016_FL-08-MOS.comp182_c0_seq1 | gi\|1069431590\|gb\|AOR51366.1\| | 5.47E−95 | blastx |
| USA_2016_FL-06-MOS.comp191_c0_seq1 | gi\|1069431583\|gb\|AOR51364.1\| | 4.46E−48 | blastx |
| USA_2016_FL-02-MOS.comp34_c0_seq1 | gi\|1110865676\|gb\|APG77879.1\| | 7.63E−121 | blastx |
| USA_2016_FL-06-MOS.comp118_c0_seq1 | gi\|1069431583\|gb\|AOR51364.1\| | 1.47E−52 | blastx |
| USA_2016_FL-08-MOS.comp1176_c0_seq1 | gi\|1069431583\|gb\|AOR51354.1\| | 2.26E−13 | blastx |
| USA_2016_FL-06-MOS.comp185_c0_seq1 | gi\|1003096207\|gb\|AMO03220.1\| | 1.54E−18 | blastx |
| USA_2016_FL-02-MOS.comp157_c0_seq1 | gi\|1110865714\|gb\|APG77898.1\| | 0 | blastx |
| USA_2016_FL-08-MOS.comp822_c0_seq1 | gi\|1110865383\|gb\|APG77663.1\| | 2.27E−26 | blastx |
| USA_2016_FL-08-MOS.comp1485_c0_seq1 | gi\|1003096207\|gb\|AMO03220.1\| | 2.72E−29 | blastx |
| USA_2016_FL-08-MOS.comp621_c0_seq1 | gi\|1012303438\|gb\|AMS24261.1\| | 3.14E−25 | blastx |
| USA_2016_FL-06-MOS.comp254_c0_seq1 | gi\|1110865383\|gb\|APG77563.1\| | 1.11E−22 | blastx |
| USA_2016_FL-04-MOS.comp15_c0_seq1 | gi\|1126394488\|ref\|YP_009333370.1\| | 2.05E−163 | blastx |
| USA_2016_FL-08-MOS.comp948_c0_seq1 | gi\|1132372055\|ref\|YP_009337823.1\| | 6.28E−40 | blastx |
| USA_2016_FL-08-MOS.comp605_c0_seq1 | gi\|1003096207\|gb\|AMO03220.1\| | 1.20E−81 | blastx |
| USA_2016_FL-06-MOS.comp176_c0_seq1 | gi\|1003096207\|gb\|AMO03220.1\| | 4.48E−48 | blastx |
| USA_2016_FL-06-MOS.comp131_c0_seq1 | gi\|1069431583\|gb\|AOR51364.1\| | 1.40E−28 | blastx |
| USA_2016_FL-06-MOS.comp279_c0_seq1 | gi\|1003096207\|gb\|AMO03220.1\| | 5.61E−26 | blastx |
| USA_2016_FL-08-MOS.comp326_c0_seq1 | gi\|1069431583\|gb\|AOR51364.1\| | 1.61E−64 | blastx |
| USA_2016_FL-02-MOS.comp79_c0_seq1 | gi\|1110866469\|gb\|APG78322.1\| | 6.30E−174 | blastx |
| USA_2016_FL-06-MOS.comp733_c0_seq2 | gi\|870898376\|gb\|AKP18601.1\| | 9.85E−09 | blastx |
| USA_2016_FL-08-MOS.comp1073_c0_seq1 | gi\|1012303438\|gb\|AMS24261.1\| | 6.31E−34 | blastx |
| USA_2016_FL-02-MOS.comp52_c0_seq1 | gi\|752455575\|gb\|AJG39093.1\| | 2.22E−169 | blastx |
| USA_2016_FL-08-MOS.comp6_c0_seq1 | gi\|1069431616\|gb\|AOR51384.1\| | 1.78E−110 | blastx |
| USA_2016_FL-02-MOS.comp52_c0_seq2 | gi\|752455575\|gb\|AJG39093.1\| | 5.29E−128 | blastx |
| USA_2016_FL-04-MOS.comp458_c0_seq1 | gi\|752455575\|gb\|AJG39093.1\| | 2.08E−152 | blastx |
| USA_2016_FL-06-MOS.comp96_c0_seq1 | gi\|1069431583\|gb\|AOR51364.1\| | 5.08E−50 | blastx |
| USA_2016_FL-04-MOS.comp600_c0_seq1 | gi\|1110864621\|gb\|APG77144.1\| | 5.00E−43 | blastx |
| USA_2016_FL-08-MOS.comp1421_c0_seq1 | gi\|1012303438\|gb\|AMS24261.1\| | 5.13E−45 | blastx |
| USA_2016_FL-08-MOS.comp2124_c0_seq1 | gi\|1012303438\|gb\|AMS24261.1\| | 6.16E−60 | blastx |
| USA_2016_FL-03-MOS.comp213_c0_seq1 | gi\|752455575\|gb\|AJG39093.1\| | 6.71E−41 | blastx |
| USA_2016_FL-04-MOS.comp473_c0_seq1 | gi\|545716055\|gb\|AGW51782.1\| | 1.74E−99 | blastx |
| USA_2016_FL-05-MOS.comp257_c0_seq1 | gi\|666396950\|gb\|KJ476731.1\| | 1.09E−164 | blastn |
| USA_2016_FL-03-MOS.comp134_c0_seq1 | gi\|766989345\|gb\|KP642128.1\| | 4.13E−128 | blastn |
| USA_2016_FL-01-MOS.comp321_c0_seq1 | gi\|1110866630\|gb\|KX884274.1\| | 0 | blastn |
| USA_2016_FL-06-MOS.comp924_c0_seq1 | gi\|544185881\|dbj\|AB813769.1\| | 4.09E−118 | blastn |
| USA_2016_FL-03-MOS.comp76_c0_seq1 | gi\|766989345\|gb\|KP642128.1\| | 0 | blastn |
| USA_2016_FL-05-MOS.comp228_c0_seq1 | gi\|678193589\|gb\|KJ741267.1\| | 0 | blastn |
| USA_2016_FL-05-MOS.comp71_c0_seq1 | gi\|666396950\|gb\|KJ476731.1\| | 0 | blastn |
| USA_2016_FL-01-MOS.comp178_c0_seq1 | gi\|678193589\|gb\|KJ741267.1\| | 0 | blastn |
| USA_2016_FL-05-MOS.comp168_c0_seq1 | gi\|666396950\|gb\|KJ476731.1\| | 0 | blastn |
| USA_2016_FL-03-MOS.comp332_c0_seq1 | gi\|766989345\|gb\|KP642128.1\| | 1.19E−148 | blastn |
| USA_2016_FL-05-MOS.comp85_c0_seq1 | gi\|544185965\|dbj\|AB813811.1\| | 0 | blastn |
| USA_2016_FL-05-MOS.comp194_c0_seq1 | gi\|256599193\|gb\|GQ165810.1\| | 0 | blastn |
| USA_2016_FL-01-MOS.comp508_c0_seq1 | gi\|1110866630\|gb\|KX884274.1\| | 9.39E−160 | blastn |
| USA_2016_FL-01-MOS.comp213_c0_seq1 | gi\|678193589\|gb\|KJ741267.1\| | 0 | blastn |
| USA_2016_FL-01-MOS.comp641_c0_seq1 | gi\|1057718472\|gb\|KU936054.1\| | 4.12E−148 | blastn |
| USA_2016_FL-01-MOS.comp52_c0_seq1 | gi\|870898375\|gb\|KR003785.1\| | 0 | blastn |
| USA_2016_FL-01-MOS.comp538_c0_seq1 | gi\|1110866708\|gb\|KX884304.1\| | 0 | blastn |
| USA_2016_FL-01-MOS.comp38_c0_seq1 | gi\|870898372\|gb\|KR003784.1\| | 0 | blastn |
| USA_2016_FL-08-MOS.comp333_c0_seq1 | gi\|1057718472\|gb\|KU936054.1\| | 0 | blastn |
| USA_2016_FL-01-MOS.comp329_c0_seq1 | gi\|666396950\|gb\|KJ476731.1\| | 0 | blastn |
| USA_2016_FL-05-MOS.comp345_c0_seq1 | gi\|1057718472\|gb\|KU936054.1\| | 0 | blastn |
| USA_2016_FL-05-MOS.comp140_c0_seq1 | gi\|666396950\|gb\|KJ476731.1\| | 0 | blastn |
| USA_2016_FL-05-MOS.comp110_c0_seq1 | gi\|1041156621\|ref\|YP_009259257.1\| | 0 | blastx |
| USA_2016_FL-05-MOS.comp45_c0_seq1 | gi\|1041156634\|ref\|YP_009259316.1\| | 0 | blastx |
| USA_2016_FL-08-MOS.comp1213_c0_seq1 | gi\|1057718472\|gb\|KU936054.1\| | 1.24E−173 | blastn |
| USA_2016_FL-08-MOS.comp710_c0_seq1 | gi\|1057718472\|gb\|KU936054.1\| | 1.63E−177 | blastn |
| USA_2016_FL-08-MOS.comp1056_c0_seq1 | gi\|336190\|gb\|M91671.1\|YFVCFAPP | 5.13E−152 | blastn |
| USA_2016_FL-08-MOS.comp479_c0_seq1 | gi\|336190\|gb\|M91671.1\|YFVCFAPP | 0 | blastn |
| USA_2016_FL-01-MOS.comp245_c0_seq1 | gi\|1057718472\|gb\|KU936054.1\| | 0 | blastn |
| USA_2016_FL-05-MOS.comp618_c0_seq1 | gi\|1057718478\|gb\|KU935057.1\| | 6.82E−161 | blastn |
| USA_2016_FL-05-MOS.comp97_c0_seq1 | gi\|256599194\|gb\|ACV04606.1\| | 0 | blastx |
| USA_2016_FL-01-MOS.comp822_c0_seq1 | gi\|1110866630\|gb\|KX884274.1\| | 2.32E−150 | blastn |
| USA_2016_FL-01-MOS.comp35_c0_seq1 | gi\|1057718478\|gb\|KU936057.1\| | 0 | blastn |
| USA_2016_FL-08-MOS.comp1607_c0_seq1 | gi\|336190\|gb\|M91671.1\|YFVCFAPP | 1.09E−153 | blastn |
| USA_2016_FL-01-MOS.comp26_c0_seq1 | gi\|870898559\|gb\|KR003802.1\| | 0 | blastn |
| USA_2016_FL-05-MOS.comp53_c0_seq1 | gi\|251823474\|dbj\|BAH83684.1\| | 0 | blastx |
| USA_2016_FL-06-MOS.comp252_c0_seq1 | gi\|1057718472\|gb\|KU936054.1\| | 1.89E−166 | blastn |
| USA_2016_FL-01-MOS.comp22_c0_seq1 | gi\|870898556\|gb\|KR003801.1\| | 0 | blastn |
| USA_2016_FL-06-MOS.comp841_c0_seq1 | gi\|1120603288\|gb\|KY325478.1\| | 6.61E−141 | blastn |
| USA_2016_FL-06-MOS.comp1096_c0_seq1 | gi\|1145144303\|gb\|KY606273.1\| | 2.98E−159 | blastn |
| USA_2016_FL-06-MOS.comp557_c0_seq1 | gi\|1120603286\|gb\|KY325477.1\| | 0 | blastn |
| USA_2016_FL-06-MOS.comp614_c0_seq1 | gi\|1145144305\|gb\|KY606274.1\| | 1.99E−111 | blastn |

| Contig name | Hit description |
|---|---|
| USA_2016_FL-08-MOS.comp510_c0_seq1 | RdRp [Wuhan insect virus 9] |
| USA_2016_FL-02-MOS.comp71_c0_seq1 | polymerase PB2 [Jingshan Fly Virus 1] |
| USA_2016_FL-06-MOS.comp91_c0_seq1 | putative capsid [Anopheles totivirus] |
| USA_2016_FL-06-MOS.comp100_c0_seq2 | putative capsid [Anopheles totivirus] |
| USA_2016_FL-01-MOS.comp564_c0_seq1 | putative nucleoprotein [Gambie virus] |
| USA_2016_FL-04-MOS.comp698_c0_seq1 | RdRp [Hubei virga-like virus 2] |
| USA_2016_FL-08-MOS.comp384_c0_seq1 | RdRp [Hubei virga-like virus 2] |
| USA_2016_FL-02-MOS.comp907_c0_seq1 | putative capsid [Anopheles totivirus] |
| USA_2016_FL-02-MOS.comp29_c0_seq4 | RdRp [Gambie virus] |
| USA_2016_FL-08-MOS.comp2327_c0_seq1 | putative nucleoprotein [Gambie virus] |
| USA_2016_FL-08-MOS.comp654_c0_seq1 | hypothetical protein [Hubei virga-like virus 2] |
| USA_2016_FL-08-MOS.comp182_c0_seq1 | RdRp [Bolahun virus variant 1] |
| USA_2016_FL-06-MOS.comp191_c0_seq1 | RdRp [Anopheles totivirus] |
| USA_2016_FL-02-MOS.comp34_c0_seq1 | nucleocapsid protein [Jingshan Fly Virus 1] |
| USA_2016_FL-06-MOS.comp118_c0_seq1 | RdRp [Anopheles totivirus] |
| USA_2016_FL-08-MOS.comp1176_c0_seq1 | RdRp [Anopheles totivirus] |
| USA_2016_FL-06-MOS.comp185_c0_seq1 | putative polyprotein, partial [Blackford virus] |
| USA_2016_FL-02-MOS.comp157_c0_seq1 | polymerase PB2 [Jingshan Fly Virus 1] |
| USA_2016_FL-08-MOS.comp822_c0_seq1 | hypothetical protein [Hubei virga-like virus 2] |
| USA_2016_FL-08-MOS.comp1485_c0_seq1 | putative polyprotein, partial [Blackford virus] |
| USA_2016_FL-08-MOS.comp621_c0_seq1 | polyprotein [Xishuangbanna aedes flavivirus] |
| USA_2016_FL-06-MOS.comp254_c0_seq1 | hypothetical protein [Hubei virga-like virus 2] |
| USA_2016_FL-04-MOS.comp15_c0_seq1 | RdRp [Beihai barnacle virus 12] |
| USA_2016_FL-08-MOS.comp948_c0_seq1 | hypothetical protein [Hubei virga-like virus 12] |
| USA_2016_FL-08-MOS.comp605_c0_seq1 | putative polyprotein, partial [Blackford virus] |
| USA_2016_FL-06-MOS.comp176_c0_seq1 | putative polyprotein, partial [Blackford virus] |
| USA_2016_FL-06-MOS.comp131_c0_seq1 | RdRp [Anopheles totivirus] |
| USA_2016_FL-06-MOS.comp279_c0_seq1 | putative polyprotein, partial [Blackford virus] |
| USA_2016_FL-08-MOS.comp326_c0_seq1 | RdRp [Anopheles totivirus] |
| USA_2016_FL-02-MOS.comp79_c0_seq1 | RdRp [Hubei partiti-like virus 34] |
| USA_2016_FL-06-MOS.comp733_c0_seq2 | Nucleocapsid [Phasi Charoen-like virus] |
| USA_2016_FL-08-MOS.comp1073_c0_seq1 | polyprotein [Xishuangbanna aedes flavivirus] |
| USA_2016_FL-02-MOS.comp52_c0_seq1 | PB1 [Wuhan Mosquito Virus 5] |
| USA_2016_FL-08-MOS.comp6_c0_seq1 | ORF1 [Chaq virus-like 1] |
| USA_2016_FL-02-MOS.comp52_c0_seq2 | PB1 [Wuhan Mosquito Virus 5] |
| USA_2016_FL-04-MOS.comp458_c0_seq1 | PB1 [Wuhan Mosquito Virus 5] |
| USA_2016_FL-06-MOS.comp96_c0_seq1 | RdRp [Anopheles totivirus] |
| USA_2016_FL-04-MOS.comp600_c0_seq1 | RNA-dependent RNA polymerase, partial [Zhejiang mosqu [symbol] |
| USA_2016_FL-08-MOS.comp1421_c0_seq1 | polyprotein [Xishuangbanna aedes flavivirus] |
| USA_2016_FL-08-MOS.comp2124_c0_seq1 | polyprotein [Xishuangbanna aedes flavivirus] |
| USA_2016_FL-03-MOS.comp213_c0_seq1 | PB1 [Wuhan Mosquito Virus 5] |
| USA_2016_FL-04-MOS.comp473_c0_seq1 | putative RNA-dependent RNA polymerase-like protein, pa [symbol] |
| USA_2016_FL-05-MOS.comp257_c0_seq1 | Cell fusing agent virus isolate Mex AR269 polyprotein gen [symbol] |
| USA_2016_FL-03-MOS.comp134_c0_seq1 | DsRNA virus environmental sample clone thai.aeae_conti [symbol] |
| USA_2016_FL-01-MOS.comp321_c0_seq1 | Wuhan insect virus 33 strain WHCCII11871 hypothetical p [symbol] |
| USA_2016_FL-06-MOS.comp924_c0_seq1 | Cell fusing agent virus NS3 gene for non-structural protein |
| USA_2016_FL-03-MOS.comp76_c0_seq1 | DsRNA virus environmental sample clone thai.aeae_conti [symbol] |
| USA_2016_FL-05-MOS.comp228_c0_seq1 | Cell fusing agent virus strain Galveston, complete genome |
| USA_2016_FL-05-MOS.comp71_c0_seq1 | Cell fusing agent virus isolate Mex AR269 polyprotein gen [symbol] |
| USA_2016_FL-01-MOS.comp178_c0_seq1 | Cell fusing agent virus strain Galveston, complete genome |
| USA_2016_FL-05-MOS.comp168_c0_seq1 | Cell fusing agent virus isolate Mex AR269 polyprotein gen [symbol] |
| USA_2016_FL-03-MOS.comp332_c0_seq1 | DsRNA virus environmental sample clone thai.aeae_conti [symbol] |
| USA_2016_FL-05-MOS.comp85_c0_seq1 | Cell fusing agent virus NS5 gene for non-structural protein |
| USA_2016_FL-05-MOS.comp194_c0_seq1 | Cell fusing agent virus strain Rio Piedras02 polyprotein ge [symbol] |
| USA_2016_FL-01-MOS.comp508_c0_seq1 | Wuhan insect virus 33 strain WHCCII11871 hypothetical p [symbol] |
| USA_2016_FL-01-MOS.comp213_c0_seq1 | Cell fusing agent virus strain Galveston, complete genome |
| USA_2016_FL-01-MOS.comp641_c0_seq1 | Cell fusing agent virus strain Aag2 Bristol polyprotein gen [symbol] |
| USA_2016_FL-01-MOS.comp52_c0_seq1 | Phasi Charoen-like virus isolate Rio segment S Nucleocaps [symbol] |
| USA_2016_FL-01-MOS.comp538_c0_seq1 | Wuhan insect virus 33 strain WHYY3291 hypothetical prot [symbol] |
| USA_2016_FL-01-MOS.comp38_c0_seq1 | Phasi Charoen-like virus isolate Rio segment M glycoprote [symbol] |
| USA_2016_FL-08-MOS.comp333_c0_seq1 | Cell fusing agent virus strain Aag2 Bristol polyprotein gen [symbol] |
| USA_2016_FL-01-MOS.comp329_c0_seq1 | Cell fusing agent virus isolate Mex AR269 polyprotein gen [symbol] |
| USA_2016_FL-05-MOS.comp345_c0_seq1 | Cell fusing agent virus strain Aag2 Bristol polyprotein gen [symbol] |
| USA_2016_FL-05-MOS.comp140_c0_seq1 | Cell fusing agent virus isolate Mex AR269 polyprotein gen [symbol] |
| USA_2016_FL-05-MOS.comp110_c0_seq1 | flavivirus polyprotein [Cell fusing agent virus] |
| USA_2016_FL-05-MOS.comp45_c0_seq1 | truncated polyprotein [Cell fusing agent virus] |
| USA_2016_FL-08-MOS.comp1213_c0_seq1 | Cell fusing agent virus strain Aag2 Bristol polyprotein gen [symbol] |
| USA_2016_FL-08-MOS.comp710_c0_seq1 | Cell fusing agent virus strain Aag2 Bristol polyprotein gen [symbol] |
| USA_2016_FL-08-MOS.comp1056_c0_seq1 | Flavivirus cell fusing agent polyprotein gene, complete cd [symbol] |
| USA_2016_FL-08-MOS.comp479_c0_seq1 | Flavivirus cell fusing agent polyprotein gene, complete cd [symbol] |
| USA_2016_FL-01-MOS.comp245_c0_seq1 | Cell fusing agent virus strain Aag2 Bristol polyprotein gen [symbol] |
| USA_2016_FL-05-MOS.comp618_c0_seq1 | Phasi Charoen-like virus strain Aag2-Bristol RNA-depende [symbol] |
| USA_2016_FL-05-MOS.comp97_c0_seq1 | polyprotein, partial [Cell fusing agent virus] |
| USA_2016_FL-01-MOS.comp822_c0_seq1 | Wuhan insect virus 33 strain WHCCII11871 hypothetical p [symbol] |
| USA_2016_FL-01-MOS.comp35_c0_seq1 | Phasi Charoen-like virus strain Aag2-Bristol RNA-depende [symbol] |
| USA_2016_FL-08-MOS.comp1607_c0_seq1 | Flavivirus cell fusing agent polyprotein gene, complete cd [symbol] |
| USA_2016_FL-01-MOS.comp26_c0_seq1 | Humaita-Tubiacanga virus isolate Rio segment capsid Cap [symbol] |
| USA_2016_FL-05-MOS.comp53_c0_seq1 | polyprotein, partial [Cell fusing agent virus] |
| USA_2016_FL-06-MOS.comp252_c0_seq1 | Cell fusing agent virus strain Aag2 Bristol polyprotein gen [symbol] |
| USA_2016_FL-01-MOS.comp22_c0_seq1 | Humaita-Tubiacanga virus isolate Rio segment RNA-depe [symbol] |
| USA_2016_FL-06-MOS.comp841_c0_seq1 | Zika virus isolate Zika virus/H. sapiens-wt/USA/2016/FLWB [symbol] |
| USA_2016_FL-06-MOS.comp1096_c0_seq1 | Zika virus isolate mex48/Mexico/2016 polyprotein gene, [symbol] |
| USA_2016_FL-06-MOS.comp557_c0_seq1 | Zika virus isolate Zika virus/H. sapiens-wt/USA/2016/FLUR [symbol] |
| USA_2016_FL-06-MOS.comp614_c0_seq1 | Zika virus isolate mex39/Mexico/2016 polyprotein gene, c [symbol] |

| | Taxonomy | | Percent | Bit | |
|---|---|---|---|---|---|
| Contig name | ID | Taxonomy name | Identity | score | Length |
| USA_2016_FL-08-MOS.comp510_c0_seq1 | 1923740 | Wuhan insect virus 9 | 28.931 | 74.7 | 159 |
| USA_2016_FL-02-MOS.comp71_c0_seq1 | 1608053 | Jingshan Fly Virus 1 | 29.335 | 361 | 767 |
| USA_2016_FL-06-MOS.comp91_c0_seq1 | 1903415 | Anopheles totivirus | 29.607 | 124 | 331 |
| USA_2016_FL-06-MOS.comp100_c0_seq2 | 1903415 | Anopheles totivirus | 29.63 | 65.5 | 162 |
| USA_2016_FL-01-MOS.comp564_c0_seq1 | 1903427 | Gambie virus | 30.769 | 68.9 | 117 |
| USA_2016_FL-04-MOS.comp698_c0_seq1 | 1923335 | Hubei virga-like virus 2 | 31.429 | 71.6 | 140 |
| USA_2016_FL-08-MOS.comp384_c0_seq1 | 1923335 | Hubei virga-like virus 2 | 32.192 | 50.1 | 146 |
| USA_2016_FL-02-MOS.comp907_c0_seq1 | 1903415 | Anopheles totivirus | 32.597 | 94.4 | 181 |
| USA_2016_FL-02-MOS.comp29_c0_seq4 | 1903427 | Gambie virus | 32.818 | 243 | 582 |
| USA_2016_FL-08-MOS.comp2327_c0_seq1 | 1903427 | Gambie virus | 33.01 | 62.4 | 103 |
| USA_2016_FL-08-MOS.comp654_c0_seq1 | 1923335 | Hubei virga-like virus 2 | 36.719 | 72.8 | 128 |
| USA_2016_FL-08-MOS.comp182_c0_seq1 | 1903426 | Bolahun virus variant 1 | 37.37 | 328 | 578 |
| USA_2016_FL-06-MOS.comp191_c0_seq1 | 1903415 | Anopheles totivirus | 37.597 | 180 | 258 |
| USA_2016_FL-02-MOS.comp34_c0_seq1 | 1608053 | Jingshan Fly Virus 1 | 39.326 | 377 | 534 |
| USA_2016_FL-06-MOS.comp118_c0_seq1 | 1903415 | Anopheles totivirus | 40.316 | 195 | 253 |
| USA_2016_FL-08-MOS.comp1176_c0_seq1 | 1903415 | Anopheles totivirus | 40.541 | 76.3 | 111 |
| USA_2016_FL-06-MOS.comp185_c0_seq1 | 1807801 | Blackford virus | 41.129 | 94.7 | 124 |
| USA_2016_FL-02-MOS.comp157_c0_seq1 | 1608053 | Jingshan Fly Virus 1 | 41.341 | 547 | 716 |
| USA_2016_FL-08-MOS.comp822_c0_seq1 | 1923335 | Hubei virga-like virus 2 | 42.604 | 114 | 169 |
| USA_2016_FL-08-MOS.comp1485_c0_seq1 | 1807801 | Blackford virus | 45.038 | 121 | 131 |
| USA_2016_FL-08-MOS.comp621_c0_seq1 | 1821227 | Xishuangbanna aedes | 45.528 | 110 | 123 |
| | | flavivirus | | | |
| USA_2016_FL-06-MOS.comp254_c0_seq1 | 1923335 | Hubei virga-like virus 2 | 45.872 | 102 | 109 |
| USA_2016_FL-04-MOS.comp15_c0_seq1 | 1922356 | Beihai barnacle virus 12 | 47.048 | 485 | 525 |
| USA_2016_FL-08-MOS.comp948_c0_seq1 | 1923327 | Hubei virga-like virus 12 | 47.586 | 142 | 145 |
| USA_2016_FL-08-MOS.comp605_c0_seq1 | 1807801 | Blackford virus | 49.097 | 275 | 277 |
| USA_2016_FL-06-MOS.comp176_c0_seq1 | 1807801 | Blackford virus | 49.405 | 176 | 168 |
| USA_2016_FL-06-MOS.comp131_c0_seq1 | 1903415 | Anopheles totivirus | 50 | 118 | 118 |
| USA_2016_FL-06-MOS.comp279_c0_seq1 | 1807801 | Blackford virus | 50.877 | 115 | 114 |
| USA_2016_FL-08-MOS.comp326_c0_seq1 | 1903415 | Anopheles totivirus | 51.923 | 224 | 208 |
| USA_2016_FL-02-MOS.comp79_c0_seq1 | 1923041 | Hubei partiti-like virus 34 | 52.766 | 506 | 470 |
| USA_2016_FL-06-MOS.comp733_c0_seq2 | 1521189 | Phasi Charoen-like virus | 53.704 | 60.8 | 54 |
| USA_2016_FL-08-MOS.comp1073_c0_seq1 | 1821227 | Xishuangbanna aedes | 56.863 | 132 | 102 |
| | | flavivirus | | | |
| USA_2016_FL-02-MOS.comp52_c0_seq1 | 1608130 | Wuhan Mosquito Virus 5 | 58.711 | 504 | 419 |
| USA_2016_FL-08-MOS.comp6_c0_seq1 | 1903431 | Chaq virus-like 1 | 59.122 | 338 | 296 |
| USA_2016_FL-02-MOS.comp52_c0_seq2 | 1608130 | Wuhan Mosquito Virus 5 | 60.734 | 394 | 354 |
| USA_2016_FL-04-MOS.comp458_c0_seq1 | 1608130 | Wuhan Mosquito Virus 5 | 60.784 | 459 | 408 |
| USA_2016_FL-06-MOS.comp96_c0_seq1 | 1903415 | Anopheles totivirus | 60.839 | 181 | 143 |
| USA_2016_FL-04-MOS.comp600_c0_seq1 | 1923779 | Zhejiang mosquito virus 3 | 65.942 | 160 | 138 |
| USA_2016_FL-08-MOS.comp1421_c0_seq1 | 1821227 | Xishuangbanna aedes | 66.981 | 164 | 106 |
| | | flavivirus | | | |
| USA_2016_FL-08-MOS.comp2124_c0_seq1 | 1821227 | Xishuangbanna aedes | 68.966 | 209 | 145 |
| | | flavivirus | | | |
| USA_2016_FL-03-MOS.comp213_c0_seq1 | 1608130 | Wuhan Mosquito Virus 5 | 73.529 | 153 | 102 |
| USA_2016_FL-04-MOS.comp473_c0_seq1 | 340016 | uncultured virus | 84.865 | 315 | 185 |
| USA_2016_FL-05-MOS.comp257_c0_seq1 | 31658 | Cell fusing agent virus | 93.25 | 590 | 400 |
| USA_2016_FL-03-MOS.comp134_c0_seq1 | 1075826 | uncultured dsRNA virus | 93.354 | 468 | 316 |
| USA_2016_FL-01-MOS.comp321_c0_seq1 | 1923736 | Wuhan insect virus 33 | 93.95 | 900 | 595 |
| USA_2016_FL-06-MOS.comp924_c0_seq1 | 31658 | Cell fusing agent virus | 94.946 | 435 | 277 |
| USA_2016_FL-03-MOS.comp76_c0_seq1 | 1075826 | uncultured dsRNA virus | 95.122 | 776 | 492 |
| USA_2016_FL-05-MOS.comp228_c0_seq1 | 31658 | Cell fusing agent virus | 95.147 | 1399 | 886 |
| USA_2016_FL-05-MOS.comp71_c0_seq1 | 31658 | Cell fusing agent virus | 95.249 | 701 | 442 |
| USA_2016_FL-01-MOS.comp178_c0_seq1 | 31658 | Cell fusing agent virus | 95.281 | 706 | 445 |
| USA_2016_FL-05-MOS.comp168_c0_seq1 | 31658 | Cell fusing agent virus | 95.314 | 1288 | 811 |
| USA_2016_FL-03-MOS.comp332_c0_seq1 | 1075826 | uncultured dsRNA virus | 95.522 | 536 | 335 |
| USA_2016_FL-05-MOS.comp85_c0_seq1 | 31658 | Cell fusing agent virus | 95.781 | 765 | 474 |
| USA_2016_FL-05-MOS.comp194_c0_seq1 | 31658 | Cell fusing agent virus | 95.804 | 1155 | 715 |
| USA_2016_FL-01-MOS.comp508_c0_seq1 | 1923736 | Wuhan insect virus 33 | 96.532 | 573 | 346 |
| USA_2016_FL-01-MOS.comp213_c0_seq1 | 31658 | Cell fusing agent virus | 96.56 | 723 | 436 |
| USA_2016_FL-01-MOS.comp641_c0_seq1 | 31658 | Cell fusing agent virus | 96.584 | 534 | 322 |
| USA_2016_FL-01-MOS.comp52_c0_seq1 | 1521189 | Phasi Charoen-like virus | 96.632 | 2213 | 1336 |
| USA_2016_FL-01-MOS.comp538_c0_seq1 | 1923736 | Wuhan insect virus 33 | 96.896 | 755 | 451 |
| USA_2016_FL-01-MOS.comp38_c0_seq1 | 1521189 | Phasi Charoen-like virus | 96.952 | 6438 | 1838 |
| USA_2016_FL-08-MOS.comp333_c0_seq1 | 31658 | Cell fusing agent virus | 97.176 | 1018 | 602 |
| USA_2016_FL-01-MOS.comp329_c0_seq1 | 31658 | Cell fusing agent virus | 97.382 | 1301 | 764 |
| USA_2016_FL-05-MOS.comp345_c0_seq1 | 31658 | Cell fusing agent virus | 97.445 | 933 | 548 |
| USA_2016_FL-05-MOS.comp140_c0_seq1 | 31658 | Cell fusing agent virus | 97.529 | 900 | 526 |
| USA_2016_FL-05-MOS.comp110_c0_seq1 | 31658 | Cell fusing agent virus | 97.697 | 928 | 521 |
| USA_2016_FL-05-MOS.comp45_c0_seq1 | 31658 | Cell fusing agent virus | 97.727 | 543 | 264 |
| USA_2016_FL-08-MOS.comp1213_c0_seq1 | 31658 | Cell fusing agent virus | 97.772 | 619 | 359 |
| USA_2016_FL-08-MOS.comp710_c0_seq1 | 31658 | Cell fusing agent virus | 97.814 | 632 | 366 |
| USA_2016_FL-08-MOS.comp1056_c0_seq1 | 11093 | Flavivirus sp. | 98.089 | 547 | 314 |
| USA_2016_FL-08-MOS.comp479_c0_seq1 | 11093 | Flavivirus sp. | 98.134 | 1402 | 804 |
| USA_2016_FL-01-MOS.comp245_c0_seq1 | 31658 | Cell fusing agent virus | 98.391 | 656 | 373 |
| USA_2016_FL-05-MOS.comp618_c0_seq1 | 1521189 | Phasi Charoen-like virus | 98.471 | 577 | 327 |
| USA_2016_FL-05-MOS.comp97_c0_seq1 | 31658 | Cell fusing agent virus | 98.594 | 986 | 498 |
| USA_2016_FL-01-MOS.comp822_c0_seq1 | 1923736 | Wuhan insect virus 33 | 98.689 | 542 | 305 |
| USA_2016_FL-01-MOS.comp35_c0_seq1 | 1521189 | Phasi Charoen-like virus | 98.699 | 12006 | 6765 |
| USA_2016_FL-08-MOS.comp1607_c0_seq1 | 11093 | Flavivirus sp. | 98.714 | 553 | 311 |
| USA_2016_FL-01-MOS.comp26_c0_seq1 | 1670677 | Humaita-Tubiacanga virus | 99.119 | 2857 | 1589 |
| USA_2016_FL-05-MOS.comp53_c0_seq1 | 31658 | Cell fusing agent virus | 99.19 | 518 | 247 |
| USA_2016_FL-06-MOS.comp252_c0_seq1 | 31658 | Cell fusing agent virus | 99.392 | 595 | 329 |
| USA_2016_FL-01-MOS.comp22_c0_seq1 | 1670677 | Humaita-Tubiacanga virus | 99.424 | 5036 | 2776 |
| USA_2016_FL-06-MOS.comp841_c0_seq1 | 64320 | Zika virus | 99.642 | 510 | 279 |
| USA_2016_FL-06-MOS.comp1096_c0_seq1 | 64320 | Zika virus | 100 | 571 | 309 |
| USA_2016_FL-06-MOS.comp557_c0_seq1 | 64320 | Zika virus | 100 | 841 | 455 |
| USA_2016_FL-06-MOS.comp614_c0_seq1 | 64320 | Zika virus | 100 | 412 | 223 |

De-duplicated (unique) viral contigs from 8 mosquito pools and their best hit from BLASTN or BLASTX. Viral contigs may have been found in other mosquito pools but are considered duplicates of ones shown here. Viral contigs with <80% identity, as determined by BLASTX, are considered likely novel contigs. [symbol]
indicates data missing or illegible when filed

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

### REFERENCES

Akiyama, Benjamin M., Hannah M. Laurence, Aaron R. Massey, David A. Costantino, Xuping Xie, Yujiao Yang, Pei-Yong Shi, Jay C. Nix, J. David Beckham, and Jeffrey S. Kieft. 2016. “Zika Virus Produces Noncoding RNAs Using a Multi-Pseudoknot Structure That Confounds a Cellular Exonuclease.” Science 354 (6316): 1148-52.
Altschul, S. F., T. L. Madden, A. A. Schaffer, J. Zhang, Z. Zhang, W. Miller, and D. J. Lipman. 1997. “Gapped BLAST and PSI-BLAST: A New Generation of Protein Database Search Programs.” Nucleic Acids Research 25 (17): 3389-3402.
Aurrecoechea, Cristina, John Brestelli, Brian P. Brunk, Jennifer Dommer, Steve Fischer, Bindu Gajria, Xin Gao, et al. 2009. “PlasmoDB: A Functional Genomic Database for Malaria Parasites.” Nucleic Acids Research 37 (Database issue): D539-43.
Avesson, Lotta, and Guy Barry. 2014. “The Emerging Role of RNA and DNA Editing in Cancer.” Biochimica et Biophysica Acta 1845 (2): 308-16.
Brinton, Margo A., and Mausumi Basu. 2015. “Functions of the 3′ and 5′ Genome RNA Regions of Members of the Genus Flavivirus.” Virus Research 206: 108-19.
Cribari-Neto, Francisco, and Achim Zeileis. 2010. “Beta Regression in R.” Journal of Statistical Software 34 (1): 1-24.
Donald, Claire L., Benjamin Brennan, Stephanie L. Cumberworth, Veronica V. Rezelj, Jordan J. Clark, Marli T. Cordeiro, Rafael Freitas de Oliveira Franca, et al. 2016. “Full Genome Sequence and sfRNA Interferon Antagonist Activity of Zika Virus from Recife, Brazil.” PLoS Neglected Tropical Diseases 10 (10): e0005048.
Drummond, A. J., A. Rambaut, B. Shapiro, and O. G. Pybus. 2005. “Bayesian Coalescent Inference of Past Population Dynamics from Molecular Sequences.” Molecular Biology and Evolution 22 (5): 1185-92.
Drummond, Alexei J., Marc A. Suchard, Dong Xie, and Andrew Rambaut. 2012. “Bayesian Phylogenetics with BEAUti and the BEAST 1.7.” Molecular Biology and Evolution 29 (8): 1969-73.
Faria, Nuno Rodrigues, Raimunda do Socorro da Silva Azevedo, Moritz U. G. Kraemer, Renato Souza, Mariana Sequetin Cunha, Sarah C. Hill, Julien Thézé, et al. 2016. “Zika Virus in the Americas: Early Epidemiological and Genetic Findings.” Science 352 (6283): 345-49.
Faye, Oumar, Ousmane Faye, Diawo Diallo, Mawlouth Diallo, Manfred Weidmann, and Amadou Alpha Sall. 2013. “Quantitative Real-Time PCR Detection of Zika Virus and Evaluation with Field-Caught Mosquitoes.” Virology Journal 10 (October): 311.
Ferreira, Marco A. R., and Marc A. Suchard. 2008. “Bayesian Analysis of Elapsed Times in Continuous-Time Markov Chains.” The Canadian Journal of Statistics=Revue Canadienne de Statistique 36 (3). Wiley-Blackwell: 355-68.
Gire, Stephen K., Augustine Goba, Kristian G. Andersen, Rachel S. G. Sealfon, Daniel J. Park, Lansana Kanneh, Simbirie Jalloh, et al. 2014. “Genomic Surveillance Elucidates Ebola Virus Origin and Transmission during the 2014 Outbreak.” Science 345 (6202): 1369-72.
Hemert, Formijn van, and Ben Berkhout. 2016. “Nucleotide Composition of the Zika Virus RNA Genome and Its Codon Usage.” Virology Journal 13 (June): 95.
Henikoff, S., and J. G. Henikoff. 1992. “Amino Acid Substitution Matrices from Protein Blocks.” Proceedings of the National Academy of Sciences of the United States of America 89 (22): 10915-19.
Josse, Julie, and Francois Husson. 2016. “missMDA: A Package for Handling Missing Values in Multivariate Data Analysis.” Journal of Statistical Software 70 (1): 1-31.
Katoh, Kazutaka, and Daron M. Standley. 2013. “MAFFT Multiple Sequence Alignment Software Version 7: Improvements in Performance and Usability.” Molecular Biology and Evolution 30 (4): 772-80.
Kearse, Matthew, Richard Moir, Amy Wilson, Steven Stones-Havas, Matthew Cheung, Shane Sturrock, Simon Buxton, et al. 2012. “Geneious Basic: An Integrated and Extendable Desktop Software Platform for the Organization and Analysis of Sequence Data.” Bioinformatics 28 (12): 1647-49.
Lanciotti, Robert S., Olga L. Kosoy, Janeen J. Laven, Jason O. Velez, Amy J. Lambert, Alison J. Johnson, Stephanie M. Stanfield, and Mark R Duffy. 2008. “Genetic and Serologic Properties of Zika Virus Associated with an Epidemic, Yap State, Micronesia, 2007.” Emerging Infectious Diseases 14 (8): 1232-39.
Lê, S., J. Josse, and F. Husson. 2008. “FactoMineR: An R Package for Multivariate Analysis.” Journal of Statistical Software. Citeseer. http://citeseerx.ist.psu.edu/viewdoc/download?doi=10.1.1.422.7829&rep-rep 1&type-pdf.
Matranga, Christian B., Kristian G. Andersen, Sarah Winnicki, Michele Busby, Adrianne D. Gladden, Ryan Tewhey, Matthew Stremlau, et al. 2014. “Enhanced Methods for Unbiased Deep Sequencing of Lassa and Ebola RNA Viruses from Clinical and Biological Samples.” Genome Biology 15 (11): 519.
Minh, Bui Quang, Minh Anh Thi Nguyen, and Arndt von Haeseler. 2013. “Ultrafast Approximation for Phylogenetic Bootstrap.” Molecular Biology and Evolution 30 (5): 1188-95.
Morlan, John D., Kunbin Qu, and Dominick V. Sinicropi. 2012. “Selective Depletion of rRNA Enables Whole Transcriptome Profiling of Archival Fixed Tissue.” PloS One 7 (8): e42882.
Nguyen, Lam-Tung, Heiko A. Schmidt, Arndt von Haeseler, and Bui Quang Minh. 2015. “IQ-TREE: A Fast and Effective Stochastic Algorithm for Estimating Maximum-Likelihood Phylogenies.” Molecular Biology and Evolution 32 (1): 268-74.
Park, Daniel J., Gytis Dudas, Shirlee Wohl, Augustine Goba, Shannon L. M. Whitmer, Kristian G. Andersen, Rachel S. Sealfon, et al. 2015. “Ebola Virus Epidemiology, Transmission, and Evolution during Seven Months in Sierra Leone.” Cell 161 (7): 1516-26.
Pyke, Alyssa T., Michelle T. Daly, Jane N. Cameron, Peter R. Moore, Carmel T. Taylor, Glen R. Hewitson, Jan L. Humphreys, and Richard Gair. 2014. “Imported Zika Virus Infection from the Cook Islands into Australia, 2014.” PLoS Currents 6 (June). doi:10.1371/currents.outbreaks.4635a54dbffba2156fb2fd76dc49f65e.
Rambaut, Andrew. 2014. “FigTree. Version 1.4.2.” Edinburgh, UK. Inst. Evol. Biol., Univ. Edinburgh. http://tree.bio.ed.ac.uk/software/figtree/.
Rambaut, Andrew, Tommy T. Lam, Luiz Max Carvalho, and Oliver G. Pybus. 2016. “Exploring the Temporal Structure of Heterochronous Sequences Using TempEst (formerly Path-O-Gen).” Virus Evolution 2 (1): vew007.
R Core Team. 2016. “R: A Language and Environment for Statistical Computing.” R Foundation for Statistical Computing. https://www.R-project.org/.
Shapiro, Beth, Andrew Rambaut, and Alexei J. Drummond. 2006. “Choosing Appropriate Substitution Models for the Phylogenetic Analysis of Protein-Coding Sequences.” Molecular Biology and Evolution 23 (1): 7-9.
Tomkins-Tinch, Chris, Simon Ye, Hayden Metsky, Irwin Jungreis, Rachel Sealfon, Xiao Yang, Kristian Andersen, Mike Lin, and Daniel Park. 2016. Broadinstitute/Viral-Ngs: V1.13.3. Zenodo. doi: 10.5281/zenodo.200428.
Wood, Derrick E., and Steven L. Salzberg. 2014. “Kraken: Ultrafast Metagenomic Sequence Classification Using Exact Alignments.” Genome Biology 15 (3): R46.
Yarza, Pablo, Michael Richter, Jorg Peplies, Jean Euzeby, Rudolf Amann, Karl-Heinz Schleifer, Wolfgang Ludwig, Frank Oliver Glöckner, and Ramon Rosselló-Móra. 2008. “The All-Species Living Tree Project: A 16S rRNA-Based Phylogenetic Tree of All Sequenced Type Strains.” Systematic and Applied Microbiology 31 (4): 241-50.

1. A method for developing probes and primers to pathogens, comprising:
providing a set of input genomic sequences to one or more target pathogens;
generating a set of target sequences from the set of input genomic sequences;
applying a set cover solving process to the set of target sequences to identify one or more target amplification sequences, wherein the one or more target amplification sequences are highly conserved target sequences shared between the set of input genomic sequences of the one or more target pathogens; and
generating one or more primers, one or more probes, or a primer pair and probe combination based on the one or more target amplification sequences.

2. The method of claim 1, wherein the set of input genomic sequences represent genomic sequences from two or more variants of the one or more target pathogens.

3. The method of claim 1, wherein the set of input genomic sequences are obtained from a metagenomic sample.

4. The method of claim 3, wherein the metagenomic sample is obtained from one or more vector species of the one or more target pathogens.

5. The method of claim 4, wherein the one or more vector species are one or more species of mosquito.

6. The method of claim 1, wherein the one or more target pathogens is one or more viral pathogens.

7. The method of claim 6, wherein the one or more viral pathogens is Zika virus, chikungunva virus, or dengue virus.

8. The method of claim 7, wherein the one or more viral pathogens is Zika virus or chikungunya virus.

9. The method of claim 1, wherein the one or more target pathogens is a parasitic pathogen.

10. The method of claim 1, wherein the target sequences are fragmented to a size that is approximately equal to a size of an amplicon for detection using a nucleic acid amplification assay.

11. The method of claim 10, wherein the size of the target sequence is 100 to 500 base pairs.

12. The method of claim 1, wherein each nucleotide of the set of input genomic sequences is considered an element of universe of the set cover solving process and wherein each element is considered covered if the target sequence aligns to some portion of a genomic reference sequence.

13. A method for detecting one or more pathogens comprising:
contacting a sample with one or more primers and/or probes generated using the method of claim 1;
detecting amplification of one or more pathogen target sequences using a nucleic acid amplification method and the one or more primers and/or probes, wherein detection of a target sequence indicates a presence of the one or more pathogens in the sample.

14. The method of claim 13, wherein the nucleic acid amplification method is quantitative PCR and the one or more primers and/or probes comprise forward and reverse primers and a probe modified with a detectable label.

15. The method of claim 14, wherein the forward primer comprises one of SEQ ID NOs: 1, 5, 9, 13, 17, 21, 25, 29, 33, 37, or 41, the reverse primer comprises one of SEQ ID NOs: 2, 6, 10, 14, 18 22, 26, 30, 34, 38, or 42, and the probe comprises one of SEQ ID NOs: 3, 7, 11, 15, 19, 23, 27, 31, 35, 39, or 45.

16. The method of claim 13, wherein the one or more primers and/or probes are configured to detect one or more non-synonymous single nucleotide polymorphisms (SNPs) listed in Tables 3 or 7.

17. A method for detecting Zika virus and/or chikungunya virus in samples, comprising
contacting a sample with a forward and reverse primer and a probe with a detectable label, wherein the forward primer comprises one or more of SEQ ID NOs: 1, 5, 9, 13, 17, 21, 25, 29, 33, 37, or 41, the reverse primer comprises one of more of SEQ ID NOs: 2, 6, 10, 14, 18 22, 26, 30, 34, 38, or 42, and the probe comprises one or more of SEQ ID NOs: 3, 7, 11, 15, 19, 23, 27, 31, 35, 39, or 45;
detecting amplification of one or more target sequences through a quantitative PCR assay using the forward and reverse primers and the probe, wherein detection of the one or more target sequences indicates the presence of Zika virus, chikungunya virus, or both.

18. A kit comprising the primers and/or probes of claim 1.

19. A kit comprising the primers and/or probes of claim 17.


SEQUENCE LISTING (47 sequences)

SEQ ID NO: 1 - DNA, 22 bp, Artificial Sequence
  Synthetic primer
tcctttaacgaggatccattgg

SEQ ID NO: 2 - DNA, 23 bp, Artificial Sequence
  Synthetic primer
cgagctttttaactgcagcaact

SEQ ID NO: 3 - DNA, 20 bp, Zika virus
ggcttgaagcaagaatgctc

SEQ ID NO: 4 - DNA, 20 bp, Zika virus
ccctcaatggctgctacttt

SEQ ID NO: 5 - DNA, 24 bp, Zika virus
agatggcctcatagcctcgctcta

SEQ ID NO: 6 - DNA, 100 bp, Zika virus
ggcttgaagcaagaatgctccttgacaatatttacctccaagatggcctcatagcctcgctctatcgacctgaggccgacaaagtagcagccattgaggg

SEQ ID NO: 7 - DNA, 21 bp, Zika virus
attgaggaatggtgctgtagg

SEQ ID NO: 8 - DNA, 21 bp, Zika virus
gttctttcctgggccttatct

SEQ ID NO: 9 - DNA, 24 bp, Zika virus
aagacggctgctggtatggaatgg

SEQ ID NO: 10 - DNA, 100 bp, Zika virus
attgaggaatggtgctgtagggaatgcacaatgcccccactatcgtttcgagcaaaagacggctgctggtatggaatggagataaggcccaggaaagaac

SEQ ID NO: 11 - DNA, 20 bp, Zika virus
tcatgaagaacccrtgytgg

SEQ ID NO: 12 - DNA, 20 bp, Zika virus
ctcagccgccatrtgraaga

SEQ ID NO: 13 - DNA, 21 bp, Zika virus
tgcaaagctatgggtggaaca

SEQ ID NO: 14 - DNA, 88 bp, Zika virus
tcatgaagaacccgtgttggtgcaaagctatgggtggaacatagtccgtcttaagagtggggtggacgtctttcatatggcggctgag

SEQ ID NO: 15 - DNA, 20 bp, Zika virus
agyygaytgggthccaactg

SEQ ID NO: 16 - DNA, 22 bp, Zika virus
ytcctcaatccacactctrttc

SEQ ID NO: 17 - DNA, 24 bp, Zika virus
acctggtcaatccatggaaaggga

SEQ ID NO: 18 - DNA, 109 bp, Zika virus
agttgactgggttccaactgggagaactacctggtcaatccatggaaagggagaatggatgaccactgaagacatgcttgtggtgtggaacagagtgtggattgaggag

SEQ ID NO: 19 - DNA, 21 bp, Zika virus
ccayttcaacaarctsyayct

SEQ ID NO: 20 - DNA, 19 bp, Zika virus
tttgcwarcargcagtctc

SEQ ID NO: 21 - DNA, 22 bp, Zika virus
tgccgccaccaagatgaactga

SEQ ID NO: 22 - DNA, 135 bp, Zika virus
ccacttcaacaagctccatctcaaggacgggaggtccattgtggttccctgccgccaccaagatgaactgattggccgggcccgcgtctctccaggggcgggatggagcatccgggagactgcttgcctagcaaa

SEQ ID NO: 23 - DNA, 17 bp, Zika virus
tsyagggartgcacaat

SEQ ID NO: 24 - DNA, 24 bp, Zika virus
actaagttrctytctggttcytty

SEQ ID NO: 25 - DNA, 24 bp, Zika virus
tggtatggaatggagataaggccc

SEQ ID NO: 26 - DNA, 102 bp, Zika virus
agggagtgcacaatgcccccactgtcgttccgggctaaagatggctgttggtatggaatggagataaggcccaggaaagaaccagaaagcaacttagtaagg

SEQ ID NO: 27 - DNA, 20 bp, Zika virus
agagaccctgggagagaaat

SEQ ID NO: 28 - DNA, 19 bp, Zika virus
ctcggtgatgcctgacttt

SEQ ID NO: 29 - DNA, 24 bp, Zika virus
agatgtcggccctggagttctact

SEQ ID NO: 30 - DNA, 88 bp, Zika virus
agagaccctgggagagaaatggaaggcccgcttgaaccagatgtcggccctggagttctactcctacaaaaagtcaggcatcaccgag

SEQ ID NO: 31 - DNA, 19 bp, Chikungunya virus
tttgcaagctccagatcca

SEQ ID NO: 32 - DNA, 23 bp, Chikungunya virus
gtagctgtagtgcgtacctattt

SEQ ID NO: 33 - DNA, 20 bp, Chikungunya virus
gagaagctcagaggacccgt

SEQ ID NO: 34 - DNA, 89 bp, Chikungunya virus
tttgcaagctccagatccaacttcgagaagctcagaggacccgtcataactttgtacggcggtcctaaataggtacgcactacagctac

SEQ ID NO: 35 - DNA, 21 bp, Chikungunya virus
cgttctcgcatctagccataa

SEQ ID NO: 36 - DNA, 23 bp, Chikungunya virus
tgatcccgactcaaccatcctgg

SEQ ID NO: 37 - DNA, 22 bp, Chikungunya virus
gtacttcctgtccgacatcatc

SEQ ID NO: 38 - DNA, 110 bp, Chikungunya virus
cgttctcgcatctagccataaaactaatagagcaggaaattgatcccgactcaaccatcctggatataggtagtgcgccagcaaggaggatgatgtcggacaggaagtac

SEQ ID NO: 39 - DNA, 18 bp, Chikungunya virus
cccgactcaaccatcctg

SEQ ID NO: 40 - DNA, 18 bp, Chikungunya virus
gcagacgcagtggtactt

SEQ ID NO: 41 - DNA, 22 bp, Chikungunya virus
ccagcaaggaggatgatgtcgg

SEQ ID NO: 42 - DNA, 90 bp, Chikungunya virus
ccccgactcaaccatcctggatatcggcagtgcgccagcaaggaggatgatgtcggacaggaagtaccaggaagtaccactgcgtctgcc

SEQ ID NO: 43 - DNA, 19 bp, Dengue virus
aaccwacgraaraagrcgv

SEQ ID NO: 44 - DNA, 20 bp, Dengue virus
grgaawctcttygyyarctg

SEQ ID NO: 45 - DNA, 17 bp, Dengue virus
tcaatatgctgaaacgc

SEQ ID NO: 46 - DNA, 95 bp, Dengue virus
aacctacgaaaaaagacggctcgaccgtctttcaatatgctgaaacgcgcgagaaaccgcgtgtcaactgtttcacagttggcgaagagattctc

SEQ ID NO: 47 - DNA, 25 bp, Dengue virus
cgtctttcaatatgctgaaacgcgc